SEQUENCE LISTING (20 sequences)

SEQ ID NO: 1 - DNA, 365 bp, Artificial Sequence
  source: /note=“Description of Artificial Sequence Synthetic polynucleotide”
aggtgcagctggtggagtctgggggaaacttggaacagcctggggggtcccttagactctcctgtacagcctctggattcacctttagcagatctgccatgaactgggtccgccgggctccagggaaggggctggagtgggtctcaggaattagtggtagtggtggtcgaacatactacgcagactccgtgaagggccggttcaccatctccagagacaattccaagaatacgctatatctgcaaatgaacagcctgagcgccgaggacacggccgcatattactgtgcgaaagattcgtatactaccagttggtacggaggtatggacgtctggggccacgggaccacggtcaccgtctcctca

SEQ ID NO: 2 - protein, 121 aa, Artificial Sequence
  source: /note=“Description of Artificial Sequence Synthetic polypeptide”
VQLVESGGNLEQPGGSLRLSCTASGFTFSRSAMNWVRRAPGKGLEWVSGISGSGGRTYYADSVKGRFTISRDNSKNTLYLQMNSLSAEDTAAYYCAKDSYTTSWYGGMDVWGHGTTVTVSS

SEQ ID NO: 3 - DNA, 24 bp, Artificial Sequence
  source: /note=“Description of Artificial Sequence Synthetic oligonucleotide”
ggattcacctttagcagatctgcc

SEQ ID NO: 4 - protein, 8 aa, Artificial Sequence
  source: /note=“Description of Artificial Sequence Synthetic peptide”
GFTFSRSA

SEQ ID NO: 5 - DNA, 24 bp, Artificial Sequence
  source: /note=“Description of Artificial Sequence Synthetic oligonucleotide”
attagtggtagtggtggtcgaaca

SEQ ID NO: 6 - protein, 8 aa, Artificial Sequence
  source: /note=“Description of Artificial Sequence Synthetic peptide”
ISGSGGRT

SEQ ID NO: 7 - DNA, 45 bp, Artificial Sequence
  source: /note=“Description of Artificial Sequence Synthetic oligonucleotide”
gcgaaagattcgtatactaccagttggtacggaggtatggacgtc

SEQ ID NO: 8 - protein, 15 aa, Artificial Sequence
  source: /note=“Description of Artificial Sequence Synthetic peptide”
AKDSYTTSWYGGMDV

SEQ ID NO: 9 - DNA, 323 bp, Artificial Sequence
  source: /note=“Description of Artificial Sequence Synthetic polynucleotide”
acatccagatgacccagtctccatcttccgtgtctgcatctgtaggagacagagtcaccatcacttgtcgggcgagtcagggtattttcagctggttagcctggtatcagcagaaaccaggaaaagcccctaagctcctgatctatgctgcttccagtttacaaagtggggtcccatcaagattcagcggcagtggatctgggacagatttcactctcaccatcagcagcctgcagcctgaggattttgcaatttactattgtcaacaggctaacagtgtcccgatcaccttcggccaagggacacgactggagattaaacga

SEQ ID NO: 10 - protein, 107 aa, Artificial Sequence
  source: /note=“Description of Artificial Sequence Synthetic polypeptide”
IQMTQSPSSVSASVGDRVTITCRASQGIFSWLAWYQQKPGKAPKLLIYAASSLQSGVPSRFSGSGSGTDFTLTISSLQPEDFAIYYCQQANSVPITFGQGTRLEIKR

SEQ ID NO: 11 - DNA, 18 bp, Artificial Sequence
  source: /note=“Description of Artificial Sequence Synthetic oligonucleotide”
cagggtattttcagctgg

SEQ ID NO: 12 - protein, 6 aa, Artificial Sequence
  source: /note=“Description of Artificial Sequence Synthetic peptide”
QGIFSW

SEQ ID NO: 13 - DNA, 9 bp, Artificial Sequence
  source: /note=“Description of Artificial Sequence Synthetic oligonucleotide”
gctgcttcc

SEQ ID NO: 14 - protein, 3 aa, Artificial Sequence
  source: /note=“Description of Artificial Sequence Synthetic peptide”
AAS

SEQ ID NO: 15 - DNA, 27 bp, Artificial Sequence
  source: /note=“Description of Artificial Sequence Synthetic oligonucleotide”
caacaggctaacagtgtcccgatcacc

SEQ ID NO: 16 - protein, 9 aa, Artificial Sequence
  source: /note=“Description of Artificial Sequence Synthetic peptide”
QQANSVPIT

SEQ ID NO: 17 - DNA, 1349 bp, Artificial Sequence
  source: /note=“Description of Artificial Sequence Synthetic polynucleotide”
aggtgcagctggtggagtctgggggaaacttggaacagcctggggggtcccttagactctcctgtacagcctctggattcacctttagcagatctgccatgaactgggtccgccgggctccagggaaggggctggagtgggtctcaggaattagtggtagtggtggtcgaacatactacgcagactccgtgaagggccggttcaccatctccagagacaattccaagaatacgctatatctgcaaatgaacagcctgagcgccgaggacacggccgcatattactgtgcgaaagattcgtatactaccagttggtacggaggtatggacgtctggggccacgggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 18 - protein, 448 aa, Artificial Sequence
  source: /note=“Description of Artificial Sequence Synthetic polypeptide”
VQLVESGGNLEQPGGSLRLSCTASGFTFSRSAMNWVRRAPGKGLEWVSGISGSGGRTYYADSVKGRFTISRDNSKNTLYLQMNSLSAEDTAAYYCAKDSYTTSWYGGMDVWGHGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - DNA, 644 bp, Artificial Sequence
  source: /note=“Description of Artificial Sequence Synthetic polynucleotide”
acatccagatgacccagtctccatcttccgtgtctgcatctgtaggagacagagtcaccatcacttgtcgggcgagtcagggtattttcagctggttagcctggtatcagcagaaaccaggaaaagcccctaagctcctgatctatgctgcttccagtttacaaagtggggtcccatcaagattcagcggcagtggatctgggacagatttcactctcaccatcagcagcctgcagcctgaggattttgcaatttactattgtcaacaggctaacagtgtcccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 20 - protein, 213 aa, Artificial Sequence
  source: /note=“Description of Artificial Sequence Synthetic polypeptide”
IQMTQSPSSVSASVGDRVTITCRASQGIFSWLAWYQQKPGKAPKLLIYAASSLQSGVPSRFSGSGSGTDFTLTISSLQPEDFAIYYCQQANSVPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2021/0230265 A1
(43) Pub. Date: Jul. 29, 2021

(54) METHODS FOR TREATING COPD BY ADMINISTERING AN IL-33 ANTAGONIST

(71) Applicants: Sanofi Biotechnology, Paris (FR); Regeneron Pharmaceuticals, Inc., Tarrytown, NY (US)

(72) Inventors: Raolat Abdulai, Bridgewater, NJ (US); Alexander Boddy, Bridgewater, NJ (US); Deborah Dukovic, Bridgewater, NJ (US); Helene Goulaouic, Paris (FR); Andreas Jessel, Bridgewater, NJ (US); Xiaodong Luo, Bridgewater, NJ (US); Heribert Staudinger, Bridgewater, NJ (US); Ariel Teper, Bridgewater, NJ (US); Marcella Ruddy, Tarrytown, NY (US); Nikhil Amin, Tarrytown, NY (US); Sivan Harel, Tarrytown, NY (US); Chad Nivens, Tarrytown, NY (US)

(21) Appl. No.: 17/112,198

(22) Filed: Dec. 4, 2020

Related U.S. Application Data
(60) Provisional application No. 63/082,502, filed on Sep. 24, 2020.
(60) Provisional application No. 62/964,966, filed on Jan. 23, 2020.
(60) Provisional application No. 62/944,878, filed on Dec. 6, 2019.

(51) Int. Cl. C07K 16/24; A61K 9/00; A61K 39/395; A61P 11/00; A61K 45/06

(52) U.S. Cl. CPC C07K 16/244; A61K 9/0019; A61K 39/3955; A61K 2039/545; A61K 45/06; C07K 2317/565; C07K 2317/76; A61P 11/00

(57) ABSTRACT

Methods for treating or preventing COPD and associated conditions in a patient are provided. Methods comprising administering to a subject in need thereof a therapeutic composition comprising an interleukin-33 (IL-33) antagonist, such as an anti-IL-33 antibody or antigen-binding fragment thereof, are provided.

### RELATED APPLICATIONS

This application claims priority to U.S. Provisional Patent Application Serial Nos. 62/944,878, filed Dec. 6, 2019, 62/964,966, filed Jan. 23, 2020, and 63/082,502, filed Sep. 24, 2020. The entire disclosure of each of these applications is hereby incorporated herein by reference in its entirety.

### FIELD OF THE INVENTION

The invention relates to the treatment and/or prevention of chronic obstructive pulmonary disease (COPD), and related conditions. More specifically, the invention relates to the administration of an interleukin-33 (IL-33) antagonist to treat or prevent COPD and/or decrease acute exacerbation of COPD (AECOPD) events in a patient in need thereof.

### BACKGROUND

Chronic obstructive pulmonary disease (COPD) is a heterogeneous syndrome associated with an abnormal inflammatory immune response of the lung to noxious particles and gases. Chronic inflammation causes structural changes, narrowing of the small airways, and destruction of the lung parenchyma that leads to the loss of alveolar attachments to the small airways and decreases lung elastic recoil. It results in progressive airflow obstruction that is only partly reversible or even irreversible. The inflammation component of COPD is thought to involve many cell types including structural cells, T lymphocytes, neutrophils, macrophages, and their biological products. In some patients, there may also be an increase of eosinophils, T-helper (Th) 2 or Group 2 innate lymphoid cells, especially where there is clinical overlap with asthma. The main cause of COPD is smoking tobacco, but other factors have been identified, such as air pollution, occupational exposure, and genetic susceptibility. The most common respiratory symptoms include chronic dyspnea, cough and/or sputum production. The disease is further aggravated by exacerbations, particularly for severe COPD. These are most often due to viral and bacterial infections of the lungs which trigger the inflammatory response, tissue destruction, and the resultant hypoxia. Exacerbations in COPD patients are associated with rapid disease progression (rate of lung function decline over time) and increased risk of mortality. Medical comorbidities such as cardiovascular disease, diabetes, lung cancer, skeletal muscle dysfunction, osteoporosis, psychological disturbances, and metabolic syndrome are common among COPD patients and occur across the spectrum of disease severity.

Chronic obstructive pulmonary disease is a highly prevalent, serious and progressive disease resulting in significant morbidity, mortality, and economic burden (Adeloye et al. Global and regional estimates of COPD prevalence: systematic review and meta-analysis. J Glob Health. 2015 December; 5(2):020415; Guarascio et al. The clinical and economic burden of chronic obstructive pulmonary disease in the USA. Clinicoecon. Outcomes Res. 2013 Jun. 17; 5:235-45). In the US alone, there are more than 12 million diagnosed patients, and the incidence of COPD is expected to grow rapidly with an aging population. COPD is a progressive and partly reversible or irreversible inflammatory lung disease that is periodically punctuated by disease exacerbations that result in long term disability and mortality. Globally, there are around 3 million deaths attributed to COPD annually. With the increasing prevalence of smoking in developing countries, and aging populations in high-income countries, the prevalence is expected to rise and the number of deaths to reach 4.5 million by 2030.

The standard of care for moderate COPD starts with bronchodilators (such as long-acting muscarinic antagonists (LAMA) or long-acting β2 agonists (LABA)), and as disease progresses, bronchodilators are combined with other drugs such as inhaled corticosteroids (ICS), and phosphodiesterase type 4 (PDE-4) inhibitors (roflumilast) (Aaron et al. Tiotropium in Combination with Placebo, Salmeterol, or Fluticasone-Salmeterol for Treatment of Chronic Obstructive Pulmonary Disease: a randomized trial. Ann Intern Med. 2007 Apr. 17; 146(8):545-55; Calverley et al. Roflumilast in symptomatic chronic obstructive pulmonary disease: two randomized clinical trials. Lancet. 2009 Aug. 29; 374(9691):685-94). The major limitations of the existing agents for COPD include modest efficacy and risk of respiratory infections. Oral or systemic corticosteroids are reserved for treatment of exacerbations given their unacceptable long-term safety profile in the COPD population. No approved therapeutic agent blocks the decline in forced expiratory volume in 1 second (FEV1) over time or modifies the progressive disease course of COPD.

Thus, significant unmet medical needs continue to exist in the growing population of patients with COPD. Accordingly, a need exists in the art for novel targeted therapies for the treatment and/or prevention of COPD and/or the reduction of acute exacerbation of COPD (AECOPD) events.

### BRIEF SUMMARY OF THE INVENTION

In one aspect, a method for treating chronic obstructive pulmonary disease (COPD) in a subject in need thereof comprising administering to the subject an antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16, is provided. In one aspect, an antibody or antigen-binding fragment thereof that specifically binds interleukin-33 (IL-33) and comprises three heavy chain complementary determining region (HCDR) sequences comprising SEQ ID NOs: 4, 6 and 8, and three light chain complementary determining region (LCDR) sequences comprising SEQ ID NOs: 12, 14 and 16 is provided for use to treat treating chronic obstructive pulmonary disease (COPD) in a subject in need thereof.

In certain exemplary embodiments, one or more COPD-associated parameter(s) are improved in the subject. In certain exemplary embodiments, the one or more COPD-associated parameter(s) are selected from the group consisting of annualized rate of moderate-to-severe acute exacerbations of COPD (AECOPD), annualized rate of severe acute exacerbations of COPD (AECOPD), forced expiratory volume in 1 second (FEV1), peak expiratory flow (PEF), forced vital capacity (FVC), forced expiratory flow (FEF) 25%-75%, fractional exhaled nitric oxide (FeNO), frequency or dosage of a chronic obstructive pulmonary disease (COPD) reliever medication, frequency or dosage of a systemic corticosteroid, frequency or dosage of an antibiotic, daily steps, frequency or dosage of an oral corticosteroid, resting oxygen saturation, and resting respiratory rate. In certain exemplary embodiments, pre-bronchodilator FEV1 is improved in the subject. In certain exemplary embodiments, annualized rate of AECOPD is reduced in the subject.

In certain exemplary embodiments, a score is improved in the subject on one or more questionnaires or assessments selected from the group consisting of COPD Assessment Test (CAT), St. George's Respiratory Questionnaire (SGRQ), Exacerbations of Chronic Obstructive Pulmonary Disease Tool (EXACT), Evaluating Respiratory Symptoms in COPD (E-RS), Body mass index, airflow Obstruction, Dyspnea, Exercise performance (BODE) Index, and Euro Quality of Life-5 Dimension questionnaire (EQ-5D).

In certain exemplary embodiments, the COPD is moderate-to-severe COPD that is not well-controlled on a background therapy. In certain exemplary embodiments, the background therapy comprises therapy with at least two of the following: a long-acting β2 adrenergic agonist (LABA), a long-acting muscarinic antagonist (LAMA), and an inhaled corticosteroid (ICS). In certain exemplary embodiments, the background therapy comprises a LABA and a LAMA. In certain exemplary embodiments, the background therapy comprises a LABA and an ICS. In certain exemplary embodiments, the background therapy comprises a LAMA and an ICS. In certain exemplary embodiments, the background therapy comprises therapy with a LABA, a LAMA, and an ICS.

In certain exemplary embodiments, the antibody or antigen-binding fragment thereof comprises a heavy chain variable region (HCVR) comprising the amino acid sequence of SEQ ID NO: 2 and a light chain variable region (LCVR) comprising the amino acid sequence of SEQ ID NO: 10. In certain exemplary embodiments, the antibody comprises a heavy chain comprising the amino acid sequence of SEQ ID NO; 18, and a light chain comprising the amino acid sequence of SEQ ID NO: 20 (also known as SAR440340, REGN3500, or itepekimab).

In certain exemplary embodiments, the subject has a blood eosinophil count of greater than or equal to about 250 cells per μl or less than 250 cells per μL prior to treatment. In certain exemplary embodiments, the subject has a blood eosinophil count of greater than or equal to about 250 cells per μl prior to treatment. In certain exemplary embodiments, the subject has a blood eosinophil count of greater than or equal to about 300 cells per μl or less than 300 cells per μL prior to treatment. In certain exemplary embodiments, the subject has a blood eosinophil count of greater than or equal to about 300 cells per μl prior to treatment. In certain exemplary embodiments, pre-bronchodilator FEV1 is improved. In certain exemplary embodiments, post-bronchodilator FEV1 is improved. In certain exemplary embodiments, pre-bronchodilator FVC is improved.

In certain exemplary embodiments, the subject is a current smoker, former smoker or a non-smoker. In certain exemplary embodiments, the subject is a former smoker. In certain exemplary embodiments, the former smoker has a history of smoking greater than or equal to 10 packs per year. In certain exemplary embodiments, the former smoker has quit smoking for at least 6 months. In certain exemplary embodiments, the smoker intends to permanently quit smoking

In certain exemplary embodiments, annualized rate of moderate-to-severe AECOPD events is reduced in the subject. In certain exemplary embodiments, time to first moderate-to-severe AECOPD event is reduced. In certain exemplary embodiments, pre-bronchodilator FEV1 is improved. In certain exemplary embodiments, post-bronchodilator FEV1 is improved. In certain exemplary embodiments, pre-bronchodilator FVC is improved. In certain exemplary embodiments, the level of blood eosinophils is reduced.

In certain exemplary embodiments, annualized rate of severe AECOPD events is reduced in the subject. In certain exemplary embodiments, time to first severe AECOPD event is reduced. In certain exemplary embodiments, pre-bronchodilator FEV1 is improved. In certain exemplary embodiments, post-bronchodilator FEV1 is improved. In certain exemplary embodiments, rate of pre-bronchodilator FEV1 decline is decreased. In certain exemplary embodiments, rate of post-bronchodilator FEV1 decline is decreased. In certain exemplary embodiments, pre-bronchodilator FVC is improved. In certain exemplary embodiments, lung function is maintained or lung function decline is reduced. In certain exemplary embodiments, the level of blood eosinophils is reduced. In certain exemplary embodiments, the subject has a high eosinophil blood level and/or is a former smoker.

In certain exemplary embodiments, the antibody or antigen binding fragment thereof is administered at a dose of about 0.1 mg to about 600 mg, about 100 mg to about 400 mg, or about 300 mg. In certain exemplary embodiments, the antibody or antigen binding fragment thereof is administered at a dose of about 300 mg.

In certain exemplary embodiments, the antibody or antigen-binding fragment thereof is administered every week (q1w), every other week (q2w), every three weeks (q3w), every four weeks (q4w), every five weeks (q5w), every 6 weeks (q6w), every seven weeks (q7w), or every eight weeks (q8w). In certain exemplary embodiments, the antibody or antigen-binding fragment thereof is administered every other week (q2w). In certain exemplary embodiments, the antibody or antigen-binding fragment thereof is administered every four weeks (q4w).

In certain exemplary embodiments, pre-bronchodilator FEV1 is improved within 4 weeks of the first administration of the antibody or antigen-binding fragment thereof. In certain exemplary embodiments, pre-bronchodilator FEV1 is maintained during treatment.

In certain exemplary embodiments, the antibody or antigen-binding fragment thereof is administered subcutaneously. In certain exemplary embodiments, the antibody or antigen-binding fragment thereof is administered as two injections. In certain exemplary embodiments, the antibody or antigen-binding fragment thereof is administered subcutaneously using an autoinjector, a needle and syringe, or a pen delivery device.

In another aspect, a method for treating chronic obstructive pulmonary disease (COPD) in a subject in need thereof comprising administering to the subject an initial dose of about 300 mg of an antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16, and one or more subsequent doses of about 300 mg of the antibody or antigen-binding fragment thereof, is provided.

In certain exemplary embodiments, the antibody or antigen-binding fragment thereof comprises a heavy chain variable region (HCVR) comprising the amino acid sequence of SEQ ID NO: 2 and a light chain variable region (LCVR) comprising the amino acid sequence of SEQ ID NO: 10.

In another aspect, a method for treating moderate-to-severe chronic obstructive pulmonary disease (COPD) in a subject in need thereof comprising administering to the subject an initial dose of about 300 mg of an antibody that specifically binds interleukin-33 (IL-33), wherein the antibody comprises a heavy chain comprising the amino acid sequence of SEQ ID NO; 18, and a light chain comprising the amino acid sequence of SEQ ID NO: 20, and one or more subsequent doses of about 300 mg of the antibody, wherein the antibody is administered subcutaneously every other week, is provided. In another aspect, an antibody that specifically binds interleukin-33 (IL-33), wherein the antibody comprises a heavy chain comprising the amino acid sequence of SEQ ID NO; 18, and a light chain comprising the amino acid sequence of SEQ ID NO: 20 is provided for use to treat moderate-to-severe chronic obstructive pulmonary disease (COPD) in a subject in need thereof, wherein the antibody is administered to the subject at an initial dose of about 300 mg and then administered at one or more subsequent doses of about 300 mg, and wherein the antibody is administered subcutaneously every other week.

In another aspect, a method for treating moderate-to-severe chronic obstructive pulmonary disease (COPD) in a subject in need thereof comprising administering to the subject an initial dose of about 300 mg of an antibody that specifically binds interleukin-33 (IL-33), wherein the antibody comprises a heavy chain comprising the amino acid sequence of SEQ ID NO; 18, and a light chain comprising the amino acid sequence of SEQ ID NO: 20, and one or more subsequent doses of about 300 mg of the antibody, wherein the antibody is administered subcutaneously every four weeks, is provided. In another aspect, an antibody that specifically binds interleukin-33 (IL-33), wherein the antibody comprises a heavy chain comprising the amino acid sequence of SEQ ID NO; 18, and a light chain comprising the amino acid sequence of SEQ ID NO: 20 is provided for use to treat moderate-to-severe chronic obstructive pulmonary disease (COPD) in a subject in need thereof, wherein the antibody is administered to the subject at an initial dose of about 300 mg and then administered at one or more subsequent doses of about 300 mg, and wherein the antibody is administered subcutaneously every four weeks.

In certain exemplary embodiments, one or more COPD-associated parameter(s) are improved in the subject.

In certain exemplary embodiments, the one or more chronic obstructive pulmonary disease (COPD)-associated parameter(s) are selected from the group consisting of annualized rate of moderate-to-severe acute exacerbations of COPD (AECOPD), forced expiratory volume in 1 second (FEV1), rate of decline in FEV1, peak expiratory flow (PEF), forced vital capacity (FVC), forced expiratory flow (FEF) 25%-75%, fractional exhaled nitric oxide (FeNO), frequency or dosage of a COPD reliever medication, frequency or dosage of a systemic corticosteroid, and frequency or dosage of an antibiotic.

In certain exemplary embodiments, pre-bronchodilator FEV1 is improved. In certain exemplary embodiments, the annualized rate of moderate-to-severe acute exacerbations of COPD (AECOPD) is reduced in the subject. In certain exemplary embodiments, the annualized rate of severe acute exacerbations of AECOPD is reduced in the subject.

In certain exemplary embodiments, at least two additional therapeutic agents are administered to the subject. In certain exemplary embodiments, the at least two additional therapeutic agents are selected from the group consisting of a long-acting β2 adrenergic agonist (LABA), a long-acting muscarinic antagonist (LAMA), and an inhaled corticosteroid (ICS).

In certain exemplary embodiments, the at least two additional therapeutic agents comprise a LABA and an ICS. In certain exemplary embodiments, the at least two additional therapeutic agents comprise a LAMA and an ICS. In certain exemplary embodiments, a total of three additional therapeutic agents are administered to the subject, including a LABA, a LAMA, and an ICS.

In another aspect, a method for reducing annualized rate of moderate-to-severe acute exacerbations of chronic obstructive pulmonary disease (AECOPD) in a subject having moderate-to-severe chronic obstructive pulmonary disease (COPD) comprising administering to the subject an initial dose of about 300 mg of an antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16, and one or more subsequent doses of about 300 mg of the antibody or antigen-binding fragment thereof, is provided. In another aspect, an antibody or antigen-binding fragment thereof that specifically binds interleukin-33 (IL-33) and comprises three heavy chain complementary determining region (HCDR) sequences comprising SEQ ID NOs: 4, 6 and 8, and three light chain complementary determining region (LCDR) sequences comprising SEQ ID NOs: 12, 14 and 16 is provided for use to reduce annualized rate of moderate-to-severe acute exacerbations of chronic obstructive pulmonary disease (AECOPD) in a subject having moderate-to-severe chronic obstructive pulmonary disease (COPD), wherein said antibody or antigen-binding fragment thereof is administered to the subject at an initial dose of about 300 mg and then administered at one or more subsequent doses of about 300 mg.

In certain exemplary embodiments, the antibody or antigen-binding fragment thereof comprises a heavy chain variable region (HCVR) comprising the amino acid sequence of SEQ ID NO: 2 and a light chain variable region (LCVR) comprising the amino acid sequence of SEQ ID NO: 10.

In another aspect, a method for reducing annualized rate of moderate-to-severe acute exacerbations of chronic obstructive pulmonary disease (AECOPD) in a subject having moderate-to-severe chronic obstructive pulmonary disease (COPD) comprising administering to the subject an initial dose of about 300 mg of an antibody that specifically binds interleukin-33 (IL-33), wherein the antibody comprises a heavy chain comprising the amino acid sequence of SEQ ID NO; 18, and a light chain comprising the amino acid sequence of SEQ ID NO: 20, and one or more subsequent doses of about 300 mg of the antibody, wherein the antibody is administered subcutaneously every other week, is provided. In another aspect, an antibody that specifically binds interleukin-33 (IL-33), wherein the antibody comprises a heavy chain comprising the amino acid sequence of SEQ ID NO; 18, and a light chain comprising the amino acid sequence of SEQ ID NO: 20 is provided for use to reduce annualized rate of moderate-to-severe acute exacerbations of chronic obstructive pulmonary disease (AECOPD) in a subject having moderate-to-severe chronic obstructive pulmonary disease (COPD), wherein the antibody is administered to the subject at an initial dose of about 300 mg and then administered at one or more subsequent doses of about 300 mg, and wherein the antibody is administered subcutaneously every other week.

In another aspect, a method for reducing annualized rate of moderate-to-severe acute exacerbations of chronic obstructive pulmonary disease (AECOPD) in a subject having moderate-to-severe chronic obstructive pulmonary disease (COPD) comprising administering to the subject an initial dose of about 300 mg of an antibody that specifically binds interleukin-33 (IL-33), wherein the antibody comprises a heavy chain comprising the amino acid sequence of SEQ ID NO; 18, and a light chain comprising the amino acid sequence of SEQ ID NO: 20, and one or more subsequent doses of about 300 mg of the antibody, wherein the antibody is administered subcutaneously every other week, wherein the subject is a former smoker, is provided. In another aspect, an antibody that specifically binds interleukin-33 (IL-33), wherein the antibody comprises a heavy chain comprising the amino acid sequence of SEQ ID NO; 18, and a light chain comprising the amino acid sequence of SEQ ID NO: 20 is provided for use to reduce annualized rate of moderate-to-severe acute exacerbations of chronic obstructive pulmonary disease (AECOPD) in a subject being a former smoker and having moderate-to-severe chronic obstructive pulmonary disease (COPD), wherein the antibody is administered to the subject at an initial dose of about 300 mg and then administered at one or more subsequent doses of about 300 mg, and wherein the antibody is administered subcutaneously every other week.

In another aspect, a method for reducing annualized rate of moderate-to-severe acute exacerbations of chronic obstructive pulmonary disease (AECOPD) in a subject having moderate-to-severe chronic obstructive pulmonary disease (COPD) comprising administering to the subject an initial dose of about 300 mg of an antibody that specifically binds interleukin-33 (IL-33), wherein the antibody comprises a heavy chain comprising the amino acid sequence of SEQ ID NO; 18, and a light chain comprising the amino acid sequence of SEQ ID NO: 20, and one or more subsequent doses of about 300 mg of the antibody, wherein the antibody is administered subcutaneously every four weeks, is provided. In another aspect, an antibody that specifically binds interleukin-33 (IL-33), wherein the antibody comprises a heavy chain comprising the amino acid sequence of SEQ ID NO; 18, and a light chain comprising the amino acid sequence of SEQ ID NO: 20 is provided for use to reduce annualized rate of moderate-to-severe acute exacerbations of chronic obstructive pulmonary disease (AECOPD) in a subject having moderate-to-severe chronic obstructive pulmonary disease (COPD), wherein the antibody is administered to the subject at an initial dose of about 300 mg and then administered at one or more subsequent doses of about 300 mg, and wherein the antibody is administered subcutaneously every four weeks.

In another aspect, a method for reducing annualized rate of moderate-to-severe acute exacerbations of chronic obstructive pulmonary disease (AECOPD) in a subject having moderate-to-severe chronic obstructive pulmonary disease (COPD) comprising administering to the subject an initial dose of about 300 mg of an antibody that specifically binds interleukin-33 (IL-33), wherein the antibody comprises a heavy chain comprising the amino acid sequence of SEQ ID NO; 18, and a light chain comprising the amino acid sequence of SEQ ID NO: 20, and one or more subsequent doses of about 300 mg of the antibody, wherein the antibody is administered subcutaneously every four weeks, wherein the subject is a former smoker, is provided. In another aspect, an antibody that specifically binds interleukin-33 (IL-33), wherein the antibody comprises a heavy chain comprising the amino acid sequence of SEQ ID NO; 18, and a light chain comprising the amino acid sequence of SEQ ID NO: 20 is provided for use to reduce annualized rate of moderate-to-severe acute exacerbations of chronic obstructive pulmonary disease (AECOPD) in a subject being a former smoker and having moderate-to-severe chronic obstructive pulmonary disease (COPD), wherein the antibody is administered to the subject at an initial dose of about 300 mg and then administered at one or more subsequent doses of about 300 mg, and wherein the antibody is administered subcutaneously every four weeks.

The foregoing and other features and advantages of the present invention will be more fully understood from the following detailed description of illustrative embodiments taken in conjunction with the accompanying drawings.

### BRIEF DESCRIPTION OF THE FIGURES

FIG. 1 graphically depicts the clinical study described herein at Example 1, showing patient disposition, randomization, and outcome of a study that is designed to assess the efficacy, safety and tolerability of SAR440340, in patients with moderate-to-severe chronic obstructive pulmonary disease (COPD). Stars indicate treatment timepoints that consisted of two injections of 1.5 mL each of SAR440340 or placebo. The variable treatment period was determined by either completion of a 52 week treatment duration or the end of treatment of the last patient completing the planned treatment (EOT visit), whichever occurred earlier. All were to receive at least 24 weeks of treatment. The end of treatment (EOT) visit was to occur 2 weeks after the last administration of the investigational medical product (IMP). The end of study (EOS) visit was to occur 20 weeks after the last administration of IMP.

FIG. 2A-FIG. 2C depict baseline disease characteristics relating to exacerbation history. FIG. 2A shows the number of moderate or severe acute exacerbations of COPD (AECOPD) in the past one year, core data. FIG. 2B shows the number of moderate AECOPD exacerbations in the past one year, core data. FIG. 2C shows the number of severe AECOPD exacerbations in the past one year, core data.

FIG. 3A-FIG. 3E depict baseline disease characteristics relating to smoking. FIG. 3A shows smoking history in placebo and SAR440340 groups. FIG. 3B shows smoking status in placebo and SAR440340 groups in subpopulations with high blood eosinophil levels (EOS ≥250/mm3). FIG. 3D shows smoking status in placebo and SAR440340 groups in subpopulations with low blood eosinophil levels (EOS <250/mm³). FIG. 3C shows total pack per year in placebo and SAR440340 groups. FIG. 3E shows years since smoking cessation in former smokers in placebo and SAR440340 groups.

FIG. 4A-FIG. 4C depict baseline disease characteristics relating to background medications, showing that most patients were on an inhaled corticosteroid (ICS)-containing regimen. FIG. 4A shows a summary of background medications in placebo and SAR440340 groups. FIG. 4B shows the number of participants enrolled in an ICS-containing regimen in the placebo and SAR440340 groups. FIG. 4C shows inhaled corticosteroid dosage in those participants enrolled in an ICS-containing regimen.

FIG. 5A-FIG. 5C depict baseline disease characteristics relating to blood eosinophil levels. FIG. 5A shows blood eosinophil levels at screening. FIG. 5B shows baseline blood eosinophil levels. FIG. 5C shows mean baseline eosinophil count in participants in the placebo group, participants the SAR40340 treatment group, and total participants. FIG. 5C also shows the baseline mean blood eosinophil count for placebo and SAR440340 and percent of participants with high or low baseline eosinophil levels at visit 2, compared to their respective eosinophil count on screening at visit 1.

FIG. 6 shows the annualized rate of moderate-to-severe AECOPD exacerbations in placebo and SAR440340 treatment groups. SAR440340 treatment resulted in an about 18% reduction of AECOPD exacerbations in the combined group that included participants with both high and low eosinophil levels.

FIG. 7A-FIG. 7B depict annualized rate of moderate-to-severe AECOPD exacerbations. FIG. 7A shows adjusted annualized moderate-to-severe AECOPD exacerbations in participants with a low blood eosinophil count, EOS <250. FIG. 7B shows adjusted annualized moderate-to-severe AECOPD exacerbation rate in participants with a high blood eosinophil count, EOS ≥250. SAR440340 treatment resulted in similar reduction in AECOPD exacerbations regardless of baseline EOS count (low: 15% vs. high: 20%).

FIG. 8 depicts statistical analysis of time to first moderate-to-severe AECOPD exacerbation in a combined group of high and low eosinophil subjects either treated with SAR440340 or treated with placebo. A relative reduction of 17% in time to first moderate-to-severe AECOPD event was observed.

FIG. 9A-FIG. 9B depict statistical analysis of time taken to first moderate-to-severe AECOPD. FIG. 9A shows time taken to first moderate-to-severe AECOPD exacerbation in the low eosinophil subgroup, EOS <250. FIG. 9B shows time taken to first moderate-to-severe AECOPD exacerbation in the high eosinophil subgroup, EOS ≥250.

FIG. 10 depicts pre-BD FEV1 least mean squares change from baseline to week 16-24 in the placebo group and the SAR40340 treatment group (high and low EOS). SAR440340 improved pre-bronchodilator (pre-BD) forced expiratory volume in 1 second (FEV1) by 60 mL.

FIG. 11 graphically depicts pre-BD FEV1 mean change from baseline to week 48. SAR440340 had a rapid and sustained effect on pre-BD FEV1.

FIG. 12A-FIG. 12B depict pre-BD FEV1 change from baseline to week 16-24 in high and low eosinophil level subgroups. FIG. 12A shows pre-BD FEV1 change from baseline to week 16-24 versus placebo, in the low eosinophil group, EOS <250. FIG. 12B shows pre-BD FEV1 change from baseline to week 16-24 versus placebo, in the high eosinophil group, EOS ≥250. SAR440340 improved pre-BD FEV1 by 110 mL in the high EOS subgroup.

FIG. 13A-FIG. 13B graphically depict pre-BD FEV1 mean change from baseline to week 44 the high eosinophil level group (FIG. 13B) and to week 48 for the low eosinophil level group (FIG. 13A). SAR440340 treatment led to a rapid and sustained improvement in lung function in the high EOS subgroup.

FIG. 14A-FIG. 14B depict post-BD FEV1 change from baseline to week 24 in a combined group of high and low eosinophil subjects either treated with SAR440340 or treated with placebo. FIG. 14A shows post-BD FEV1, mean vs placebo at week 24. FIG. 14B shows mean change on post-BD from baseline to week 52, versus placebo. There was a modest effect on post-BD FEV1 in the SAR440340 group.

FIG. 15A-FIG. 15B depict post-BD FEV1 change from baseline to week 24 in high and low eosinophil subgroups. FIG. 15A shows post-BD FEV1, week 24, in the low eosinophil group, EOS <250. FIG. 15B shows post-BD FEV1, week 24, in the high eosinophil group, EOS ≥250. There was a 70 mL improvement in post-BD FEV1 in the high EOS subgroup.

FIG. 16A-FIG. 16B depict FEV1 mean change from baseline to week 24. FIG. 16A shows FEV1 mean change from baseline to week 24, versus placebo in the low eosinophil group, EOS <250. FIG. 16B shows FEV1 mean change from baseline to week 24, versus placebo in the high eosinophil group, EOS ≥250. SAR440340 showed a trend towards early and sustained improvement of post-BD FEV1 in the high EOS group.

FIG. 17A-FIG. 17B depict cumulative and annualized rate of moderate-to-severe AECOPD exacerbations in a combined group of high and low eosinophil subjects either treated with SAR440340 or treated with placebo. Data is presented for both current (FIG. 17B) and former smokers (FIG. 17A) as subgroups. SAR440340 treatment led to a 42% reduction in adjusted annualized AECOPD in former smokers.

FIG. 18A-FIG. 18B show pre-BD FEV1 change from baseline in a combined group of high and low eosinophil subjects either treated with SAR440340 or treated with placebo. Data is presented for both current (FIG. 18B) and former smokers (FIG. 18A) as subgroups. SAR440340 led to a 90 mL improvement in pre-BD FEV1.

FIG. 19A-FIG. 19B depict post-BD FEV1 change from baseline in a combined group of high and low eosinophil subjects either treated with SAR440340 or treated with placebo. Data is presented for both current (FIG. 19B) and former (FIG. 19A) smokers as subgroups. SAR440340 led to an improvement in post-BD FEV1 in former smokers.

FIG. 20 depicts the efficacy outcome relationship to smoking status and eosinophil level. The greatest efficacy in preventing AECOPD was observed in former smokers treated with SAR440340 regardless of EOS level.

FIG. 21 shows St. George's Respiratory Questionnaire (SGRQ) score change from baseline in a combined group of participants with both high and low eosinophil levels, showing that there was no change in SGRQ with SAR440340 treatment.

FIG. 22A-FIG. 22B depict SGRQ change from baseline in high and low eosinophil subgroups, showing SAR440340 led to an improvement in SGRQ in the high eosinophil subgroup.

FIG. 22A shows SGRQ change from baseline to week 52, in the low eosinophil group, EOS <250.

FIG. 22B shows SGRQ change from baseline to week 36, in the high eosinophil group, EOS ≥250.

FIG. 23A-FIG. 23D show the change in blood eosinophils from baseline to week 24.

FIG. 23A shows mean change in blood eosinophils from baseline to week 52. FIG. 23B shows median percent change in blood eosinophils from baseline to week 52. FIG. 23C shows percent change from baseline at week 24. These data show that SAR440340 treatment led to a rapid and sustained reduction in blood eosinophils, with an about −42% median change. FIG. 26D shows absolute change from baseline (mean change of −10⁷/mm³) at week 24.

FIG. 24A-FIG. 24B depict mean and median percent change in IgE, showing there was a slight reduction in IgE levels from baseline in the SAR440340 group. FIG. 24A shows mean change in IgE. FIG. 24B shows median percent change in IgE.

FIG. 25A-FIG. 25B depict mean change from baseline in select biomarkers. These data show significant impact of SAR440340 treatment IL-33, but not for sST2. FIG. 25A shows mean change in total IL-33. FIG. 25B shows mean change in sST2.

FIG. 26A-FIG. 26B depict the annualized rates of moderate-to-severe AECOPD events in subgroups of current smokers vs. former smokers in an intent-to-treat population. FIG. 26A shows unadjusted and adjusted annualized moderate-to-severe AECOPD events in former smokers. FIG. 26B shows adjusted and unadjusted annualized moderate-to-severe AECOPD events in current smokers. SAR440340 treatment led to a 42% reduction in AECOPD events in former smokers.

FIG. 27A-FIG. 27B depict the annualized rate of moderate-to-severe AECOPD events in moderate COPD vs severe COPD categories in an intent-to-treat (ITT) population, showing there was no significant difference based on COPD categorization with treatment. FIG. 27A shows adjusted and unadjusted annualized moderate-to-severe AECOPD events, moderate COPD.

FIG. 27B shows adjusted and unadjusted annualized moderate-to-severe AECOPD events, severe COPD.

FIG. 28A-FIG. 28B show pre-BD FEV1 LS mean change from baseline, LS mean, showing that SAR440340 improved pre-BD FEV1 by 60 mL. FIG. 28A shows pre-BD FEV1, LS mean vs PBO, week 16-24. FIG. 28B shows pre-BD FEV1, LS mean vs PBO, week 24.

FIG. 29A-FIG. 29B depict pre-BD FEV1 change from baseline to week 16-24, LS mean, in blood EOS <250 and >250 in an ITT population, showing SAR440340 improved pre-BD FEV1 by 110 mL in the high EOS subgroup. FIG. 295A shows pre-BD FEV1, LS mean vs PBO, week 16-24 in blood EOS <250. FIG. 29B shows pre-BD FEV1, LS mean vs PBO, week 16-24 in blood EOS ≥250.

FIG. 30A-FIG. 30B shows pre-BD FEV1 LS mean change from baseline, LS mean, current smokers vs former smokers in an ITT population, showing SAR440340 led to a 90 mL improvement in pre-BD FEV1. FIG. 30A shows pre-BD FEV1, LS mean vs PBO, week 16-24 in former smokers. FIG. 30B shows pre-BD FEV1, LS mean vs PBO, week 16-24 in current smokers.

FIG. 31A-FIG. 31B show pre-BD FEV1 LS mean change from baseline, LS mean, moderate COPD vs. severe COPD categories in an ITT population, showing SAR440340 led to an improvement in pre-BD FEV1 in patients with lower lung function. FIG. 31A shows pre-BD FEV1, LS mean vs PBO, week 16-24, moderate COPD. FIG. 31B shows pre-BD FEV1, LS mean vs PBO, week 16-24, severe COPD.

FIG. 32A-FIG. 32B show post-BD FEV1 change from baseline to week 24, LS mean in an ITT population, showing that there was a modest effect on post-BD FEV1 in the SAR440340 group. FIG. 32A shows post-BD FEV1, LS mean vs placebo, at week 24. FIG. 32B shows LS mean change from baseline to week 52 versus placebo.

FIG. 33A-FIG. 33B show post-BD FEV1 change from baseline to week 24, LS mean, EOS <250 and EOS >250 in an ITT population, showing that there was a 70 mL improvement in post-BD FEV1 in the high EOS subgroup. FIG. 33A shows post-BD FEV1, LS mean vs PBO, week 24 in EOS <250. FIG. 33B shows post-BD FEV1, LS mean vs PBO, week 24, in EOS ≥250.

FIG. 34A-FIG. 34B depict post-BD FEV1 LS mean change from baseline, LS Mean, current smokers vs former smokers in an ITT population, showing that SAR440340 led to an improvement in post-BD FEV1 in former smokers. FIG. 34A shows post-BD FEV1, LS mean vs PBO, week 24, former smoker. FIG. 34B shows post-BD FEV1, LS mean vs PBO, week 24, current smoker.

FIG. 35A-FIG. 35B depict post-BD FEV1 LS mean change from baseline, LS mean, moderate COPD vs severe COPD categories in an ITT population, showing that SAR440340 led to an improvement in post-BD FEV1 in patients with lower lung function. FIG. 35A shows Post-BD FEV1, LS mean vs PBO, week 24, moderate COPD. FIG. 35B shows post-BD FEV1, LS mean vs PBO, week 24, severe COPD.

FIG. 36A-FIG. 36B depict mean change from baseline in pre-BD and post-BD FeNO, showing there was a reduction in FeNO.

FIG. 37 graphically depicts the patient population of the clinical study described at Example 1. All patients randomized received treatment. Discontinuations from the study were low. There were 395 patients (95.9%) in the post-treatment follow-up period.

FIG. 38A-FIG. 38D graphically depict the effect of SAR440340 on blood eosinophil levels. Data is presented for median (FIG. 38A) and mean (FIG. 38B) percent change of eosinophils in former smokers and median (FIG. 38C) and mean (FIG. 38D) percent change of eosinophils in current smokers.

FIG. 39A-FIG. 39B graphically depict the effect of SAR440340 on pre-BD FEV1. Data is presented for former smokers (FIG. 39A) and current smokers (FIG. 39B), showing that among former smokers SAR440340 improved pre-BD FEV1 by 90 mL.

FIG. 40A-FIG. 40B depict mean change in blood eosinophils in former smokers vs. current smokers, respectively. A similar affect was observed in both groups, but a larger effect was seen in former smokers.

FIG. 41A-FIG. 41B depict mean change in neutrophils in former smokers vs. current smokers, respectively.

FIG. 42A-FIG. 42B depict mean change in total IL-33 in former smokers vs. current smokers, respectively.

FIG. 43A-FIG. 43B depict mean change in pre-bronchodilator (pre-BD) FeNO in former smokers vs. current smokers, respectively.

FIG. 44A-FIG. 44B depict mean change in post-bronchodilator (post-BD) FeNO in former smokers vs. current smokers, respectively.

FIG. 45A-FIG. 45B depict percent change in the overall population and by smoker subgroup. 45A depicts pre-BD FEV1. 45B depicts post-BD FEV1.

FIG. 46 graphically depicts percent reduction of moderate-to-severe and severe AECOPD during the core and post-treatment periods, final data.

FIG. 47 graphically depicts percent reduction of moderate-to-severe AECOPD and effects on pre-BD during the core and post-treatment periods, final data.

FIG. 48A-FIG. 48B depict post-BD FEV1 (FIG. 48A) and pre-BD FVC (FIG. 48B) change for core and post-treatment period in the overall ITT population.

FIG. 49A-FIG. 49B depict pre-BD FEV1 in the core and post-treatment periods for former and current smokers, respectively.

FIG. 50A-FIG. 50B depict post-BD FEV1 in the core and post-treatment periods for former and current smokers, respectively.

FIG. 51 graphically depicts PK/PD during the core and post-treatment periods by smoking subgroup.

FIG. 52 graphically depicts blood eosinophil levels during the core and post-treatment periods by smoking subgroup.

FIG. 53 graphically depicts AECOPD-related clinical outcomes in former smokers during the core treatment period.

FIG. 54 summarizes results for select primary and secondary efficacy endpoints: modified intent-to-treat (mITT); mITT with a baseline eosinophil level of greater than or equal to 250 mm³; mITT with a baseline eosinophil level of less than 250 mm³; former smokers; and current smokers.

FIG. 55 graphically depicts time to first AECOPD in an mITT population.

FIG. 56 graphically depicts time to first AECOPD in former smokers (left panel) and current smokers (right panel).

FIG. 57 graphically depicts change from baseline in pre-BD FEV1 in an mITT population. Red shading, endpoint: mean weeks 26-24. Grey shading, variable treatment period weeks 24-52. Due to the variable treatment period, not all patients received after week 24, which is reflected in the number of patients at each time point.

FIG. 58 graphically depicts change from baseline in pre-BD FEV1 in former smokers in an mITT population. Red shading, endpoint: mean weeks 26-24. Grey shading, variable treatment period weeks 24-52. Due to the variable treatment period, not all patients received after week 24, which is reflected in the number of patients at each time point.

FIG. 59 graphically depicts lung function over time in current smokers as a change from baseline in pre-BD FEV1 in an mITT population. Red shading, endpoint: mean weeks 26-24. Grey shading, variable treatment period weeks 24-52. Due to the variable treatment period, not all patients received after week 24, which is reflected in the number of patients at each time point.

FIG. 60 summarizes post-BD FEV1 results at week 24 (mITT, baseline eosinophils <250 or ≥250/mm3, former/current smokers).

FIG. 61 graphically depicts lung function over time in an mITT population.

FIG. 62A-FIG. 62B depict lung function over time (post-BD FEV1) in (FIG. 62A) former smokers and (FIG. 62B) current smokers. Red shading, endpoint: mean weeks 26-24. Grey shading, variable treatment period weeks 24-52. Due to the variable treatment period, not all patients received after week 24, which is reflected in the number of patients at each time point.

FIG. 63 graphically depicts the mean change from baseline in blood eosinophil count (10⁹/mL), in a safety population.

FIG. 64 graphically depicts the percent change in pre-BD FEV1 and post-BD FEV1 in the overall population and by smoker subgroup.

FIG. 65 graphically depicts a comparison of PK and FEV1 in the ITT population.

FIG. 66 graphically depicts a comparison of EOS and FEV1 in the ITT population.

FIG. 67A-FIG. 67D depict genetic association results for the rare splice-acceptor variant rs146597587 in IL33. The rs146597587:C allele was associated with (FIG. 67A) a 46% reduction in total IL-33 protein levels in serum (N=437; P=7×10-39); (FIG. 67B) a reduction of 0.26 standard deviation (SD) units in peripheral blood eosinophil counts (N=549,261; meta-analysis P=6.3×10-84); (FIG. 67C) a 39% reduction in asthma risk (68,019 cases and 335,065 controls; meta-analysis P=1.7×10-20); and (FIG. 67D) a 21% reduction in COPD risk (22,352 cases and 335,065 controls; meta-analysis P=0.0049). CI denotes confidence interval, COPD chronic obstructive pulmonary disease, GHS Geisinger Health Service, OR odds ratio, SD standard deviation, SE standard error, and UKB UK Biobank study.

FIG. 68 shows the association between a common regulatory variant in IL33 (rs992969; effect allele: G) and risk of asthma and COPD in the UK Biobank and GHS studies.

FIG. 69 shows Mendelian randomization (MR) analysis between soluble IL-33 receptor (sIL-33R) levels and risk of asthma and COPD.

FIG. 70 depicts aggregate association between two common regulatory variants in IL33 (rs992969) and IL1RL1 (rs420101; effect allele: T) and risk of asthma and COPD in the UK Biobank and GHS Studies. The aggregate effect was estimated by testing the association between a genetic risk score (GRS, defined for each individual as the total number of minor alleles across the two variants; range 0 to 4) and disease case-control status, using logistic regression. GRS was expressed as a quantitative trait (range 0 to 4; trend test), and also as a binary trait, comparing individuals with GRS of 1 vs GRS of 0, GRS of 2 vs. GRS of 0, and GRS of 3 or 4 vs. GRS of 0. CI denotes confidence interval, COPD chronic obstructive pulmonary disease, GHS Geisinger Health Service, OR odds ratio, and UKB UK Biobank study.

FIG. 71 schematically depicts the study design showing former smokers according to the AERIFY-1 and AERIFY-2 (former smoker cohort) Phase 3 study design. SC, subcutaneous; Q2W, every two weeks; Q4W, every four weeks; ICS, inhaled corticosteroids; LABA, long-acting β2 adrenergic agonist, LAMA long-acting muscarinic antagonist.

FIG. 72 schematically depicts the AERIFY-2 Phase 3 study design showing the current smoker cohort. SC, subcutaneous; Q2W, every two weeks; ICS, inhaled corticosteroids; LABA, long-acting β2 adrenergic agonist, LAMA long-acting muscarinic antagonist.

### DETAILED DESCRIPTION

Before the invention is described, it is to be understood that this invention is not limited to particular methods and experimental conditions described, as such methods and conditions may vary. It is also to be understood that the terminology used herein is for the purpose of describing particular embodiments only, and is not intended to be limiting, because the scope of the invention will be limited only by the appended claims.

Unless defined otherwise, all technical and scientific terms used herein have the same meaning as commonly understood by one of ordinary skill in the art to which this invention belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

As used herein, the terms “treat,” “treating,” or the like, mean to alleviate symptoms, eliminate the causation of symptoms either on a temporary or permanent basis, or to prevent or slow the appearance of symptoms of the named disorder or condition (e.g., to prevent exacerbation of one or more symptoms of COPD).

Although any methods and materials similar or equivalent to those described herein can be used in the practice of the invention, the typical methods and materials are now described. All publications mentioned herein are incorporated herein by reference in their entirety.

### Methods for Reducing the Incidence of COPD Exacerbations

Methods for reducing the incidence of one or more COPD exacerbations in a subject in need thereof comprising administering a pharmaceutical composition comprising an interleukin-33 (IL-33) antagonist are provided. According to certain embodiments, the IL-33 antagonist is an antibody or antigen-binding fragment thereof that specifically binds IL-33. Exemplary anti-IL-33 antibodies that can be used in the context of the methods featured in the invention are described herein.

In one aspect, a subject is identified as having “mild,” “moderate,” “severe,” or “very severe” COPD if the subject receives such a diagnosis from a physician, based on the Global Initiative for Chronic Obstructive Lung Disease (GOLD) (Global strategy for the diagnosis, management, and prevention of chronic obstructive pulmonary disease (2017 report) (Available from the website: goldcopd.org/wp-content/uploads/2016/12/wms-GOLD-2017-Pocket-Guide.pdf)). In these aspects, a subject's COPD is classified based on airway limitation severity as tested using post-bronchodilator FEV1. A subject's COPD is classified as “mild” using the GOLD classification system if the subject's FEV1 is greater than or equal to 80% of the predicted FEV1. A predicted value for FEV1 is based on the FEV1 value for an average person of similar age, race, height, and gender with healthy lungs. A subject's COPD is classified as “moderate” on the GOLD classification system if the subject's FEV1 is greater than or equal to 50% of the predicted FEV1 but less than 80% of the predicted FEV1. A subject's COPD is classified as “severe” on the GOLD classification system if the subject's FEV1 is greater than or equal to 30% of the predicted FEV₁ but less than 50% of the predicted FEV1. A subject's COPD is classified as “very severe” on the GOLD classification system if the subject's FEV1 is less than 30% of the predicted FEV1.

In another aspect, methods for reducing the incidence or recurrence of COPD, or a COPD exacerbation, in a subject in need thereof are provided comprising administering a pharmaceutical composition comprising an IL-33 antagonist. A pharmaceutical composition comprising an IL-33 antagonist is provided for use to reduce the incidence or recurrence of COPD, or a COPD exacerbation in a subject in need thereof. As used herein, the expression “COPD exacerbation” means an increase in the severity and/or frequency and/or duration of one or more symptoms or indicia of COPD. A “COPD exacerbation” also includes any deterioration in the respiratory health of a subject that requires and or is treatable by a therapeutic intervention COPD (such as, e.g., steroid treatment, antibiotic treatment, inhaled corticosteroid treatment, hospitalization, etc.). In some embodiments, moderate exacerbations are defined as AECOPD events that require either systemic corticosteroids (such as intramuscular, intravenous or oral) and/or treatment with antibiotics. In some embodiments, severe exacerbations are defined as AECOPD events requiring hospitalization, emergency medical care visit, or resulting in death. According to certain embodiments, the annualized rate of moderate-to-severe acute exacerbations of COPD (AECOPD) includes moderate exacerbations and severe exacerbations.

A “reduction in the incidence or recurrence” of a COPD exacerbation means that a subject who has received the pharmaceutical compositions of the present invention experiences fewer COPD exacerbations (i.e., at least one fewer exacerbation) after treatment than before treatment, or experiences no COPD exacerbations for at least 4 weeks (e.g., 4, 6, 8, 12, 14, or more weeks) following initiation of treatment with a pharmaceutical composition of the present invention. A “reduction in the incidence or recurrence” of a COPD exacerbation alternatively means that, following administration of a pharmaceutical composition of the present invention, the likelihood that a subject experiences a COPD exacerbation is decreased by at least 10% (e.g., 10%, 15%, 20%, 25%, 30%, 35%, 40%, 45%, 50%, or more) as compared to a subject who has not received a pharmaceutical composition of the present invention.

Methods for reducing the incidence of COPD exacerbations in a subject in need thereof are provided comprising administering a pharmaceutical composition comprising an IL-33 antagonist to the subject as well as administering to the subject one or more maintenance doses of a second or a second and third controller, e.g., a long-acting beta-agonist (LABA), a long acting muscarinic antagonist (LAMA), and/or inhaled corticosteroid (ICS). A pharmaceutical composition comprising an IL-33 antagonist is provided for use, in combination with one or more maintenance doses of a second or a second and third controller, e.g., a long-acting beta-agonist (LABA), a long acting muscarinic antagonist (LAMA), and/or inhaled corticosteroid (ICS), to reduce the incidence of COPD exacerbations in a subject in need thereof. A combination of a pharmaceutical composition comprising an IL-33 antagonist and one or more maintenance doses of a second or a second and third controller, e.g., a long-acting beta-agonist (LABA), a long acting muscarinic antagonist (LAMA), and/or inhaled corticosteroid (ICS), is provided for use to reduce the incidence of COPD exacerbations in a subject in need thereof.

Suitable LABAs include, but are not limited to, salmeterol (e.g., Serevent®), formoterol (e.g., Foradil®, Perforomist®), indacaterol (e.g., Arcapta®), arformoterol (e.g., Brovana®), olodaterol (e.g., Stiverdi®), and the like.

Suitable ICSs include, but are not limited to, fluticasone (e.g., fluticasone propionate, e.g., Flovent®), budesonide, mometasone (e.g., mometasone furoate, e.g., Asmanex®), flunisolide (e.g., Aerobid®), dexamethasone acetate/phenobarbital/theophylline (e.g., Azmacort®), beclomethasone dipropionate HFA (Qvar®), and the like.

Suitable LAMAs include, but are not limited to, tiotropium bromide (e.g., Spiriva®), aclidinium bromide (e.g., Eklira®, Tudorza®), glycopyrronium bromide (e.g., Seebri®), umeclidinium (e.g., Incruse®) and the like.

Suitable LAMA and LABA combinations include, but are not limited to, umeclidinium and vilanterol (e.g., Anoro), olodaterol and tiotropium (e.g., Stiolto), indacaterol and glycopyrrolate (e.g., Utibron), and glycopyrrolate and formoterol (e.g., Bevespi).

Methods for reducing the incidence of COPD exacerbations in a subject in need thereof are provided comprising administering a pharmaceutical composition comprising an IL-33 antagonist to the subject as well as administering to the subject one or more reliever medications to eliminate or reduce one or more COPD-associated symptoms. A pharmaceutical composition comprising an IL-33 antagonist is provided for use, in combination with one or more reliever medications to eliminate or reduce one or more COPD-associated symptoms, to reduce the incidence of COPD exacerbations in a subject in need thereof. A combination comprising a pharmaceutical composition comprising an IL-33 antagonist and one or more reliever medications to eliminate or reduce one or more COPD-associated symptoms, is provided for use to reduce the incidence of COPD exacerbations in a subject in need thereof. Suitable reliever medications include, but are not limited to, quick-acting beta2-adrenergic receptor agonists such as, e.g., albuterol/salbutamol or levalbuterol/levosalbutamol (including ipratropium or ipratropium/short-acting Ragonists (SABA) combinations).

### Methods for Improving COPD-Associated Parameters

Methods for improving one or more COPD-associated parameters (also referred to herein as “COPD modifying” or “disease modifying”) in a subject in need thereof are provided, wherein the methods comprise administering a pharmaceutical composition comprising an IL-33 antagonist to the subject. A pharmaceutical composition comprising an IL-33 antagonist is provided for use to improve one or more COPD-associated in a subject in need thereof. A reduction in the incidence of an COPD exacerbation (as described above) may correlate with an improvement in one or more COPD-associated parameters; however, such a correlation is not necessarily observed in all cases.

Examples of “COPD-associated parameters” include, but are not limited to, one or any combination of: (1) annualized rate of moderate-to-severe AECOPD; (2) annualized rate of severe AECOPD; (3) relative absolute change from baseline (e.g., week 52) in forced expiratory volume in 1 second (FEV1) pre-bronchodilator (4) relative absolute change from baseline (e.g., week 24) in forced expiratory volume in 1 second (FEV1) pre-bronchodilator; (5) relative absolute change from baseline (e.g., week 52) in forced expiratory volume in 1 second (FEV1) post-bronchodilator; (6) relative percent change from baseline (e.g., at week 24 and 52) in forced expiratory volume in 1 second (FEV1) pre-bronchodilator; (7) relative percent change from baseline (e.g., at week 24) in forced expiratory volume in 1 second (FEV1) post-bronchodilator; (8) relative rate of decline (e.g., slope) in forced expiratory volume in 1 second (FEV1) pre- and/or post-bronchodilator; (9) time to first moderate or severe AECOPD; (10) change from baseline in the Exacerbation of COPD Tool (EXACT) scores (e.g., at week 24); (11) change from baseline in the Evaluating Respiratory Symptoms in COPD (E-RS) scores (e.g., at week 24); (12) change from baseline in the St. George Respiratory Questionnaire (SGRQ) scores (e.g., at week 24); (13) change from baseline in the Euro Quality of Life 5-Dimension Questionnaire (EQ-5D) scores (e.g., at week 24); (14) rate of moderate-to-severe AECOPD; (15) change from baseline to week 16-24 in forced vital capacity (FVC); (16) change from baseline in Modified British Medical Research Council Questionnaire (mMRC) scores (e.g., at week 24); (17) change from baseline in the Health-Related Quality of Life Questionnaire (HRQOL) scores (e.g., at week 24); (18) change from baseline in Body mass index, airflow Obstruction, Dyspnea, Exercise performance (BODE) scores (e.g., at week 24); (19) change from baseline in daily steps (e.g., at week 24); (20) days on oral corticosteroids; (21) days on antibiotics; (22) change from baseline in resting oxygen saturation (e.g., at week 24); (23) change from baseline in resting respiratory rate (e.g., at week 24); (24) maintenance of lung function (e.g., relative to no treatment or to treatment with placebo); and (25) reduction in lung function decline (e.g., relative to no treatment or to treatment with placebo).

An “improvement in an COPD-associated parameter” means an increase from baseline in FEV1 or time to first moderate or severe AECOPD, and/or a decrease from baseline rate of AECOPD. As used herein, the term “baseline,” with regard to a COPD-associated parameter, means the numerical value of the COPD-associated parameter for a patient prior to or at the time of administration of a pharmaceutical composition comprising an IL-33 antagonist.

To determine whether an COPD-associated parameter has “improved,” the parameter is quantified at baseline and at a time point after administration of the pharmaceutical composition described herein. For example, an COPD-associated parameter may be measured at day 1, day 2, day 3, day 4, day 5, day 6, day 7, day 8, day 9, day 10, day 11, day 12, day 14, or at week 3, week 4, week 5, week 6, week 7, week 8, week 9, week 10, week 11, week 12, week 13, week 14, week 15, week 16, week 17, week 18, week 19, week 20, week 21, week 22, week 23, week 24, or longer, after the initial treatment with the pharmaceutical composition. The difference between the value of the parameter at a particular time point following initiation of treatment and the value of the parameter at baseline is used to establish whether there has been an “improvement” in the COPD associated parameter (e.g., an increase or decrease, as the case may be, depending on the specific parameter being measured).

The terms “acquire” or “acquiring” as used herein, refer to obtaining possession of a physical entity, or a value, e.g., a numerical value, by “directly acquiring” or “indirectly acquiring” the physical entity or value, such as an COPD-associated parameter. “Directly acquiring” means performing a process (e.g., performing a synthetic or analytical method) to obtain the physical entity or value. “Indirectly acquiring” refers to receiving the physical entity or value from another party or source (e.g., a third-party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”).

Information that is acquired indirectly can be provided in the form of a report, e.g., supplied in paper or electronic form, such as from an online database or application (an “App”). The report or information can be provided by, for example, a healthcare institution, such as a hospital or clinic; or a healthcare provider, such as a doctor or nurse.

Forced Expiratory Volume in 1 Second (FEV1). According to certain embodiments, administration of an IL-33 antagonist to a patient results in an increase from baseline of forced expiratory volume in 1 second (FEV1). Methods for measuring FEV1 are known in the art. For example, a spirometer that meets the 2005 American Thoracic Society (ATS)/European Respiratory Society (ERS) recommendations can be used to measure FEV1 in a patient. The ATS/ERS Standardization of Spirometry may be used as a guideline. Spirometry is generally performed between 6 and 10 AM after an albuterol withhold of at least 6 hours. Pulmonary function tests are generally measured in the sitting position, and the highest measure is recorded for FEV1 (in liters).

The disclosure includes therapeutic methods that result in an increase of FEV1 from baseline of at least 0.01 L at week 24 following initiation of treatment with a pharmaceutical composition comprising an anti-L-33 antagonist. The disclosure includes a pharmaceutical composition comprising an anti-L-33 antagonist for use to increase FEV1 from baseline of at least 0.01 L at week 24 following initiation of treatment with said pharmaceutical composition. For example, administration of an IL-33 antagonist causes an increase of FEV1 from baseline of about 0.01 L, 0.02 L, 0.03 L, 0.04 L, 0.05 L, 0.10 L, 0.12 L, 0.14 L, 0.16 L, 0.18 L, 0.20 L, 0.22 L, 0.24 L, 0.26 L, 0.28 L, 0.30 L, 0.32 L, 0.34 L, 0.36 L, 0.38 L, 0.40 L, 0.42 L, 0.44 L, 0.46 L, 0.48 L, 0.50 L, or more at week 24.

Forced Vital Capacity (FVC). According to certain embodiments, administration of an IL-33 antagonist to a patient results in an increase from baseline of FVC (forced vital capacity). Methods for measuring FVC are known in the art. For example, a spirometer that meets the 2005 American Thoracic Society (ATS)/European Respiratory Society (ERS) recommendations can be used to measure FVC in a patient. The ATS/ERS Standardization of Spirometry may be used as a guideline. Spirometry is generally performed between 6 and 10 AM after an albuterol withhold of at least 6 hours. Pulmonary function tests are generally measured in the sitting position, and the highest measure is recorded for FVC (in liters).

FEF25-75%. According to certain embodiments, administration of an IL-33 antagonist to a patient results in an increase from baseline of FEF25-75% (forced expiratory flow between 25% and 75%). Methods for measuring FEF are known in the art. For example, a spirometer that meets the 2005 American Thoracic Society (ATS)/European Respiratory Society (ERS) recommendations can be used to measure FEV1 in a patient. The FEF25-75% is the speed (in liters per second) at which a person can empty the middle half of his or her air during a maximum expiration (i.e., Forced Vital Capacity or FVC). The parameter relates to the average flow from the point at which 25 percent of the FVC has been exhaled to the point at which 75 percent of the FVC has been exhaled. The FEF25-75% of a subject provides information regarding small airway function, such that the extent of small airway disease and/or inflammation. A change in FEF25-75% is an early indicator of obstructive lung disease. In certain embodiments, an improvement and/or increase in the FEF25-75% parameter is an improvement of at least 10%, 25%, 50% or more as compared to baseline. In certain embodiments, the methods of the invention result in normal FEF25-75% values in a subject (e.g., values ranging from 50-60% and up to 130% of the average).

The disclosure includes therapeutic methods that result in a decrease in AECOPD from baseline of at least 5% at week 24 following initiation of treatment with a pharmaceutical composition comprising an anti-IL-33 antagonist. The disclosure includes a pharmaceutical composition comprising an anti-IL-33 antagonist for use to decrease AECOPD from baseline of at least 5% at week 24 following initiation of treatment with said pharmaceutical composition. For example, according to the invention, administration of an IL-33 antagonist to a subject in need thereof causes a decrease in AECOPD from baseline of about 5%, about 10%, about 15%, about 20%, about 25%, about 30%, about 35%, about 40%, about 45%, about 50%, about 55%, about 60%, about 65%, about 70%, or more at week 24.

The disclosure includes therapeutic methods that result in a reduction in the probability of first AECOPD at a specific time point of at least 5% at week 24 following initiation of treatment with a pharmaceutical composition comprising an anti-IL-33 antagonist versus baseline. The disclosure includes a pharmaceutical composition comprising an anti-IL-33 antagonist for use to reduce the probability of first AECOPD at a specific time point of at least 5% at week 24 following initiation of treatment with said pharmaceutical composition. For example, according to the invention, administration of an IL-33 antagonist to a subject in need thereof causes a reduction in the probability of first AECOPD at a specific time point of about 5%, about 10%, about 15%, about 20%, about 25%, about 30%, about 35%, about 40%, about 45%, about 50%, about 55%, about 60%, about 65%, about 70%, or more at week 24 versus baseline.

Albuterol/Levalbuterol Use. According to certain embodiments, administration of an IL-33 antagonist to a patient results in a decrease from baseline of daily albuterol or levalbuterol use. The number of albuterol/levalbuterol inhalations can be recorded daily by the patients in a diary, PEF meter, or other recording device. During treatment with the pharmaceutical composition described herein, use of albuterol/levalbuterol typically may be on an as-needed basis for symptoms, not on a regular basis or prophylactically. The baseline number of albuterol/levalbuterol inhalations/day may be calculated based on the mean for the 7 days prior to administration of the first dose of pharmaceutical composition comprising the IL-33 antagonist.

The invention includes therapeutic methods that result in a decrease in albuterol/levalbuterol use from baseline of at least 0.25 puffs per day at week 12 following initiation of treatment with a pharmaceutical composition comprising an anti-IL-33 antagonist. For example, administration of an IL-33 antagonist to a subject in need thereof causes a decrease in albuterol/levalbuterol use from baseline of about 0.25 puffs per day, 0.50 puffs per day, 0.75 puffs per day, 1.00 puff per day, 1.25 puffs per day, 1.5 puffs per day, 1.75 puffs per day, 2.00 puffs per day, 2.25 puffs per day, 2.5 puffs per day, 2.75 puffs per day, 3.00 puffs per day, or more at week 12.

Daily Steps. According to certain embodiments, administration of an IL-33 antagonist to a patient results in a change from baseline in daily steps, e.g., results in an increase in daily steps over a defined period of time relative to daily steps over a defined period of time prior to administration of the IL-33 antagonist.

Corticosteroid/Antibiotic Use. According to certain embodiments, administration of an IL-33 antagonist to a patient results in a reduction of days on oral corticosteroids. According to certain embodiments, administration of an IL-33 antagonist to a patient results in a reduction of days on antibiotics over a defined period of time relative to number of days the patient was on antibiotics over a defined period of time prior to administration of the IL-33 antagonist.

Oxygen Saturation. In some embodiments, administration of an IL-33 antagonist to a patient results in a change from baseline in resting oxygen saturation, e.g., results in increased resting oxygen saturation than is obtained prior to administration of the IL-33 antagonist.

Respiratory Rate. In some embodiments, administration of an IL-33 antagonist to a patient results in a change from baseline in resting respiratory rate, e.g., a decrease or an increase in respiratory rate. In certain exemplary embodiments, administration of an IL-33 antagonist to a patient results in a decrease from baseline in resting respiratory rate relative to resting respiratory rate prior to administration of the IL-33 antagonist.

Body Mass Index, Airflow Obstruction, Dyspnea, Exercise Performance (BODE) Index. According to certain embodiments, administration of an IL-33 antagonist to a patient results in an improvement from baseline of BODE index score. In some embodiments, administration of an IL-33 antagonist to a patient results in an improvement from baseline of BODE index score of greater than 1 point. The BODE index integrates body mass index, airflow limitation (FEV1), dyspnea and 6-minute walk distance, and predicts mortality in COPD patients. (Celli, et al. The Body Mass Index, Airflow Obstruction, Dyspnea, Exercise Performance (BODE) index in chronic obstructive pulmonary disease. New Eng. J. Med. 2004; 350:1005-1012.)

COPD Assessment Test (CAT) Score. According to certain embodiments, administration of an IL-33 antagonist to a patient results in a decrease from baseline of CAT score. An anti-IL-33 antagonist is provided for use in a patient to decrease from baseline CAT score. The CAT is a questionnaire that is designed for patients with COPD to measure the effects of the disease on their quality of lives (COPD assessment test. Available from the website: catestonline.org/). The CAT is an 8-item self-administered questionnaire which has been developed for use in routine clinical practice to measure the health status of patients with COPD. The CAT score ranges from 0 to 40, a higher score indicating a higher impact on health status. The test is about cough, phlegm, chest tightness, dyspnea, activity limitation, confidence, sleep and energy. Patients score questions from 1-5 according to their own feelings about the disease (1=I am very happy; 5=I am very sad).

St. George's Respiratory Questionnaire (SGRQ). According to certain embodiments, administration of an IL-33 antagonist to a patient results in a decrease from baseline of SGRQ score. An anti-IL-33 antagonist is provided for use in a patient to decrease from baseline SGRQ score. The St. George's Respiratory Questionnaire (SGRQ) is a 50-item questionnaire designed to measure and quantify health-related health status in adult patients with chronic airflow limitation (Jones et al. A self-complete measure of health status for chronic airflow limitation. The St. George's Respiratory Questionnaire. Am Rev Respir Dis. 1992 June; 145(6):1321-7). A global score ranges from 0 to 100. Scores by dimension are calculated for three domains: Symptoms, Activity and Impacts (Psycho-social) as well as a total score. Lower score indicates better quality of life (QoL). The first part (“symptoms”) evaluates symptomatology, including frequency of cough, sputum production, wheeze, breathlessness and the duration and frequency of attacks of breathlessness or wheeze. The second part has two components: “activity” and “impacts.” The “activity” section addresses activities that cause breathlessness or are limited because of breathlessness. The “impacts” section covers a range of factors including influence on employment, being in control of health, panic, stigmatization, the need for medication, side effects of prescribed therapies, expectations for health and disturbances of daily life. The recall period of the questionnaire is over the past 4 weeks. Psychometric testing has demonstrated its repeatability, reliability and validity. Sensitivity has been demonstrated in clinical trials. A minimum change in score of 4 units was established as clinically relevant after patient and clinician testing. The SGRQ has been used in a range of disease groups including asthma, COPD and bronchiectasis.

Exacerbations of chronic obstructive pulmonary disease tool (EXACT). According to certain embodiments, administration of an IL-33 antagonist to a patient results in a decrease from baseline of EXACT score. An anti-IL-33 antagonist is provided for use in a patient to decrease from baseline EXACT score. The EXACT Total Score measures symptoms of acute bacterial exacerbations of chronic bronchitis-COPD (ABECB-COPD), i.e., an acute, sustained, and worsening of signs and symptoms beyond day-to-day variability. The instrument's total score is made up of a total of 14 items representing the following domains: breathlessness (5 items), cough and sputum (2 items), chest symptoms (3 items), difficulty bringing up sputum (1 item), tired or weak (1 item), sleep disturbance (1 item), and scared or worried (1 item). The EXACT is a daily diary, completed each evening before bedtime. The instrument was developed with e-diary administration in mind, with cognitive interviews performed with paper pen booklet and personal digital assistant (PDA) to document respondent understanding in either mode and user acceptance of the PDA.

Evaluating Respiratory Symptoms in COPD (E-RS). According to certain embodiments, administration of an IL-33 antagonist to a patient results in a patient reporting better health in Evaluating Respiratory Symptoms in COPD (E-RS). An anti-IL-33 antagonist is provided for use in a patient to have this patient reporting better health in E-RS. The E-RS scale was designed to serve as a primary, secondary, or exploratory endpoint in clinical trials evaluating the effect of treatment on respiratory symptoms of COPD. The E-RS is based on the 11 respiratory symptom items from the 14-item EXACT, a daily diary used to measure exacerbations of COPD. The E-RS yields a total score, quantifying respiratory symptom severity overall, and 3 subscale scores assessing breathlessness; cough and sputum; and chest symptoms. This permits two validated uses for a single diary: quantification of respiratory symptoms in stable COPD using E-RS total and subscale scores and the assessment of acute exacerbations (frequency, severity, duration of symptom-defined events, and change in exacerbation symptoms with medically-treated events) using the EXACT total score.

EuroQual questionnaire (EQ-5D-3 L or EQ-5D-5 L). According to certain embodiments, administration of an IL-33 antagonist to a patient results in a patient reporting better health in a EuroQual questionnaire (EQ-5D-3 L or EQ-5D-5 L). An anti-IL-33 antagonist is provided for use in a patient to have this patient reporting better health in a EuroQual questionnaire (EQ-5D-3 L or EQ-5D-5 L). The EQ-5D-5 L and EQ-5D-3 L are standardized health-related QoL questionnaires developed by the EuroQol Group in order to provide a simple, generic measure of health for clinical and economic appraisal.

Modified British Medical Research Council Questionnaire (mMRC). According to certain embodiments, administration of an IL-33 antagonist to a patient results in a patient reporting better health status in a Modified British Medical Research Council Questionnaire (mMRC). An anti-IL-33 antagonist is provided for use in a patient to have this patient reporting better health status in a Modified British Medical Research Council Questionnaire (mMRC). The Modified British Medical Research Council Questionnaire (mMRC) is a questionnaire that assesses breathlessness. (Fletcher et al. Standardised questionnaire on respiratory symptoms: a statement prepared and approved by the MRC Committee on the Aetiology of Chronic Bronchitis (MRC breathlessness score). BMJ1960; 2: 1662.)

Health-Related Quality of Life (HRQOL) Questionnaire. According to certain embodiments, administration of an IL-33 antagonist to a patient results in a patient reporting better health status in a Health-Related Quality of Life (HRQOL) Questionnaire. Centers for Disease Control and Prevention. Measuring Healthy Days. Atlanta, Ga.: CDC, November 2000, Available at the website: cdc.gov/hrqol/pdfs/mhd.pdf.) An anti-IL-33 antagonist is provided for use in a patient to have this patient reporting better health status in a HRQOL Questionnaire.

Biomarkers. In certain embodiments, the subject experiences an improvement in lung function as measured by a biomarker. In certain exemplary embodiments, a subject experiences an increase in a biomarker level after administration of anti-IL-33 antagonist (relative to the biomarker level before administration of the anti-IL-33 antagonist). In certain exemplary embodiments, a subject experiences a decrease in a biomarker level after administration of anti-IL-33 antagonist (relative to the biomarker level before administration of the anti-IL-33 antagonist). For example, the biomarker may be selected from the group consisting of blood eosinophils, blood neutrophils, fractional exhaled nitric oxide (FeNO) (e.g., pre-bronchodilator FeNO), total IL-33, soluble IL-33 receptor (sST2), calcitonin, pulmonary and activation-regulated chemokine (PARC), blood C-reactive protein (CRP), blood IL-6, eotaxin-3, total IgE, fibrinogen, calcitonin, procalcitonin, calcitonin gene-related peptide (CGRP), resistin-like alpha (RETNA), chemokine (CC motif) ligand 8 (Ccl8), serum amyloid A3 (Saa3), Gm1975 (BC117090), killer cell lectin-like receptor (Kirgl), stefin A1 (Csta), membrane-spanning 4-domain (Ms4a8a), chemokine (C—C motif) ligand 11 (Ccl11), serine (or cysteine) peptides (Serpina3f), and the like. In certain embodiments, whole blood mRNA samples are obtained for sequencing or whole transcriptome analysis. In certain embodiments, serum and/or plasma samples are obtained and optionally archived for research regarding exploratory biomarkers of disease or drug effect. In certain embodiments, samples are used for research to develop methods, assays, prognostics and/or companion diagnostics related to IL-33, disease process(es), pathways associated with disease state and/or mechanism of action of the study intervention. In certain embodiments, an improvement in lung function is indicated by a reduction or increase (as appropriate) at week 4, week 12 or week 24 following treatment.

### Methods for Treating COPD

In some embodiments, methods for treating COPD, including, e.g., moderate-to-severe COPD, in a subject in need thereof are provided, wherein the methods comprise administering a pharmaceutical composition comprising an IL-33 antagonist. In certain embodiments, the methods are useful for treating moderate-to-severe COPD in a subject. In certain embodiments, the methods are useful for reducing one or more AECOPD events. A pharmaceutical composition comprising an anti-IL-33 antagonist is provided to treat COPD, including, e.g., moderate-to-severe COPD, in a subject in need thereof. A pharmaceutical composition comprising an anti-IL-33 antagonist is provided to treat moderate-to-severe COPD in a subject. It is also provided a pharmaceutical composition comprising an anti-IL-33 antagonist to reduce one or more AECOPD events in a patient.

In one aspect, methods for treating COPD are provided comprising: (a) selecting a patient that exhibits a blood eosinophil level of equal to or greater than 300 cells per microliter; and (b) administering to the patient a pharmaceutical composition comprising an IL-33 antagonist. In one aspect of the composition for use, the patient exhibits a blood eosinophil level of equal to or greater than 300 cells per microliter.

In one aspect, methods for treating COPD are provided comprising: (a) selecting a patient that exhibits a blood eosinophil level of equal to or greater than 250 cells per microliter; and (b) administering to the patient a pharmaceutical composition comprising an IL-33 antagonist. In one aspect of the composition for use, the patient exhibits a blood eosinophil level of equal to or greater than 250 cells per microliter.

In one aspect, methods for treating COPD are provided comprising: (a) selecting a patient that exhibits a blood eosinophil level of less than 300 cells per microliter; and (b) administering to the patient a pharmaceutical composition comprising an IL-33 antagonist. In one aspect of the composition for use, the patient exhibits a blood eosinophil level of less than cells per microliter.

In another aspect, methods for treating COPD are provided comprising: (a) selecting a patient that exhibits a blood eosinophil level of 150-299 cells per microliter; and (b) administering to the patient a pharmaceutical composition comprising an IL-33 antagonist. In one aspect of the composition for use, the patient exhibits a blood eosinophil level of 150-299 cells per microliter.

In another aspect, methods for treating COPD are provided comprising: (a) selecting a patient that exhibits a blood eosinophil level of less than 150 cells per microliter; and (b) administering to the patient a pharmaceutical composition comprising an IL-33 antagonist. In one aspect of the composition for use, the patient exhibits a blood eosinophil level of less than 150 cells per microliter.

In a related aspect, methods for treating COPD comprising an add-on therapy to background therapy are provided. In a related aspect, an IL-33 antagonist is provided for use to treat COPD in a patient, wherein IL-33 antagonist is used as an add-on therapy to background therapy. In certain embodiments, an IL-33 antagonist is administered as an add-on therapy to a COPD patient who is on background therapy for a certain period of time (e.g., 1 week, 2 weeks, 3 weeks, 1 month, 2 months, 5 months, 12 months, 18 months, 24 months, or longer) (also called the “stable phase”). In certain embodiments, an IL-33 antagonist is provided for use to treat COPD in a patient, where IL-33 antagonist is administered as an add-on therapy to a COPD patient who is on background therapy for a certain period of time. In some embodiments, the background therapy comprises a ICS and a LABA. In other embodiments the background therapy comprises a ICS and a LAMA. In other embodiments the background therapy comprises a LABA and a LAMA. In other embodiments the background therapy comprises a ICS, a LAMA, and a LABA. In some embodiments, the background therapy comprises a PDE-4 inhibitor, such as roflumilast. In other embodiments, the background therapy comprises azithromycin.

In some embodiments, the invention includes a method for reducing a COPD patient's dependence on ICS, LAMA, or LABA for the treatment of one or more COPD exacerbations comprising: (a) selecting a patient who has moderate-to-severe COPD that is not well-controlled with a background therapy comprising an ICS, a LABA, a LAMA, or a combination thereof, and administering to the patient a pharmaceutical composition comprising an IL-33 antagonist. A pharmaceutical composition comprising an IL-33 antagonist is provided for use to reduce a COPD patient's dependence on ICS, LAMA, or LABA for the treatment of one or more COPD exacerbations, in a patient who has moderate-to-severe COPD that is not well-controlled with a background COPD therapy comprising an ICS, a LABA, a LAMA, or a combination thereof.

In some embodiments, the invention includes a method for the treatment of one or more COPD exacerbations in a patient that is using ICS, LAMA, or LABA chronically comprising: (a) selecting a patient who has moderate-to-severe COPD that is using ICS, a LABA, a LAMA, or a combination thereof chronically; and administering to the patient a pharmaceutical composition comprising an IL-33 antagonist. A pharmaceutical composition comprising an IL-33 antagonist is provided for use to treat one or more COPD exacerbations in a patient that is using ICS, LAMA, or LABA chronically, in a patient who has moderate-to-severe COPD that is using ICS, a LABA, a LAMA, or a combination thereof chronically.

### Interleukin-33 (IL-33) Antagonists

The methods featured in the invention comprise administering to a subject in need thereof a therapeutic composition comprising an IL-33 antagonist. As used herein, an “IL-33 antagonist” is any agent that binds to or interacts with IL-33 and inhibits the normal biological signaling function of IL-33 when IL-33 is expressed on a cell in vitro or in vivo.

Non-limiting examples of categories of IL-33 antagonists include small molecule IL-33 antagonists, anti-IL-33 aptamers, peptide-based IL-33 antagonists (e.g., “peptibody” molecules), and antibodies or antigen-binding fragments of antibodies that specifically bind human IL-33.

According to certain embodiments, the IL-33 antagonist comprises an anti-IL-33 antibody or antigen-binding fragment thereof that can be used in the context of the methods featured in the invention as described elsewhere herein. For example, in one embodiment, the IL-33 antagonist is an antibody or antigen-binding fragment thereof that specifically binds to an IL-33, and comprises the heavy chain and light chain (complementarity determining region) CDR sequences from the heavy chain variable region (HCVR) and light chain variable region (LCVR) of SEQ ID NOs: 2 and 10, respectively. In another embodiment, the IL-33 antagonist is an antibody or antigen-binding fragment thereof that specifically binds to an IL-33, and comprises the heavy chain and light chain CDR sequences of SEQ ID NOs: 4, 6 and 8 and SEQ ID NOs: 12, 14 and 16, respectively. In another embodiment, the IL-33 antagonist is an antibody or antigen-binding fragment thereof that specifically binds to an IL-33, and comprises an HCVR/LCVR pair of SEQ ID NOs: 2 and 10, respectively.

DNA sequence encoding SAR440340 (REGN3500) HCVR:
(SEQ ID NO: 1)
aggtgcagct ggtggagtct gggggaaact tggaacagcc
tggggggtcc cttagactct cctgtacagc ctctggattc
acctttagca gatctgccat gaactgggtc cgccgggctc
cagggaaggg gctggagtgg gtctcaggaa ttagtggtag
tggtggtcga acatactacg cagactccgt gaagggccgg
ttcaccatct ccagagacaa ttccaagaat acgctatatc
tgcaaatgaa cagcctgagc gccgaggaca cggccgcata
ttactgtgcg aaagattcgt atactaccag ttggtacgga
ggtatggacg tctggggcca cgggaccacg gtcaccgtct
cctca.
SAR440340 (REGN3500) HCVR amino acid sequence:
(SEQ ID NO: 2)
VQLVESGGNLEQPGGSLRLSCTASGFTFSRSAMNWVRRAPGKGLEWVSGI
SGSGGRTYYADSVKGRFTISRDNSKNTLYLQMNSLSAEDTAAYYCAKDSY
TTSWYGGMDVWGHGTTVTVSS.
DNA sequence encoding SAR440340 (REGN3500) HCDR1:
(SEQ ID NO: 3)
ggattcacctt tagcagatct gcc.
SAR440340 (REGN3500) HCDR1 amino acid sequence:
(SEQ ID NO: 4)
GFTFSRSA.
DNA sequence encoding SAR440340 (REGN3500) HCDR2:
(SEQ ID NO: 5)
attagtggtag tggtggtcga aca.
SAR440340 (REGN3500) HCDR2 amino acid sequence:
(SEQ ID NO: 6)
ISGSGGRT.
DNA sequence encoding SAR440340 (REGN3500) HCDR3:
(SEQ ID NO: 7)
gcgaaagattc gtatactacc agttggtacg gaggtatgga cgtc.
SAR440340 (REGN3500) HCDR3 amino acid sequence:
(SEQ ID NO: 8)
AKDSYTTSWYGGMDV.
DNA sequence encoding SAR440340 (REGN3500) LCVR:
(SEQ ID NO: 9)
acatccagat gacccagtct ccatcttccg tgtctgcatc
tgtaggagac agagtcacca tcacttgtcg ggcgagtcag
ggtattttca gctggttagc ctggtatcag cagaaaccag
gaaaagcccc taagctcctg atctatgctg cttccagttt
acaaagtggg gtcccatcaa gattcagcgg cagtggatct
gggacagatt tcactctcac catcagcagc ctgcagcctg
aggattttgc aatttactat tgtcaacagg ctaacagtgt
cccgatcacc ttcggccaag ggacacgact ggagattaaa
cga.
SAR440340 (RRGN3500) LCVR amino acid sequence:
(SEQ ID NO: 10)
IQMTQSPSSVSASVGDRVTITCRASQGIFSWLAWYQQKPGKAPKLLIYAA
SSLQSGVPSRFSGSGSGTDFTLTISSLQPEDFAIYYCQQANSVPITFGQG
TRLEIKR.
DNA sequence encoding SAR440340 (REGN3500) LCDR1:
(SEQ ID NO: 11)
cagggtatttt cagctgg.
SAR440340 (REGN3500) LCDR1 amino acid sequence:
(SEQ ID NO: 12)
QGIFSW.
DNA sequence encoding SAR440340 (REGN3500) LCDR2:
(SEQ ID NO: 13)
gctgcttcc.
SAR440340 (REGN3500) LCDR2 amino acid sequence:
(SEQ ID NO: 14)
AAS.
DNA sequence encoding SAR440340 (REGN3500) LCDR3:
(SEQ ID NO: 15)
caacaggctaa cagtgtcccg atcacc.
SAR440340 (REGN3500) LCDR3 amino acid sequence:
(SEQ ID NO: 16)
QQANSVPIT.
DNA sequence encoding SAR440340 (REGN3500) heavy
chain:
(SEQ ID NO: 17)
aggtgcagct ggtggagtct gggggaaact tggaacagcc
tggggggtcc cttagactct cctgtacagc ctctggattc
acctttagca gatctgccat gaactgggtc cgccgggctc
cagggaaggg gctggagtgg gtctcaggaa ttagtggtag
tggtggtcga acatactacg cagactccgt gaagggccgg
ttcaccatct ccagagacaa ttccaagaat acgctatatc
tgcaaatgaa cagcctgagc gccgaggaca cggccgcata
ttactgtgcg aaagattcgt atactaccag ttggtacgga
ggtatggacg tctggggcca cgggaccacg gtcaccgtct
cctcagcctc caccaagggc ccatcggtct tccccctggc
gccctgctcc aggagcacct ccgagagcac agccgccctg
ggctgcctgg tcaaggacta cttccccgaa ccggtgacgg
tgtcgtggaa ctcaggcgcc ctgaccagcg gcgtgcacac
cttcccggct gtcctacagt cctcaggact ctactccctc
agcagcgtgg tgaccgtgcc ctccagcagc ttgggcacga
agacctacac ctgcaacgta gatcacaagc ccagcaacac
caaggtggac aagagagttg agtccaaata tggtccccca
tgcccaccct gcccagcacc tgagttcctg gggggaccat
cagtcttcct gttcccccca aaacccaagg acactctcat
gatctcccgg acccctgagg tcacgtgcgt ggtggtggac
gtgagccagg aagaccccga ggtccagttc aactggtacg
tggatggcgt ggaggtgcat aatgccaaga caaagccgcg
ggaggagcag ttcaacagca cgtaccgtgt ggtcagcgtc
ctcaccgtcc tgcaccagga ctggctgaac ggcaaggagt
acaagtgcaa ggtctccaac aaaggcctcc cgtcctccat
cgagaaaacc atctccaaag ccaaagggca gccccgagag
ccacaggtgt acaccctgcc cccatcccag gaggagatga
ccaagaacca ggtcagcctg acctgcctgg tcaaaggctt
ctaccccagc gacatcgccg tggagtggga gagcaatggg
cagccggaga acaactacaa gaccacgcct cccgtgctgg
actccgacgg ctccttcttc ctctacagca ggctcaccgt
ggacaagagc aggtggcagg aggggaatgt cttctcatgc
tccgtgatgc atgaggctct gcacaaccac tacacacaga
agtccctctc cctgtctctg ggtaaatga.
SAR440340 (REGN3500) heavy chain amino acid
sequence:
(SEQ ID NO: 18)
VQLVESGGNLEQPGGSLRLSCTASGFTFSRSAMNWVRRAPGKGLEWVSGI
SGSGGRTYYADSVKGRFTISRDNSKNTLYLQMNSLSAEDTAAYYCAKDSY
TTSWYGGMDVWGHGTTVTVSSASTKGPSVFPLAPCSRSTSESTAALGCLV
KDYFPEPVTVSWNSGALTSGVHTFPAVLQSSGLYSLSSVVTVPSSSLGTK
TYTCNVDHKPSNTKVDKRVESKYGPPCPPCPAPEFLGGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK.
DNA sequence encoding SAR440340 (REGN3500) light
chain:
(SEQ ID NO: 19)
acatccagat gacccagtct ccatcttccg tgtctgcatc
tgtaggagac agagtcacca tcacttgtcg ggcgagtcag
ggtattttca gctggttagc ctggtatcag cagaaaccag
gaaaagcccc taagctcctg atctatgctg cttccagttt
acaaagtggg gtcccatcaa gattcagcgg cagtggatct
gggacagatt tcactctcac catcagcagc ctgcagcctg
aggattttgc aatttactat tgtcaacagg ctaacagtgt
cccgatcacc ttcggccaag ggacacgact ggagattaaa
cgaactgtgg ctgcaccatc tgtcttcatc ttcccgccat
ctgatgagca gttgaaatct ggaactgcct ctgttgtgtg
cctgctgaat aacttctatc ccagagaggc caaagtacag
tggaaggtgg ataacgccct ccaatcgggt aactcccagg
agagtgtcac agagcaggac agcaaggaca gcacctacag
cctcagcagc accctgacgc tgagcaaagc agactacgag
aaacacaaag tctacgcctg cgaagtcacc catcagggcc
tgagctcgcc cgtcacaaag agcttcaaca ggggagagtg
ttag.
SAR440340 (REGN3500) light chain amino acid
sequence:
(SEQ ID NO: 20)
IQMTQSPSSVSASVGDRVTITCRASQGIFSWLAWYQQKPGKAPKLLIYAA
SSLQSGVPSRFSGSGSGTDFTLTISSLQPEDFAIYYCQQANSVPITFGQG
TRLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC.

The term “human IL-33” (hIL-33) refers to a human cytokine that specifically binds to interleukin-33 receptor (IL-33R).

The term “antibody” refers to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2, and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments, the FRs of the anti-IL-33 antibody, or an antigen-binding portion thereof may be identical to the human germline sequences, or may be naturally or artificially modified. An amino acid consensus sequence may be defined based on a side-by-side analysis of two or more CDRs.

The term “antibody” also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds to an antigen to form a complex. Antigen-binding fragments of an antibody may be derived, e.g., from full antibody molecules using any suitable standard techniques, such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include, but are not limited to: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment.”

An antigen-binding fragment of an antibody will typically comprise at least one variable domain. The variable domain may be of any size or amino acid composition and will generally comprise at least one CDR that is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody described herein include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (X) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (Xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids that result in a flexible or semi-flexible linkage between adjacent variable and/or constant domains in a single polypeptide molecule, typically the hinge region may consist of between 2 to 60 amino acids, typically between 5 to 50, or typically between 10 to 40 amino acids. Moreover, an antigen-binding fragment of an antibody described herein may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, may be adapted for use in the context of an antigen-binding fragment of an antibody described herein using routine techniques available in the art.

The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

The term “human antibody” includes antibodies having variable and constant regions derived from human germline immunoglobulin sequences. The human antibodies featured in the invention may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody” does not include antibodies in which CDR sequences derived from the germline of another mammalian species, such as a mouse, have been grafted onto human framework sequences.

The term “recombinant human antibody” includes all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The invention encompasses antibodies having one or more mutations in the hinge, C_{H}2, or C_{H}3 region, which may be desirable, for example, in production, to improve the yield of the desired antibody form.

An “isolated antibody” means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” IL-33, as featured in the invention, includes antibodies that bind IL-33, respectively, or portion thereof, with a K_{D} of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM, or less than about 0.5 nM, as measured in a surface plasmon resonance assay. An isolated antibody that specifically binds human IL-33 may, however, have cross-reactivity to other antigens, such as IL-33 molecules from other (non-human) species.

The anti-IL-33 antibodies useful for the methods may comprise one or more amino acid substitutions, insertions, and/or deletions (e.g. 1, 2, 3, 4, 5, 6, 7, 8, 9, or 10 substitutions and/or 1, 2, 3, 4, 5, 6, 7, 8, 9, or 10 insertions and/or 1, 2, 3, 4, 5, 6, 7, 8, 9, or 10 deletions) in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The invention includes methods involving the use of antibodies, and antigen-binding fragments thereof, that are derived from any of the amino acid sequences disclosed herein, wherein one or more amino acids (e.g. 1, 2, 3, 4, 5, 6, 7, 8, 9, or 10 amino acids) within one or more framework and/or one or more (e.g. 1, 2, 3, 4, 5, 6, 7, 8, 9, 10, 11 or 12 with respect to the tetrameric antibody or 1, 2, 3, 4, 5 or 6 with respect to the HCVR and LCVR of an antibody) CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments that comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. The use of antibodies and antigen-binding fragments obtained in this general manner are encompassed within the invention.

The invention also includes methods involving the use of anti-IL33 antibodies comprising variants of any of the HCVR, LCVR, and/or CDR amino acid sequences disclosed herein having one or more conservative substitutions. For example, the invention includes the use of anti-IL-33 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “surface plasmon resonance” refers to an optical phenomenon that allows for the analysis of real-time interactions by detection of alterations in protein concentrations within a biosensor matrix, for example using the BIAcore™ system (Biacore Life Sciences division of GE Healthcare, Piscataway, N.J.).

The term “K_{D}” refers to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

### Preparation of Human Antibodies

Methods for generating human antibodies in transgenic mice are known in the art. Any such known methods can be used to make human antibodies that specifically bind to human IL-33.

Using VELOCIMMUNE® technology (see, for example, U.S. Pat. No. 6,596,541, Regeneron Pharmaceuticals) or any other known method for generating monoclonal antibodies, high affinity chimeric antibodies to IL-33 are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc., using standard procedures known to those skilled in the art. The mouse constant regions are replaced with a desired human constant region to generate a fully human antibody featured in the invention, for example wild-type or modified IgG1 or IgG4. While the constant region selected may vary according to specific use, high affinity antigen-binding and target specificity characteristics reside in the variable region.

In general, the antibodies that can be used in the methods possess high affinities, as described above, when measured by binding to antigen either immobilized on solid phase or in solution phase. The mouse constant regions are replaced with desired human constant regions to generate the fully human antibodies featured in the invention. While the constant region selected may vary according to specific use, high affinity antigen-binding and target specificity characteristics reside in the variable region.

In one embodiment, human antibody or antigen-binding fragment thereof that specifically binds IL-33 that can be used in the context of the methods featured in the invention comprises the three heavy chain CDRs (HCDR1, HCDR2 and HCDR3) contained within a heavy chain variable region (HCVR) having an amino acid sequence of SEQ ID NO: 2. The antibody or antigen-binding fragment may comprise the three light chain CDRs (LCVR1, LCVR2, LCVR3) contained within a light chain variable region (LCVR) having an amino acid sequence of SEQ ID NO: 10.

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

In certain embodiments, the antibody or antigen-binding fragment thereof comprises the six CDRs (HCDR1, HCDR2, HCDR3, LCDR1, LCDR2 and LCDR3) from the heavy and light chain variable region amino acid sequence pairs (HCVR/LCVR) of SEQ ID NOs: 2 and 10.

In certain embodiments, the antibody or antigen-binding fragment thereof comprises six CDRs (HCDR1/HCDR2/HCDR3/LCDR1/LCDR2/LCDR3) having the amino acid sequences of SEQ ID NOs: 4/6/8/12/14/16.

In certain embodiments, the antibody or antigen-binding fragment thereof comprises HCVR/LCVR amino acid sequence pair of SEQ ID NOs: 2 and 10.

In one embodiment, the antibody is SAR440340, which comprises the HCVR/LCVR amino acid sequence pair of SEQ ID NOs: 2 and 10, and comprises the heavy chain/light chain amino acid sequences pair of SEQ ID NOs: 18 and 20.

### Pharmaceutical Compositions

The invention includes methods that comprise administering an IL-33 antagonist to a patient, wherein the IL-33 antagonist, is contained within a pharmaceutical composition. The invention also includes an IL-33 antagonist for use, wherein the IL-33 antagonist is contained within a pharmaceutical composition. The pharmaceutical compositions featured in the invention are formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J. Pharm. Sci. Technol. 52:238-311.

The dose of antibody administered to a patient may vary depending upon the age and the size of the patient, symptoms, conditions, route of administration, and the like. The dose is typically calculated according to body weight or body surface area. Depending on the severity of the condition, the frequency and the duration of the treatment can be adjusted. Effective dosages and schedules for administering pharmaceutical compositions comprising anti-IL-33 antibodies may be determined empirically. For example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical compositions featured in the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, intra-tracheal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition featured in the invention can be delivered subcutaneously or intravenously with a standard needle and syringe. In addition, with respect to subcutaneous delivery, a pen delivery device (e.g., an autoinjector pen) readily has applications in delivering a pharmaceutical composition featured in the invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™ OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition featured in the invention include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L.P.), and the HUMIRA™ Pen (Abbott Labs, Abbott Park Ill.), to name only a few. Examples of large-volume delivery devices (e.g., large-volume injectors) include, but are not limited to, bolus injectors such as, e.g., BD Libertas West SmartDose, Enable Injections, SteadyMed PatchPump, Sensile SenseTrial, YPsomed YpsoDose, Bespak Lapas, and the like.

For direct administration to the sinuses, the pharmaceutical compositions featured in the invention may be administered using, e.g., a microcatheter (e.g., an endoscope and microcatheter), an aerosolizer, a powder dispenser, a nebulizer or an inhaler. The methods include administration of an IL-33 antagonist to a subject in need thereof, in an aerosolized formulation. For example, aerosolized antibodies to IL-33 may be administered to treat COPD in a patient. Aerosolized antibodies can be prepared as described in, for example, U.S. Pat. No. 8,178,098, incorporated herein by reference in its entirety.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant (e.g., polysorbate 80, HCO-50 (polyoxyethylene (50 mol) adduct of hydrogenated castor oil)), etc. As the oily medium, there are employed, e.g., sesame oil, soybean oil, etc., which may be used in combination with a solubilizing agent such as benzyl benzoate, benzyl alcohol, etc. The injection thus prepared is typically filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

### Dosage

The amount of IL-33 antagonist (e.g., an anti-IL-33 antibody or antigen-binding fragment thereof) administered to a subject according to the methods featured in the invention or for use according to the invention is, generally, a therapeutically effective amount. As used herein, the phrase “therapeutically effective amount” means an amount of IL-33 antagonist that results in one or more of: (a) a reduction in the incidence of COPD exacerbations; (b) an improvement in one or more COPD-associated parameters (as defined elsewhere herein); and/or (c) a detectable improvement in one or more symptoms or indicia of an upper airway inflammatory condition. A “therapeutically effective amount” also includes an amount of IL-33 antagonist that inhibits, prevents, lessens, or delays the progression of COPD in a subject.

In the case of an anti-IL-33 antibody, a therapeutically effective amount can be from about 0.05 mg to about 700 mg, e.g., about 0.05 mg, about 0.1 mg, about 1.0 mg, about 1.5 mg, about 2.0 mg, about 3.0 mg, about 5.0 mg, about 7.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, or about 700 mg of the anti-IL-33 antibody. In certain embodiments, 300 mg of an anti-IL-33 antibody is administered.

The amount of IL-33 antagonist contained within the individual doses may be expressed in terms of milligrams of antibody per kilogram of patient body weight (i.e., mg/kg). For example, the IL-33 antagonist may be administered to a patient at a dose of about 0.0001 to about 10 mg/kg of patient body weight. For example, the IL-33 antagonist can be administered at a dose of 1 mg/kg, 2 mg/kg, 3 mg/kg, or 4 mg/kg.

In certain embodiments, the methods comprise an initial dose of about 200 to about 600 mg of an IL-33 antagonist, e.g., about 300 mg of an IL-33 antagonist.

In certain embodiments, the methods comprise one or more subsequent doses of about 200 to about 400 mg of the IL-33 antagonist, e.g., about 300 mg of an IL-33 antagonist.

In certain embodiments, ICS and LABA are administered for the duration of administration of the IL-33 antagonist. In certain embodiments, ICS and LAMA are administered for the duration of administration of the IL-33 antagonist. In certain embodiments, LAMA and LABA are administered for the duration of administration of the IL-33 antagonist. In certain embodiments, ICS, LAMA and LABA are administered for the duration of administration of the IL-33 antagonist.

In certain embodiments, the initial dose comprises 300 mg of an anti-IL-33 antibody or antigen-binding fragment thereof, and the one or more subsequent doses comprises 300 mg of the antibody or antigen-binding fragment thereof administered every other week.

In other embodiments, the initial dose comprises 300 mg of an anti-IL-33 antibody or antigen-binding fragment thereof, and the one or more subsequent doses comprises 300 mg of the antibody or antigen-binding fragment thereof administered every fourth week.

In other embodiments, the initial dose comprises 300 mg of an anti-IL-33 antibody or antigen-binding fragment thereof, and the one or more subsequent doses comprises 300 mg of the antibody or antigen-binding fragment thereof administered once a week.

In other embodiments, the initial dose comprises 300 mg of an anti-IL-33 antibody or antigen-binding fragment thereof, and the one or more subsequent doses comprises 300 mg of the antibody or antigen-binding fragment thereof administered every third week.

### Combination Therapies

Certain embodiments of the methods featured in the invention comprise administering to the subject one or more additional therapeutic agents in combination with the IL-33 antagonist. Certain embodiments of the invention comprise the IL-33 antagonist for use in combination with additional therapeutic agents. Certain embodiments of the invention comprise a combination of the IL-33 antagonist with additional therapeutic agents for use. As used herein, the expression “in combination with” means that the additional therapeutic agents are administered before, after, or concurrent with the pharmaceutical composition comprising the IL-33 antagonist. In some embodiments, the term “in combination with” includes sequential or concomitant administration of an IL-33 antagonist and an additional therapeutic agent. The invention includes methods to treat COPD or an associated condition or complication or to reduce at least one exacerbation, comprising administration of an IL-33 antagonist, in combination with an additional therapeutic agent for additive or synergistic activity. The invention includes an IL-33 antagonist for use, in combination with an additional therapeutic agent for additive or synergistic activity, to treat COPD or an associated condition or complication or to reduce at least one exacerbation. The invention includes a combination comprising an IL-33 antagonist and an additional therapeutic agent for additive or synergistic activity, for use to treat COPD or an associated condition or complication or to reduce at least one exacerbation.

For example, when administered “before” the pharmaceutical composition comprising an IL-33 antagonist, the additional therapeutic agent may be administered about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes, or about 10 minutes prior to the administration of the pharmaceutical composition comprising the IL-33 antagonist. When administered “after” the pharmaceutical composition comprising an IL-33 antagonist, the additional therapeutic agent may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours, or about 72 hours after the administration of the pharmaceutical composition comprising the IL-33 antagonist. Administration “concurrent” with the pharmaceutical composition comprising an IL-33 antagonist means that the additional therapeutic agent is administered to the subject in a separate dosage form within less than 5 minutes (before, after, or at the same time) of administration of the pharmaceutical composition comprising the IL-33 antagonist or administered to the subject as a single combined dosage formulation comprising both the additional therapeutic agent and the IL-33 antagonist.

The additional therapeutic agent may be, e.g., another IL-33 antagonist, an IL-4R antagonist, an IL-1 antagonist (including, e.g., an IL-1 antagonist as set forth in U.S. Pat. No. 6,927,044), an IL-6 antagonist, an IL-6R antagonist (including, e.g., an anti-IL-6R antibody as set forth in U.S. Pat. No. 7,582,298), a TNF antagonist, an IL-8 antagonist, an IL-9 antagonist, an IL-17 antagonist, an IL-5 antagonist, an IgE antagonist, a CD48 antagonist, a leukotriene inhibitor, an anti-fungal agent, an NSAID, a long-acting muscarinic antagonist (e.g., tiotropium, aclidinium, glycopyrronium bromide or umeclidinium), a long-acting beta2 agonist (e.g., salmeterol or formoterol), an inhaled corticosteroid (e.g., fluticasone or budesonide), a systemic corticosteroid (e.g., oral or intravenous), methylxanthine, nedocromil sodium, cromolyn sodium, or combinations thereof. For example, in certain embodiments, the pharmaceutical composition comprising an IL-33 antagonist is administered with a combination comprising a long-acting beta2 agonist and an inhaled corticosteroid (e.g., fluticasone+salmeterol [e.g., Advair® (GlaxoSmithKline)]; or budesonide+formoterol [e.g., SYMBICORT® (Astra Zeneca)]). In other embodiments, the pharmaceutical composition comprising an IL-33 antagonist is administered with a combination comprising a long-acting muscarinic antagonist and an inhaled corticosteroid (e.g., fluticasone+salmeterol (e.g., Advair® (GaxoSmithKline)); or budesonide+formoterol (e.g., SYMBICORT® (Astra Zeneca))). In still other embodiments, the pharmaceutical composition comprising an IL-33 antagonist is administered with a combination comprising a long-acting muscarinic antagonist, a long-acting beta2 agonist, and an inhaled corticosteroid (e.g., fluticasone+salmeterol (e.g., Advair® (GlaxoSmithKline)); or budesonide+formoterol (e.g., SYMBICORT® (Astra Zeneca))).

### Administration Regimens

According to certain embodiments, multiple doses of an IL-33 antagonist may be administered to a subject (or used) over a defined time course. Such methods comprise sequentially administering to a subject multiple doses of an IL-33 antagonist. As used herein, “sequentially administering” means that each dose of an IL-33 antagonist is administered to the subject at a different point in time, e.g., on different days separated by a predetermined interval (e.g., hours, days, weeks, or months). Included are methods (or uses) that comprise sequentially administering to the patient a single initial dose of an IL-33 antagonist followed by one or more secondary doses of the IL-33 antagonist, and optionally followed by one or more tertiary doses of the IL-33 antagonist.

The invention includes methods (or uses) comprising administering to a subject a pharmaceutical composition comprising an IL-33 antagonist at a dosing frequency of about four times a week, twice a week, once a week (q1w), once every two weeks (bi-weekly or q2w), once every three weeks (tri-weekly or q3w), once every four weeks (monthly or q4w), once every five weeks (q5w), once every six weeks (q6w), once every seven weeks (q7w), once every eight weeks (q8w), once every nine weeks (q9w), once every ten weeks (q10w), once every eleven weeks (q11w), once every twelve weeks (q12w), or less frequently so long as a therapeutic response is achieved. In certain embodiments involving the administration of a pharmaceutical composition comprising an anti-IL-33 antibody once a week dosing of an amount of about 75 mg, 100 mg, 150 mg, 200 mg, or 300 mg, can be employed. In other embodiments involving the administration of a pharmaceutical composition comprising an anti-IL-33 antibody once every two weeks dosing (bi-weekly dosing) of an amount of about 75 mg, 100 mg, 150 mg, 200 mg, or 300 mg, can be employed. In other embodiments involving the administration of a pharmaceutical composition comprising an anti-IL-33 antibody once every three weeks dosing of an amount of about 75 mg, 100 mg, 150 mg, 200 mg, or 300 mg, can be employed. In other embodiments involving the administration of a pharmaceutical composition comprising an anti-IL-33 antibody once every four weeks dosing (monthly dosing) of an amount of about 75 mg, 100 mg, 150 mg, 200 mg, or 300 mg, can be employed. In other embodiments involving the administration of a pharmaceutical composition comprising an anti-IL-33 antibody once every five weeks dosing of an amount of about 75 mg, 100 mg, 150 mg, 200 mg, or 300 mg, can be employed. In other embodiments involving the administration of a pharmaceutical composition comprising an anti-IL-33 antibody once every six weeks dosing of an amount of about 75 mg, 100 mg, 150 mg, 200 mg, or 300 mg, can be employed. In other embodiments involving the administration of a pharmaceutical composition comprising an anti-IL-33 antibody once every eight weeks dosing of an amount of about 75 mg, 100 mg, 150 mg, 200 mg, or 300 mg, can be employed. In other embodiments involving the administration of a pharmaceutical composition comprising an anti-IL-33 antibody once every twelve weeks dosing of an amount of about 75 mg, 100 mg, 150 mg, 200 mg, or 300 mg, can be employed. In one embodiment, the route of administration is subcutaneous.

The term “week” or “weeks” refers to a period of (n×7 days) 3 days, e.g. (n×7 days) ±1 day, or (n×7 days), wherein “n” designates the number of weeks, e.g. 1, 2, 3, 4, 5, 6, 8, 12 or more.

The terms “initial dose,” “secondary doses,” and “tertiary doses,” refer to the temporal sequence of administration of the IL-33 antagonist. Thus, the “initial dose” is the dose that is administered at the beginning of the treatment regimen (also referred to as the “baseline dose”); the “secondary doses” are the doses that are administered after the initial dose; and the “tertiary doses” are the doses that are administered after the secondary doses. The initial, secondary, and tertiary doses may all contain the same amount of IL-33 antagonist, but may differ from one another in terms of frequency of administration. In certain embodiments, however, the amount of IL-33 antagonist contained in the initial, secondary and/or tertiary doses varies from one another (e.g., adjusted up or down as appropriate) during the course of treatment. In certain embodiments, two or more (e.g., 2, 3, 4, or 5 or more) doses are administered at the beginning of the treatment regimen as “initial doses” or “loading doses” followed by subsequent doses that are administered on a less frequent basis (e.g., “maintenance doses”). In one embodiment, the maintenance dose may be lower than the loading or initial dose. For example, one or more loading doses of 600 mg of IL-33 antagonist may be administered followed by maintenance doses of about 75 mg to about 300 mg.

In certain embodiments, the initial dose is about 200 to about 600 mg of the IL-33 antagonist. In one embodiment, the initial dose is 300 mg of the IL-33.

In certain embodiments, the subsequent dose is about 200 to about 300 mg of the IL-33 antagonist. In one embodiment, the subsequent dose is 200 mg of the IL-33 antagonist. In another embodiment, the subsequent dose is 300 mg of the IL-33 antagonist.

In certain embodiments, the initial dose is two times the subsequent dose(s). In certain embodiments, the initial dose is the same amount as the subsequent dose(s).

In some embodiments, the initial dose comprises 300 mg of the antibody or antigen-binding fragment thereof, and the one or more subsequent doses comprises 300 mg of the antibody or antigen-binding fragment thereof administered every other week.

In some embodiments, a subject has moderate-to-severe COPD, and the initial dose comprises 300 mg of the antibody or antigen-binding fragment thereof, and the one or more subsequent doses comprises 300 mg of the antibody or antigen-binding fragment thereof administered every other week.

In some embodiments, the initial dose comprises 300 mg of the antibody or antigen-binding fragment thereof, and the one or more subsequent doses comprises 300 mg of the antibody or antigen-binding fragment thereof administered every fourth week.

In some embodiments, a subject has moderate-to-severe COPD, and the initial dose comprises 300 mg of the antibody or antigen-binding fragment thereof, and the one or more subsequent doses comprises 300 mg of the antibody or antigen-binding fragment thereof administered every fourth week.

In one exemplary embodiment, each secondary and/or tertiary dose is administered 1 to 14 (e.g., 1, 12, 2, 22, 3, 32, 4, 42, 5, 52, 6, 62, 7, 72, 8, 82, 9, 92, 10, 102, 11, 112, 12, 122, 13, 132, 14, 142, or more) weeks after the immediately preceding dose. The phrase “the immediately preceding dose” means, in a sequence of multiple administrations, the dose of IL-33 antagonist that is administered to a patient prior to the administration of the very next dose in the sequence with no intervening doses.

The methods (or uses) may include administering to a patient any number of secondary and/or tertiary doses of an IL-33 antagonist. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple subsequent or secondary doses, each subsequent or secondary dose may be administered at the same frequency as the other subsequent or secondary doses. For example, each subsequent or secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

The invention includes methods comprising sequential administration of an IL-33 antagonist and an additional therapeutic agent, to a patient to treat COPD or an associated condition. The invention also includes an IL-33 antagonist for use to a patient to treat COPD or an associated condition, wherein said IL-33 antagonist is used in sequential administration with an additional therapeutic agent. The invention further includes an IL-33 antagonist for use to a patient to treat COPD or an associated condition, wherein said patient is treated with sequential administration of an IL-33 antagonist and an additional therapeutic agent. In some embodiments, the methods comprise administering one or more doses of an IL-33 antagonist followed by one or more doses (e.g., 2, 3, 4, 5, 6, 7, 8, or more) of an additional therapeutic agent. For example, one or more doses of about 75 mg to about 300 mg of an IL-33 antagonist t may be administered after which one or more doses (e.g., 2, 3, 4, 5, 6, 7, 8, or more) of an additional therapeutic agent (e.g., an inhaled corticosteroid or a beta2-agonist or a muscarinic antagonist or any other therapeutic agent, as described elsewhere herein) may be administered to treat, alleviate, reduce or ameliorate one or more symptoms of COPD. In some embodiments, an IL-33 antagonist is administered at one or more doses (e.g., 2, 3, 4, 5, 6, 7, 8, or more) resulting in an improvement in one or more COPD-associated parameters followed by the administration of a second therapeutic agent to prevent recurrence of at least one symptom of COPD. Alternative embodiments pertain to concomitant administration of an IL-33 antagonist and an additional therapeutic agent. For example, one or more doses (e.g., 2, 3, 4, 5, 6, 7, 8, or more) of an IL-33 antagonist are administered and an additional therapeutic agent is administered at a separate dosage at a similar or different frequency relative to an IL-33 antagonist. In some embodiments, the additional therapeutic agent is administered before, after or concurrently with the IL-33 antagonist.

In certain embodiments, an IL-33 antagonist is administered every other week for 12 weeks, 14 weeks, 16 weeks, 18 weeks, 20 weeks, 22 weeks, 24 weeks, 26 weeks, 28 weeks, 30 weeks, 32 weeks, 34 weeks, 36 weeks, 38 weeks, 40 weeks, 42 weeks, 44 weeks, 46 weeks, 48 weeks or more. In other embodiments, an IL-33 antagonist is administered every four weeks for 12 weeks, 16 weeks, 20 weeks, 24 weeks, 28 weeks, 32 weeks, 36 weeks, 40 weeks, 44 weeks, 48 weeks or more. In specific embodiments, an IL-33 antagonist is administered for at least 24 weeks.

The invention includes methods for treating a subject having moderate-to-severe COPD comprising administering to the subject a loading dose of an antibody or an antigen-binding fragment thereof that specifically binds to IL-33. In certain embodiments, the methods comprise administering to the subject a plurality of maintenance doses of the antibody or the antigen-binding fragment(s) thereof, wherein the plurality of maintenance doses are administered during a treatment phase.

In another aspect, a method for treating a subject having moderate-to-severe COPD comprises administering to the subject an initial dose of about 300 mg of an antibody or an antigen-binding fragment thereof that specifically binds to interleukin-33 (IL-33), and administering to the subject a plurality of subsequent doses of the antibody or the antigen-binding fragment thereof. In another aspect, an antibody or an antigen-binding fragment thereof that specifically binds to interleukin-33 (IL-33) is provided for use to treat a subject having moderate-to-severe COPD, wherein said antibody or antigen-binding fragment is administered to the subject at an initial dose of about 300 mg and then administered to the subject a plurality of times at subsequent doses. In another aspect, it is also provided an antibody or an antigen-binding fragment thereof that specifically binds to interleukin-33 (IL-33) is provided for use to treat a subject having moderate-to-severe COPD, wherein said subject is treated with the antibody or antigen-binding fragment at an initial dose of about 300 mg and then with a plurality of times at subsequent doses. Each subsequent dose is about 300 mg of the antibody or antigen-binding fragment thereof, wherein the plurality of subsequent doses are administered during a treatment phase comprising an induction phase, an oral corticosteroid (OCS) reduction phase, and a maintenance phase, and wherein the antibody or antigen-binding fragment thereof comprises heavy and light chain CDR sequences comprise SEQ ID NOs: 4, 6, 8, 12, 14 and 16.

### Treatment Populations

The methods (or uses) featured in the invention include administering to a subject in need thereof a therapeutic composition comprising an IL-33 antagonist. The expression “a subject in need thereof” means a human or non-human animal that exhibits one or more symptoms or indicia of COPD (e.g., moderate-to-severe COPD), or who has been diagnosed with COPD. For example, “a subject in need thereof” may include, e.g., a subject who, prior to treatment, exhibits (or has exhibited) one or more COPD-associated parameters, such as, e.g., impaired FEV1 (e.g., less than 2.0 L), and/or has experienced one or more exacerbation of COPD events, e.g., acute exacerbation of COPD (AECOPD) events.

As used herein, an “exacerbation of COPD” refers to a period of acute worsening of one or more respiratory symptoms, which may be further characterized by exacerbation rate, time to first exacerbation or having one or more exacerbations. Exacerbations of COPD can include, but are not limited, to increase in dyspnea, increase in wheezing, increase in cough, increase in sputum volume and/or increase in sputum purulence. Acute exacerbations of COPD (AECOPD) may require treatment with systemic corticosteroids (oral, intravenous, or intramuscular), treatment with antibiotics, and/or hospitalization. In various embodiments, the methods may be used to treat mild, moderate, moderate-to-severe, and severe AECOPD events in patients in need thereof.

In some embodiments, a “subject in need thereof” is a subject between the ages of 40 and 75. In some embodiments, the subject is at least 40 years old. In some embodiments, the subject is at least 65 years old. In some embodiments, the subject is 75 years of age or older. In some embodiments, the subject is between 40 and 85 years of age. In some embodiments, the subject is younger than 40 years of age.

In some embodiments, a “subject in need thereof” is a subject who is a current smoker. In some embodiments, the subject is a current smoker who smokes cigarettes. In some embodiments, the subject is a current smoker who has a smoking history of smoking greater than or equal to 10 packs of cigarettes per year. In some embodiments, the subject is a current smoker and has a smoking history of smoking fewer than 10 packs of cigarettes per year. In some embodiments, the subject is a current smoker and has a smoking history of smoking more than 1, 5, 10, 15, 20, 25, 30, 35, 40, 45, 50 or more packs of cigarettes per year. In some embodiments, the subject is a current smoker who has a smoking history of smoking for 6 months, 1 year, 2 years, 3 years, 5 years, 10 years or longer.

In some embodiments, a “subject in need thereof” is a subject who is a former smoker. In some embodiments, the subject is a former smoker who has a history of smoking cigarettes. In some embodiments, the subject is a former smoker who has a smoking history of smoking greater than or equal to 10 packs of cigarettes per year. In some embodiments, the subject is a former smoker who has a smoking history of smoking fewer than 10 packs per year. In some embodiments, the subject is a former smoker who has a smoking history of smoking more than 1, 5, 10, 15, 20, 25, 30, 35, 40, 45, 50 or more packs of cigarettes per year. In some embodiments, the subject is a former smoker who has a smoking history of smoking about 10, 15, 20, 25, 30, 35, 40, 45, 50 or more packs of cigarettes per year. In some embodiments, the subject is a former smoker who has a smoking history of smoking for 6 months, 1 year, 2 years, 3 years, 5 years, 10 years or longer. In some embodiments, the subject is a former smoker who has ceased smoking for at least 1, 2, 3, 4, 5, 6, 7, 8, 9, 10, 11, or 12 months or more. In some embodiments, the subject is a former smoker who has ceased smoking for at least 6 months. In some embodiments, the subject is a former smoker that intends to quit permanently.

In some embodiments, a “subject in need thereof” may be a subject classified as having “mild” COPD based on the GOLD classification system. In other embodiments, a “subject in need thereof” may be a subject classified as having “moderate” COPD based on the GOLD classification system. In another embodiment, a “subject in need thereof” may be a subject classified as having “severe” COPD based on the GOLD classification system. In yet another embodiment, “subject in need thereof” may be a subject classified as having “very severe” COPD based on the GOLD classification system. In another embodiment, a “subject in need thereof” may be a subject classified as having COPD that falls between “moderate” and “severe” based on the GOLD classification system, e.g., a subject having “moderate-to-severe” COPD.

In some embodiments, a “subject in need thereof” may be a subject that has a tested FEV1 value of less than 90%, 85%, 80%, 75%, 70%, 65%, 60%, 55%, 50%, 45%, 40%, 35%, 30%, 20%, 15%, or 10% or less than the predicted FEV1.

A normal IgE level in healthy subjects is less than about 100 kU/L (e.g., as measured using the IMMUNOCAP® assay [Phadia, Inc. Portage, Mich.]). Thus, methods are provided comprising selecting a subject who exhibits an elevated serum IgE level, which is a serum IgE level greater than about 100 kU/L, greater than about 150 kU/L, greater than about 500 kU/L, greater than about 1000 kU/L, greater than about 1500 kU/L, greater than about 2000 kU/L, greater than about 2500 kU/L, greater than about 3000 kU/L, greater than about 3500 kU/L, greater than about 4000 kU/L, greater than about 4500 kU/L, or greater than about 5000 kU/L, and administering to the subject a pharmaceutical composition comprising a therapeutically effective amount of an IL-33 antagonist.

Eotaxin-3 belongs to a group of chemokines released by airway epithelial cells, which is up-regulated by the Th2 cytokines IL-4 and IL-13 (Lilly et al 1999, J. Allergy Clin. Immunol. 104: 786-790). The invention includes methods comprising administering an IL-33 antagonist to treat patients with elevated levels of eotaxin-3, such as more than about 100 pg/ml, more than about 150 pg/ml, more than about 200 pg/ml, more than about 300 pg/ml, or more than about 350 pg/ml. Serum eotaxin-3 levels may be measured, for example, by ELISA.

Fractional exhaled NO (FeNO) is a biomarker of bronchial or airway inflammation. FeNO is produced by airway epithelial cells in response to inflammatory cytokines including IL-4 and IL-13 (Alwing et al 1993, Eur. Respir. J. 6: 1368-1370). FeNO levels in healthy adults range from 2 to 30 parts per billion (ppb). An exemplary assay for measuring FeNO is by using a NIOX instrument by Aerocrine AB, Solna, Sweden. The assessment may be conducted prior to spirometry and following a fast of at least an hour. Included here are methods comprising administering an IL-33 antagonist to patients with elevated levels of exhaled NO (FeNO), such as more than about 30 ppb, more than about 31 ppb, more than about 32 ppb, more than about 33 ppb, more than about 34 ppb, or more than about 35 ppb.

Induced sputum eosinophils and neutrophils are well-established direct markers of airway inflammation (Djukanovic et al 2002, Eur. Respire. J. 37: 1S-2S). Sputum is induced with inhalation of hypertonic saline solution and processed for cell counts according to methods known in the art, for example, the guidelines of European Respiratory Society.

In some embodiments, the subjects are stratified into the following groups: a blood eosinophil count of ≥300 cells/μL (or cells/mm³) or ≥250 cells/μL (or cells/mm³) (high blood eosinophils); a blood eosinophil count of between 299 and 150 cells/μL (or cells/mm³) (moderate blood eosinophils); a blood eosinophil count of <150 cells/μL (or cells/mm³) (low blood eosinophils); or a blood eosinophil count of <300 cells/μL (or cells/mm³), and are administered an IL-33 antagonist at a dose or dosing regimen that is optionally based upon the eosinophil level.

### Methods for Assessing Pharmacodynamic COPD-Associated Parameters

The disclosure also includes methods for assessing one or more pharmacodynamic COPD-associated parameters a subject in need thereof, caused by administration of a pharmaceutical composition comprising an IL-33 antagonist. A reduction in the incidence of an COPD exacerbation (as described above) or an improvement in one or more COPD-associated parameters (as described above) may correlate with an improvement in one or more pharmacodynamic COPD-associated parameters; however, such a correlation is not necessarily observed in all cases.

Examples of “pharmacodynamic COPD-associated parameters” include, for example, the following: (a) biomarker expression levels; (b) serum protein and RNA analysis; (c) induced sputum eosinophils and neutrophil levels; (d) exhaled nitric oxide (FeNO); and (e) blood eosinophil count. An “improvement in a pharmacodynamic COPD-associated parameter” means, for example, a decrease from baseline of one or more biomarkers, such as TARC, eotaxin-3 or IgE, a decrease in sputum eosinophils or neutrophils, FeNO, or blood eosinophil count. As used herein, the term “baseline,” with regard to a pharmacodynamic COPD-associated parameter, means the numerical value of the pharmacodynamic COPD-associated parameter for a patient prior to or at the time of administration of a pharmaceutical composition described herein.

To assess a pharmacodynamic COPD-associated parameter, the parameter is quantified at baseline and at a time point after administration of the pharmaceutical composition. For example, a pharmacodynamic COPD-associated parameter may be measured at day 1, day 2, day 3, day 4, day 5, day 6, day 7, day 8, day 9, day 10, day 11, day 12, day 14, or at week 3, week 4, week 5, week 6, week 7, week 8, week 9, week 10, week 11, week 12, week 13, week 14, week 15, week 16, week 17, week 18, week 19, week 20, week 21, week 22, week 23, week 24, or longer, after the initial treatment with the pharmaceutical composition. The difference between the value of the parameter at a particular time point following initiation of treatment and the value of the parameter at baseline is used to establish whether there has been change, such as an “improvement,” in the pharmacodynamic COPD-associated parameter (e.g., an increase or decrease, as the case may be, depending on the specific parameter being measured).

In certain embodiments, administration of an IL-33 antagonist to a subject having COPD causes a change, such as a decrease or increase, in expression of a particular biomarker.

IL-33-associated biomarkers include, but are not limited to, calcitonin, procalcitonin, calcitonin gene-related peptide (CGRP), resistin-like alpha (RETNA), chemokine (CC motif) ligand 8 (Cc18), serum amyloid A 3 (Saa3), Gm1975 (BC117090), killer cell lectin-like receptor (Kirgl), stefin A1 (Csta), membrane-spanning 4-domain (Ms4a8a), chemokine (CC motif) ligand 11 (Ccl11), and serine (or cysteine) peptides (Serpina3f), and the like.

COPD-associated biomarkers include, but are not limited to, fractional exhaled nitric oxide (FeNO), total IL-33, soluble IL-33 receptor (sST2), calcitonin, PARC, eotaxin-3, total IgE, blood C-reactive protein (CRP), blood IL-6, fibrinogen, and the like.

In certain embodiments, administration of an IL-33 antagonist to a subject having COPD can cause a decrease in in one or more of total serum IgE levels or eotaxin-3 levels. In other embodiments, administration of an IL-33 antagonist to a subject having COPD can cause a decrease in in one or more IL-33-associated biomarkers. The decrease in one or more biomarkers can be detected at week 1, week 2, week 3, week 4, week 5, or longer following administration of the IL-33 antagonist. Biomarker expression can be assayed by methods known in the art. For example, protein levels can be measured by ELISA (enzyme-linked immunosorbent assay). RNA levels can be measured, for example, by reverse transcription coupled to polymerase chain reaction (RT-PCR).

Biomarker expression, as discussed above, can be assayed by detection of protein or RNA in serum. The serum samples can also be used to monitor additional protein or RNA biomarkers related to response to treatment with an IL-33 antagonist. In some embodiments, RNA samples are used to determine RNA levels (non-genetic analysis), e.g., RNA levels of biomarkers, and in other embodiments, RNA samples are used for transcriptome sequencing (e.g., genetic analysis).

### EXAMPLES

The following examples are put forth so as to provide those of ordinary skill in the art with a complete disclosure and description of how to make and use the methods and compositions featured in the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

The exemplary IL-33 antagonist used in the following examples is the human anti-IL-33 antibody named SAR440340, which is also referred to as REGN3500 or by its international nonproprietary name (INN), itepekimab.

### Example 1. A Randomized, Double-Blind, Placebo-Controlled, Parallel-Group, Proof-of-Concept (PoC) Study to Assess the Efficacy, Safety and Tolerability of SAR440340, in Patients with Moderate-to-Severe Chronic Obstructive Pulmonary Disease (COPD)

### A. Study Objectives, Endpoints, and Overview

Chronic obstructive pulmonary disease (COPD) is a highly prevalent disease worldwide, associated with significant economic burden, and for which available standard-of-care therapy shows insufficient treatment effect on symptoms, lung function, exacerbations and long term evolution of the disease. Interleukin-33 (IL-33) is a pro-inflammatory cytokine that initiates and amplifies innate and adaptive inflammatory cascades, in response to epithelial cell stress or damage due to exposure to airborne allergens, viruses, cigarette smoke, and air pollutants.

The primary objective of the study was to investigate effects of SAR440340 (anti-IL-33 mAb) compared with placebo, on the annualized rate of moderate-to-severe acute exacerbations of COPD (AECOPD).

The secondary objectives of the study were: to investigate effects of SAR440340 compared with placebo, on improving respiratory function, as assessed by pre-bronchodilator FEV1; to evaluate effects of SAR440340 compared with placebo, on post-bronchodilator FEV1; to evaluate effects of SAR440340 compared with placebo, on duration from baseline to first moderate or severe AECOPD event; and to evaluate effects of SAR440340 compared with placebo, on safety and tolerability.

The exploratory objectives of the study were: to evaluate the effects of SAR440340 compared with placebo, on patient reported symptoms and quality of life as documented by e-Diary and utilizing Exacerbations of Chronic Obstructive Pulmonary Disease Tool (EXACT), St. George's Respiratory Questionnaire (SGRQ) and Euroqol-5Dimension (EQ 5D) questionnaire: in all patients treated with SAR440340/placebo and in subpopulations with high blood eosinophil level (≥250/mm³) and low blood eosinophil level (<250/mm³); to evaluate the pharmacokinetic (PK) profile of SAR440340 in serum; to evaluate the effects of SAR440340 Antidrug antibodies (ADA); to evaluate the effects of SAR440340 compared with placebo, on FEV1, AECOPD, and other select endpoints: in subpopulations with high blood eosinophil level (≥250/mm³) and low blood eosinophil level (<250/mm³) and in subpopulations according to use/no use of ICS with bronchodilators as background therapy, fibrinogen levels, and smoking status; to evaluate the effects of pharmacogenomics on SAR440340; to evaluate the effects of SAR440340 compared with placebo on other respiratory assessments (expanded AECOPD endpoint); to evaluate the clinical symptoms of COPD in patients treated with SAR440340 versus placebo in all patients treated with SAR440340/placebo: in subpopulations with high blood eosinophil level (≥250/mm³) and low blood eosinophil level (<250/mm³) and in subpopulations according to use/no use of ICS with bronchodilators as background therapy, fibrinogen levels, and smoking status; to evaluate the pharmacodynamics effects of SAR440340; to evaluate the effects of SAR440340 compared to placebo on parameters of sleep, activity, and at-home spirometry; and to compare the utility of at-home spirometry against in-clinic spirometry.

The primary endpoint of the study was annualized rate of moderate-to-severe (AECOPD) over the treatment period. Moderate exacerbations were recorded by the Investigator and defined as AECOPD that require either systemic corticosteroids (such as intramuscular, intravenous or oral) and/or antibiotics. Severe exacerbations were recorded by the Investigator and defined as AECOPD requiring hospitalization, emergency medical care visit or resulting in death.

A secondary endpoint was average change from baseline to week 16-24 in FEV1 (pre-bronchodilator). Model-based averages across weeks 16, 20 and 24 were be compared between the treatment groups.

Another secondary endpoint was change from baseline to week 24 in FEV1 (post-bronchodilator). Post-bronchodilator means 30 minutes after either 400 mcg of salbutamol/albuterol (4 puffs of 100 mcg each) or 80 mcg of ipratropium bromide (4 puffs of 20 mcg each).

Yet another secondary endpoint was time to first moderate or severe AECOPD.

Still other secondary endpoints were treatment-emergent adverse events (TEAE) and serious adverse events (SAE).

Tertiary endpoints included change from baseline in EXACT, SGRQ, or EQ-5D scores at week 24.

Other tertiary endpoints included: serum functional SAR440340 concentrations; antidrug antibodies (ADA) against SAR440340; change from baseline to Week 24 in FEV1 (pre-bronchodilator and post-bronchodilator); and rate of moderate-to-severe AECOPD.

Still other tertiary endpoints included: future Assessment of DNA or RNA samples for the pharmacogenomics sub study to identify genomic associations with clinical or biomarker response, and other clinical outcome measures and possible AEs; change from baseline to Week 16-24 in FVC (% predicted and absolute values in mL); time to the first moderate and severe exacerbations or time to study drug discontinuation (after Week 4) due to lack of efficacy, based on the investigator's judgement (expanded AECOPD endpoint); and time to first Clinically Important Deterioration (CID) as defined by decrease of >100 mL from baseline in trough FEV1 and/or deterioration in SGRQ by 4 units and/or moderate-to-severe AECOPD up to Week 24 (and over the 52 week variable treatment period.

Other tertiary endpoints related to pharmacodynamics including blood eosinophil and neutrophil counts; levels of biomarkers of the interleukin (IL)-33 and/or Type 2 inflammation pathway including Total IL-33, sST2 levels, calcitonin levels, PARC levels, eotaxin-3 levels, total IgE levels, and Fibrinogen levels; induced sputum for RNA expression (optional for patients at a subset of sites); optionally messenger ribonucleic acid sequencing or whole transcriptome analysis; and optionally DNA/RNA sample are collected for pharmacogenomic effects.

Other tertiary endpoints related to actigraphy (sleep and activity) and home spirometry including change from average measurement over baseline (2 weeks prior to randomization) to average measurements over weeks 10-12 (2 weeks prior to visit 8) and Weeks 22-24 (2 weeks prior to visit 14) of sleep and activity parameters including sleep (total sleep time, wake after sleep onset, overnight activity counts), activity (daytime activity counts, percent of time spent in sedentary activity, percent of time spent in moderate to vigorous physical activity), and spirometry (FEV1). FEV1 measurements were obtained both from spirometry performed at home and at the clinic.

### B. Study Design

This study was a multinational, randomized, double-blind, placebo controlled, parallel group (2 groups), proof of concept (PoC) study that was designed to assess the efficacy, safety, and tolerability of SAR440340 in patients with moderate-to-severe COPD on an established long-acting 2 adrenergic agonist (LABA), long-acting muscarinic antagonist (LAMA), and/or ICS background therapy (double or triple therapy). Patients were to be treated with SAR440340 or placebo for a minimum of 24 weeks and up to a maximum of 52 weeks*, and a 20-week safety follow-up period. Approximately 343 patients were randomized into 2 treatment groups of 171 or 172 patients per group. *The study employed a variable treatment duration from 24 to 52 weeks to maximize data for the primary endpoint (annualized rate of exacerbation) in a time-efficient manner. Patients enrolled in the trial remained in the treatment period for up to a maximum of 52 weeks or until the last patient randomized completed a minimum treatment period of 24 weeks.

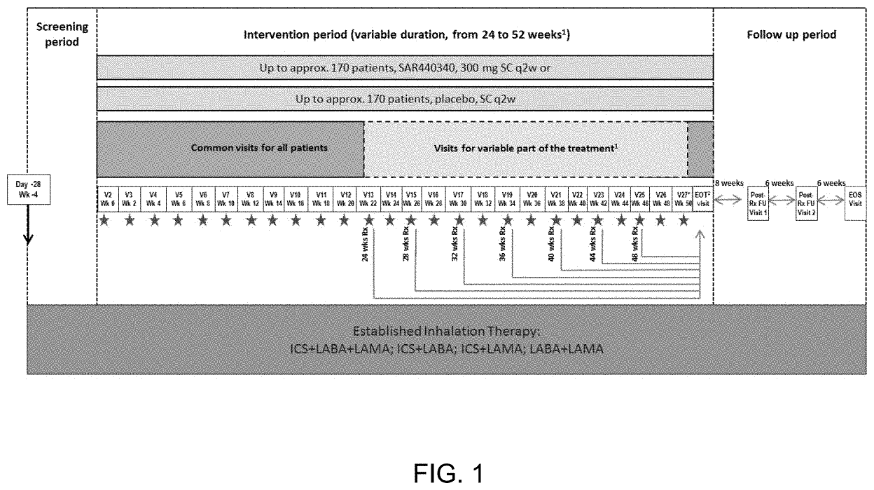

As shown in FIG. 1, the clinical trial consisted of three periods. First, a screening period (10 days to 4 weeks) to determine whether patients met entry criteria wherein patients are on standard of care background therapy, for 3 months prior to visit 2/randomization and at a stable dose for at least 1 month prior to the screening visit 1, including either double therapy (LABA+LAMA or ICS+LABA or ICS+LAMA) or triple therapy (ICS+LABA+LAMA). Second, the randomized treatment period wherein patients who satisfy the inclusion and exclusion criteria are randomized to be included in either the treatment group that receives SAR440340 (300 mg) administered as 2SC injections every 2 weeks (q2w) for 24 to 52 weeks or matching placebo for SAR440340 administered as 2SC injections q2w for 24 to 52 weeks. Third, a post-treatment period that includes 20 weeks of observational follow-up.

The Schedule of Activities (SoA) for patients who complete the planned treatment is described in Table 1.

TABLE 1
Schedule of Activities (SoA) for Patients who Complete the Planned Treatment.

| | S | | | | | | | | | | | | | | | |
|---|---|---|---|---|---|---|---|---|---|---|---|---|---|---|---|---|
| | 1 | | | | | | | | | | | | | | | |
| | W −4 | R/B | | | | | | | | | | | | | | |
| VISIT | to | 2^{a, b} | 3 | 4 | 5 | 6 | 7 | 8 | 9 | 10 | 11 | 12 | 13 | 14 | 15 | 16 |
| WEEK | W −1 | 0 | 2 | 4 | 6 | 8 | 10 | 12 | 14 | 16 | 18 | 20 | 22 | 24 | 26 | 28 |
| Informed | X | | | | | | | | | | | | | | | |
| consent | | | | | | | | | | | | | | | | |
| Patient | X | | | | | | | | | | | | | | | |
| demography | | | | | | | | | | | | | | | | |
| Previous | X | | | | | | | | | | | | | | | |
| medical | | | | | | | | | | | | | | | | |
| and | | | | | | | | | | | | | | | | |
| surgical | | | | | | | | | | | | | | | | |
| history | | | | | | | | | | | | | | | | |
| Chest X- | X | | | | | | | | | | | | | | | |
| ray^{e} | | | | | | | | | | | | | | | | |
| Inclusion/ | X | X | | | | | | | | | | | | | | |
| exclusion | | | | | | | | | | | | | | | | |
| COPD | X | X | | | | | | | | | | | | | | |
| Assessment | | | | | | | | | | | | | | | | |
| Test | | | | | | | | | | | | | | | | |
| (CAT)^{f} | | | | | | | | | | | | | | | | |
| Smoking | X | X | X | X | X | X | X | X | | X | | X | | X | | X |
| status | | | | | | | | | | | | | | | | |
| Prior & | X | X | X | X | X | X | X | X | X | X | X | X | X | X | X | X |
| concomitant | | | | | | | | | | | | | | | | |
| medications | | | | | | | | | | | | | | | | |
| Study | | | | | | | | | | | | | | | | |
| treatment | | | | | | | | | | | | | | | | |
| administration | | | | | | | | | | | | | | | | |
| Call | X | X | X | X | X | X | X | X | X | X | X | X | X | X | X | X |
| IVRS/IWRS | | | | | | | | | | | | | | | | |
| Randomization | | X | | | | | | | | | | | | | | |
| IMP | | X | X | X | X | X | X | X | X | X | X | X | X | X | X | X |
| administration^{g} | | | | | | | | | | | | | | | | |
| Dispense | X | X | X | X | X | X | X | X | X | X | X | X | X | X | X | X |
| or upload | | | | | | | | | | | | | | | | |
| electronic | | | | | | | | | | | | | | | | |
| diary^{h} | | | | | | | | | | | | | | | | |
| Safety | | | | | | | | | | | | | | | | |
| Physical | X | X | | | | | | X | | | | | | X | | |
| examinationⁱ | | | | | | | | | | | | | | | | |
| Vital signs^{j} | X | X | X | X | X | X | X | X | X | X | X | X | X | X | X | X |
| Electrocardiogram | X | X | | | | | | X | | | | | | X | | |
| (12 lead)^{k} | | | | | | | | | | | | | | | | |
| Hematology, | X | X | | | | X | | | | X | | | | X | | |
| biochemistry, | | | | | | | | | | | | | | | | |
| urinalysis | | | | | | | | | | | | | | | | |
| including | | | | | | | | | | | | | | | | |
| cotinine^{l} | | | | | | | | | | | | | | | | |
| Hepatitis | X | | | | | | | | | | | | | | | |
| and HIV | | | | | | | | | | | | | | | | |
| Serology | | | | | | | | | | | | | | | | |
| tests^{m} | | | | | | | | | | | | | | | | |
| Quantiferon | X | | | | | | | | | | | | | | | |
| Gold | | | | | | | | | | | | | | | | |
| Pregnancy | X | | | | | | | | | | | | | | | |
| (β-HCG | | | | | | | | | | | | | | | | |
| blood) | | | | | | | | | | | | | | | | |
| testⁿ | | | | | | | | | | | | | | | | |
| Urine | | X | | X | | X | | X | | X | | X | | X | | X |
| pregnancy | | | | | | | | | | | | | | | | |
| testⁿ | | | | | | | | | | | | | | | | |
| Adverse | X | X | X | X | X | X | X | X | X | X | X | X | X | X | X | X |
| event | | | | | | | | | | | | | | | | |
| reporting, | | | | | | | | | | | | | | | | |
| including | | | | | | | | | | | | | | | | |
| SAEs | | | | | | | | | | | | | | | | |
| Pharmacokinetics | | | | | | | | | | | | | | | | |
| Serum | | X | X | X | | X | | X | | X | | | | X | | |
| samples for | | | | | | | | | | | | | | | | |
| SAR440340 | | | | | | | | | | | | | | | | |
| concentration^{o} | | | | | | | | | | | | | | | | |
| Anti- | | X | | | | | | X | | | | | | X | | |
| SAR440340 | | | | | | | | | | | | | | | | |
| antibody^{p} | | | | | | | | | | | | | | | | |
| Biomarkers | | | | | | | | | | | | | | | | |
| Blood | X | X | | X^{q} | | X | | X^{q} | | | | | | X | | |
| eosinophils | | | | | | | | | | | | | | | | |
| and | | | | | | | | | | | | | | | | |
| neutrophils | | | | | | | | | | | | | | | | |
| Total IL33 | X | X | | X | | X | | X | | | | | | X | | |
| and sST2^{o} | | | | | | | | | | | | | | | | |
| Calcitonin | X | X | | X | | X | | X | | | | | | X | | |
| PARC | X | X | | X | | | | X | | | | | | X | | |
| Eotaxin-3 | X | X | | X | | | | X | | | | | | X | | |
| Total IgE | X | X | | X | | | | X | | | | | | X | | |
| Fibrinogen | X | X | | | | | | | | | | | | X | | |
| FeNO pre- | | X | | X | | X | | X | | | | | | X | | |
| bronchodilator | | | | | | | | | | | | | | | | |
| (optional)^{r} | | | | | | | | | | | | | | | | |
| FeNO | | X | | X | | X | | X | | | | | | X | | |
| post- | | | | | | | | | | | | | | | | |
| bronchodilator | | | | | | | | | | | | | | | | |
| (optional)^{r} | | | | | | | | | | | | | | | | |
| Induced | | X | | | | | | | | | | | | X | | |
| sputum | | | | | | | | | | | | | | | | |
| (optional)^{s} | | | | | | | | | | | | | | | | |
| Blood | X | X | | X | | | | | | | | | | X | | |
| samples | | | | | | | | | | | | | | | | |
| for RNA | | | | | | | | | | | | | | | | |
| sample | | | | | | | | | | | | | | | | |
| (optional) | | | | | | | | | | | | | | | | |
| Blood | X | X | | X | | | | X | | | | | | X | | |
| sample | | | | | | | | | | | | | | | | |
| archival | | | | | | | | | | | | | | | | |
| for | | | | | | | | | | | | | | | | |
| exploratory | | | | | | | | | | | | | | | | |
| research | | | | | | | | | | | | | | | | |
| (optional)^{t} | | | | | | | | | | | | | | | | |
| DNA | | X | | | | | | | | | | | | | | |
| Pharmacogenomics | | | | | | | | | | | | | | | | |
| analysis | | | | | | | | | | | | | | | | |
| (optional) | | | | | | | | | | | | | | | | |
| Efficacy | | | | | | | | | | | | | | | | |
| COPD | | X | X | X | X | X | X | X | X | X | X | X | X | X | X | X |
| exacerbation | | | | | | | | | | | | | | | | |
| reporting | | | | | | | | | | | | | | | | |
| by the | | | | | | | | | | | | | | | | |
| Investigator | | | | | | | | | | | | | | | | |
| Spirometry | X | X | X | X | | X | | X | | X | | X | | X | | X |
| (pre-BD)^{u, v} | | | | | | | | | | | | | | | | |
| Spirometry | X | X | X | X | | X | | X | | | | | | X | | |
| (post-BD)^{u, v} | | | | | | | | | | | | | | | | |
| EXACT^{w} | Every day (diary) from screening to week 52 | | | | | | | | | | | | | | | |
| SGRQ^{w} | | X | | X | | X | | X | | | | | | X | | |
| EQ-5D-5L^{w} | | X | | | | | | | | | | | | X | | |

| | | | |
|---|---|---|---|
| Actigraphy | Con- | Con- | Con- |
| Optional | tinuos | tinuos | tinuos |
| assessments | | | |
| (US/Canada | | | |
| only) | | | |
| At-home | BID | BID | BID |
| spirometry | | | |
| (FEV1) | | | |
| Optional | | | |
| assessments | | | |
| (US/Canada | | | |
| only) | | | |

| VISIT | 17 | 18 | 19 | 20 | 21 | 22 | 23 | 24 | 25 | 26 | 27 | EOT^{c} | F1 | F2^{d} | EOS |
|---|---|---|---|---|---|---|---|---|---|---|---|---|---|---|---|
| WEEK | 30 | 32 | 34 | 36 | 38 | 40 | 42 | 44 | 46 | 48 | 50 | 52 | 60 | 66 | 72 |
| Informed | | | | | | | | | | | | | | | |
| consent | | | | | | | | | | | | | | | |
| Patient | | | | | | | | | | | | | | | |
| demography | | | | | | | | | | | | | | | |
| Previous | | | | | | | | | | | | | | | |
| medical | | | | | | | | | | | | | | | |
| and | | | | | | | | | | | | | | | |
| surgical | | | | | | | | | | | | | | | |
| history | | | | | | | | | | | | | | | |
| Chest X- | | | | | | | | | | | | | | | |
| ray^{e} | | | | | | | | | | | | | | | |
| Inclusion/ | | | | | | | | | | | | | | | |
| exclusion | | | | | | | | | | | | | | | |
| COPD | | | | | | | | | | | | | | | |
| Assessment | | | | | | | | | | | | | | | |
| Test | | | | | | | | | | | | | | | |
| (CAT)^{f} | | | | | | | | | | | | | | | |
| Smoking | | X | | X | | X | | X | | X | X | X | X | X | X |
| status | | | | | | | | | | | | | | | |
| Prior & | X | X | X | X | X | X | X | X | X | X | X | X | X | X | X |
| concomitant | | | | | | | | | | | | | | | |
| medications | | | | | | | | | | | | | | | |
| Study | | | | | | | | | | | | | | | |
| treatment | | | | | | | | | | | | | | | |
| administration | | | | | | | | | | | | | | | |
| Call | X | X | X | X | X | X | X | X | X | X | X | X | | | X |
| IVRS/IWRS | | | | | | | | | | | | | | | |
| Randomization | | | | | | | | | | | | | | | |
| IMP | X | X | X | X | X | X | X | X | X | X | X | | | | |
| administration^{g} | | | | | | | | | | | | | | | |
| Dispense | X | X | X | X | X | X | X | X | X | X | X | X | X | | X |
| or upload | | | | | | | | | | | | | | | |
| electronic | | | | | | | | | | | | | | | |
| diary^{h} | | | | | | | | | | | | | | | |
| Safety | | | | | | | | | | | | | | | |
| Physical | | | | X | | | | | | | | X | | | X |
| examinationⁱ | | | | | | | | | | | | | | | |
| Vital signs^{j} | X | X | X | X | X | X | X | X | X | X | X | X | X | | X |
| Electrocardiogram | | | | X | | | | | | | | X | | | X |
| (12 lead)^{k} | | | | | | | | | | | | | | | |
| Hematology, | | | | X | | | | | | | | X | | | X |
| biochemistry, | | | | | | | | | | | | | | | |
| urinalysis | | | | | | | | | | | | | | | |
| including | | | | | | | | | | | | | | | |
| cotinine^{l} | | | | | | | | | | | | | | | |
| Hepatitis | | | | | | | | | | | | | | | |
| and HIV | | | | | | | | | | | | | | | |
| Serology | | | | | | | | | | | | | | | |
| tests^{m} | | | | | | | | | | | | | | | |
| Quantiferon | | | | | | | | | | | | | | | |
| Gold | | | | | | | | | | | | | | | |
| Pregnancy | | | | | | | | | | | | | | | |
| (β-HCG | | | | | | | | | | | | | | | |
| blood) | | | | | | | | | | | | | | | |
| testⁿ | | | | | | | | | | | | | | | |
| Urine | | X | | X | | X | | X | | X | | X | | | X |
| pregnancy | | | | | | | | | | | | | | | |
| testⁿ | | | | | | | | | | | | | | | |
| Adverse | X | X | X | X | X | X | X | X | X | X | X | X | X | X | X |
| event | | | | | | | | | | | | | | | |
| reporting, | | | | | | | | | | | | | | | |
| including | | | | | | | | | | | | | | | |
| SAEs | | | | | | | | | | | | | | | |
| Pharmacokinetics | | | | | | | | | | | | | | | |
| Serum | | | | X | | | | | | | | X | X | | X |
| samples for | | | | | | | | | | | | | | | |
| SAR440340 | | | | | | | | | | | | | | | |
| concentration^{o} | | | | | | | | | | | | | | | |
| Anti- | | | | | | | | | | | | X | | | X |
| SAR440340 | | | | | | | | | | | | | | | |
| antibody^{p} | | | | | | | | | | | | | | | |
| Biomarkers | | | | | | | | | | | | | | | |
| Blood | | | | X | | | | | | | | X | X^{q} | | X |
| eosinophils | | | | | | | | | | | | | | | |
| and | | | | | | | | | | | | | | | |
| neutrophils | | | | | | | | | | | | | | | |
| Total IL33 | | | | | | | | | | | | X | X | | X |
| and sST2^{o} | | | | | | | | | | | | | | | |
| Calcitonin | | | | | | | | | | | | X | X | | X |
| PARC | | | | X | | | | | | | | X | | | |
| Eotaxin-3 | | | | X | | | | | | | | X | | | |
| Total IgE | | | | | | | | | | | | X | | | |
| Fibrinogen | | | | | | | | | | | | X | | | |
| FeNO pre- | | | | X | | | | | | | | | | | |
| bronchodilator | | | | | | | | | | | | | | | |
| (optional)^{r} | | | | | | | | | | | | | | | |
| FeNO | | | | X | | | | | | | | | | | |
| post- | | | | | | | | | | | | | | | |
| bronchodilator | | | | | | | | | | | | | | | |
| (optional)^{r} | | | | | | | | | | | | | | | |
| Induced | | | | | | | | | | | | X | | | |
| sputum | | | | | | | | | | | | | | | |
| (optional)^{s} | | | | | | | | | | | | | | | |
| Blood | | | | | | | | | | | | X | | | |
| samples | | | | | | | | | | | | | | | |
| for RNA | | | | | | | | | | | | | | | |
| sample | | | | | | | | | | | | | | | |
| (optional) | | | | | | | | | | | | | | | |
| Blood | | | | X | | | | | | | | X | | | |
| sample | | | | | | | | | | | | | | | |
| archival | | | | | | | | | | | | | | | |
| for | | | | | | | | | | | | | | | |
| exploratory | | | | | | | | | | | | | | | |
| research | | | | | | | | | | | | | | | |
| (optional)^{t} | | | | | | | | | | | | | | | |
| DNA | | | | | | | | | | | | | | | |
| Pharmacogenomics | | | | | | | | | | | | | | | |
| analysis | | | | | | | | | | | | | | | |
| (optional) | | | | | | | | | | | | | | | |
| Efficacy | | | | | | | | | | | | | | | |
| COPD | X | X | X | X | X | X | X | X | X | X | X | X | X | X | X |
| exacerbation | | | | | | | | | | | | | | | |
| reporting | | | | | | | | | | | | | | | |
| by the | | | | | | | | | | | | | | | |
| Investigator | | | | | | | | | | | | | | | |
| Spirometry | | X | | X | | X | | X | | X | X | X | X | | X |
| (pre-BD)^{u, v} | | | | | | | | | | | | | | | |
| Spirometry | | | | X | | | | | | | | X | X | | X |
| (post-BD)^{u, v} | | | | | | | | | | | | | | | |
| EXACT^{w} | Every day (diary) from screening to week 52 | | | | | | | | | | | | | | |
| SGRQ^{w} | | | | X | | | | | | | | X | X | | X |
| EQ-5D-5L^{w} | | | | | | | | | | | | X | X | | X |
| Actigraphy | | | | | | | | | | | | | | | |
| Optional | | | | | | | | | | | | | | | |
| assessments | | | | | | | | | | | | | | | |
| (US/Canada | | | | | | | | | | | | | | | |
| only) | | | | | | | | | | | | | | | |
| At-home | | | | | | | | | | | | | | | |
| spirometry | | | | | | | | | | | | | | | |
| (FEV1) | | | | | | | | | | | | | | | |
| Optional | | | | | | | | | | | | | | | |
| assessments | | | | | | | | | | | | | | | |
| (US/Canada | | | | | | | | | | | | | | | |
| only) | | | | | | | | | | | | | | | |

S = Screening; R/B = Randomization/Baseline; F = Follow-up
β-hCG = Human chorionic gonadotropin-beta; BID = twice daily assessments; CAT = COPD Assessment Test; continuous = subsequent visits during the treatment period; COPD = chronic obstructive pulmonary disease; cont. = continuous; DNA = Deoxyribonucleic acid; EDTA = Ethylenediaminetetraacetic acid; EOS = End of Study; EOT = End of treatment; EQ-5D = Euro Quality of Life-5 Dimension questionnaire; EXACT = Exacerbations of COPD tool (EXACT); HIV = human immunodeficiency virus; IgE = Immunoglobulin E; IMP = Investigational Medical Product; IVRS = Interactive Voice Response System; IWRS = Interactive Web Response System; LABA = Long-acting β2 adrenergic agonist; LAMA = Long-acting muscarinic antagonist; PARC = Pulmonary and activation-regulated chemokine; PK = Pharmacokinetic; RNA = Ribonucleic acid; SABA = Short-acting beta-agonists; SAEs = Serious adverse events; SC = subcutaneous; SGRQ = St. George's Respiratory Questionnaire.
^{a}Randomization/baseline Visit is defined as Day 1. The visit schedule should be adhered to within ±3 days for the screening period and randomized IMP treatment period, and ±5 days for the 2 visits during the post IMP treatment period.
^{b}All assessments at Visit 2 (Day 1) are to be conducted pre-IMP dose with the exception of the assessment of local tolerability of SC injections.
^{c}End-of-treatment visit:
^{d}Can be performed with a phone call.
^{e}Chest X-ray to be performed unless a <6 month old chest x-ray/chest CT/chest MRI is available. In case chest-X-ray is not feasible due to local regulations, magnetic resonance imaging (MRI) will be performed.
^{f}The COPD Assessment Test (CAT) is to be registered through the patient's electronic diary.
^{g}IMP (SAR440340 or placebo) to be administered every 2 weeks at the site. Last dose will be given 2 weeks prior to planned EOT visit e.g., for patients with 52-week treatment period the last dose will be taken at Week 50, or earlier, as directed by the sponsor. Patients should be monitored by site personnel for at least 30 minutes after administration of all IMP injections. Monitoring period may be extended as per country specific requirements.
^{h}Electronic diary is used for recording of patient's answers to the EXACT, SGRQ and EQ-5D-5L questionnaires, CAT assessment as well as for recording reliever medication. This device is dispensed at Screening Visit 1 (including instructions for use) and recorded information is downloaded from this device on the other indicated days. At EOS visit the electronic diary is downloaded and returned to the site.
ⁱComplete physical examinations will include skin, nasal cavities, eyes, ears, respiratory, cardiovascular, gastrointestinal, neurological, lymphatic, and musculoskeletal systems.
^{j}Vital signs, including systolic and diastolic blood pressure (mmHg), pulse rate (beats per minute), body temperature (° C.), and respiratory rate will be measured at screening, baseline and every subsequent on-site visit. Height (cm) will be measured at screening (Visit 1) only. Body weight (kg) will be measured at screening (Visit 1) and at EOT/EOS visits.
^{k}ECG to be centrally collected & read.
^{l}Hematology will include hemoglobin, hematocrit, platelet count, total white blood cell count with 5-part differential count, and total red blood cell count. Serum chemistry will include creatinine, blood urea nitrogen, glucose, lactate dehydrogenase, uric acid, total cholesterol, total protein, albumin, total bilirubin, alanine aminotransferase, aspartate aminotransferase, alkaline phosphatase, electrolytes (sodium, potassium, chloride), bicarbonate, and creatine phosphokinase. Urinalysis will include specific gravity, pH, glucose, ketones, blood, protein, nitrate, leukocyte esterase, urobilinogen and bilirubin. If any parameter on the dipstick is abnormal, a urine sample should be sent to the central laboratory for quantitative measurement. If positive for protein and/or red blood cells, microscopic analysis will be performed by the central laboratory. Cotinine will be tested using the urine sample collected.
^{m}Clinical laboratory testing at Screening Visit 1 will include hepatitis screen covering hepatitis B surface antigen (HBs Ag), hepatitis B surface antibody (HBs Ab), hepatitis B core antibody (HBc Ab), hepatitis C virus antibodies (HCV Ab), Human Immunodeficiency Virus (HIV) screen (Anti-HIV-1 and HIV-2 antibodies) and anti-nuclear antibody (ANA). In case of results showing HBs Ag (negative), and HBc Ab (positive), an HBV DNA testing may be performed prior to randomization to rule out a false positivity if the Investigator believes the patient is a false positive, or to clarify the serological status if the Investigator finds it unclear to interpret in absence of known HBV infection. In case of results showing HCV Ab (positive), an HCV RNA testing may be performed to rule out a false positivity, if the Investigator believes the patient is a false positive.
Note:
Anti-ds DNA antibody will be tested if ANA is positive (≥1:160 titer).
ⁿOnly for women of childbearing potential: serum pregnancy test at Screening/V1 and urine pregnancy tests at every 4 weeks from Randomization through EOT and at EOS, A negative result must be obtained at V1 and at V2 prior to randomization. In case of positive urinary test the study treatment will be withheld and a serum pregnancy test to confirm the pregnancy should be performed as soon as possible. Pregnancy will lead to definitive treatment discontinuation in all cases.
^{o}Refer to central lab manual for collection details.
^{p}If ADA assessment at week 12 is positive, additional measurements may be performed from PK samples collected at Week 4.
^{q}The complete hematology panel will be performed.
^{r}FeNO: measurement at sites only with access to FeNO equipment.
^{s}Optional sputum sample at V2 should be collected post-randomization and prior to dosing with IMP, as long as patient consents to the optional collection. Only available at select sites in some countries.
^{t}Archived samples may be used for research purposes related to COPD or other respiratory diseases such as asthma or inflammatory diseases (e.g., exploratory biomarkers of disease or drug effect), pathway biology, additional drug safety assessments or development and validation of bioassay methods beyond those defined in the present protocol.
^{u}Spirometry will be done locally according to European Respiratory Society (ERS)/American Thoracic Society (ATS) 2005 guidance but measured by a central laboratory. Spirometry will be performed during a trough period of bronchodilators according to their action duration, for example, withholding the last dose of salbutamol/albuterol or levosalbutamol/levalbuterol for at least 6 hours, withholding the last dose of ipratropium for at least 8 hours, withholding the last dose of LABA for at least 12 hours (ultra-long acting LABA like vilanterol should be withheld for at least 24 hours) and withholding the last dose of LAMA for at least 24 hours. This will be verified before performing the measurements.
Note:
When both pre and post-bronchodilator spirometry is assessed, the post-bronchodilator spirometry should be performed consistent with the mechanism of action of reliever (i.e., 30 minutes for albuterol or other SABA).
^{v}Following randomization and during the treatment period - if spirometry is not performed at the scheduled visit it should be performed at the following visit during the treatment period.
^{w}The EXACT, SGRQ and EQ-5D-5L are to be completed in the patient's electronic diary.

### C. Selection of Patients

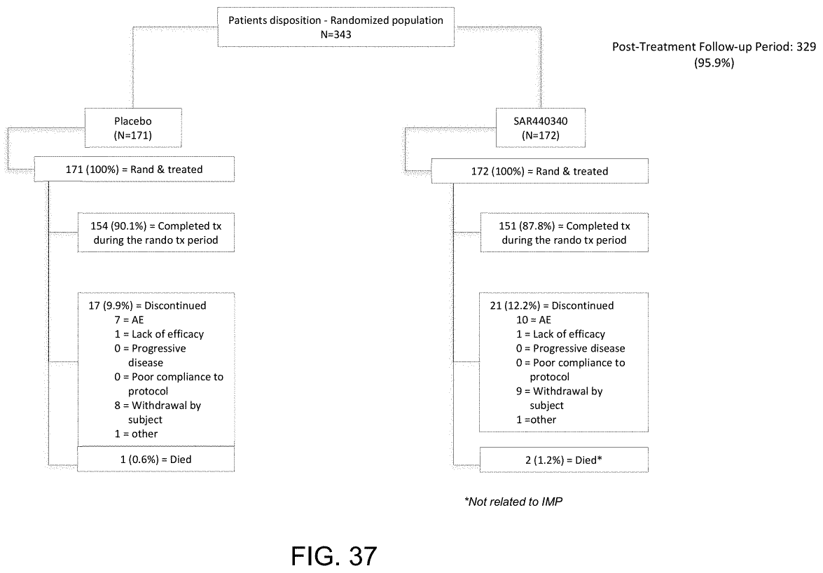

A schematic of the patients selected is shown in FIG. 37. Approximately 340 patients were randomized in this study (170 patients per arm) of which approximately 50% of patients presented with a blood eosinophil count of ≥250/mm³ and about 50% of patients with a blood eosinophil count of <250/mm³.

A summary of key inclusion and exclusion criteria is presented in Table 2 below.

TABLE 2
Key Inclusion and Exclusion Criteria.
Key Inclusion Criteria

| | |
|---|---|
| Age/Sex | Adults, 40-75 years, females and males |
| COPD history | COPD for at least 12 months based on Global |
| | Initiative for Chronic Obstructive Lung |
| | Disease [GOLD] definition |
| Background COPD | Double therapy: LABA + LAMA or ICS + LABA |
| | or ICS + LAMA (OR) Triple therapy: ICS + |
| tx | LABA + LAMA |
| Smoking | Current or former smokers with a smoking |
| | history of ≥10 pack-years |
| Post-BD FEV1 | FEV1 ≥30% but <80% of predicted normal |
| | at Visit 1 and 2 |
| CAT | ≥10 at Visit 1 and 2 |
| # of prior severe | ≥2 moderate (req OCS +/− Abx) or ≥1 severe |
| exacerbations | AECOPD (req hospitalization) in past 1 year |
| | prior to screening |
| Screening | All eosinophil levels, stratification by <250 |
| Eosinophils | and ≥250 (Visit #1) |

Key Exclusion Criteria

Current diagnosis of asthma (past/history of
asthma OK)
Diagnosis of alpha-1 anti-trypsin deficiency
Advanced COPD with need for chronic
(>15 hrs/day) oxygen support
Concomitant severe disease which use of ICS
or LABA is contraindicated
Use of systemic corticosteroids within 1 month
of Visit 1, or 4+ sources of IV glucocorticoids
within 6 months
Moderate or severe AECOPD event within
4 weeks prior to screening; LRTI within 4
weeks of screening or during screening
Prior history or planned pneumonectomy or
LVR surgery
Patients participating in the acute phase of
pulmonary rehabilitation program (<4 weeks)
prior to screening

Participants that were 40 to 75 years of age inclusive, were eligible to participate in the study. Participants were eligible to be included in the study only if all of the following criteria applied: (1) Participants with a diagnosis of COPD for at least 1 year (based on the Global Initiative for Chronic Obstructive Lung Disease (GOLD) definition, found in the Global Initiative for Chronic Obstructive Lung Disease. Global strategy for the diagnosis, management, and prevention of chronic obstructive pulmonary disease (2017 report). (Cited 8 Mar. 2018). Available from the website goldcopd.org/wp-content/uploads/2016/12/wms-GOLD-2017-Pocket-Guide.pdf); (2) Participants with moderate-to-severe COPD (post-bronchodilator FEV1/forced vital capacity [FVC]<70% and post-bronchodilator FEV1% predicted <80%, but ≥30%) at visits 1 and 2; (3) Participants with COPD Assessment Test (CAT) score ≥10 at screening visit 1 and visit 2/randomization; (4) Participants with reported history of signs and symptoms of chronic bronchitis (chronic productive cough for 3 months in the year up to screening in a patient in whom other causes of chronic cough (e.g., gastroesophageal reflux, chronic rhinosinusitis, bronchiectasis) have been excluded); (5) Participants with a documented history (e.g., medical record verification) of ≥2 moderate exacerbations or ≥1 severe exacerbation within the year prior to screening, wherein a moderate exacerbation is defined as an AECOPD requiring systemic corticosteroids (oral, intravenous, or intramuscular) and/or treatment with antibiotics (however, use of antibiotics alone does not qualify as a “moderate exacerbation” unless documentation is available that use of antibiotics was necessary for treatment of worsening symptoms of COPD), and a severe exacerbation is defined as an AECOPD that required a hospitalization; (6) Participants with Standard of Care background therapy, for 3 months prior to Visit 2/Randomization and at a stable dose for at least 1 month prior to the screening, including either double therapy (LABA+LAMA or ICS+LABA or ICS+LAMA) or triple therapy (ICS+LABA+LAMA); (7) Current or former smokers with a smoking history of >10 packs-year; (8) Body mass index (BMI) ≥18.0 kg/m² (inclusive); (9) Male or Female; and (10) Capable of giving signed informed consent.

Patients who met all the above inclusion criteria were screened for the following exclusion criteria: (1) Clinically significant abnormal electrocardiogram (ECG) at visit 1 that may affect the conduct of the study in the judgment of the investigator; (2) Concomitant severe diseases or diseases for which the use of ICS (e.g., active pulmonary tuberculosis) or LABA are contraindicated (e.g., diagnosis of a history of significant cardiovascular diseases, insulin-dependent diabetes mellitus, hyperthyroidism, thyrotoxicosis, pheochromocytoma, hypokalemia); (3) Use of injectable glucocorticosteroids or oral systemic glucocorticosteroids within 1 month prior to visit 1/Screening or more than 4 courses of IV glucocorticosteroids within the 6 months prior to visit 1; (4) Participants receiving medications or therapy that are prohibited as concomitant medications including: Systemic steroids (except when used to treat exacerbations, Note: one short-term course of systemic corticosteroids (up to 6 days) is permitted in 24 weeks, when medically necessary for reasons not related to AECOPD, e.g., in the case of severe poison ivy exposure; PDE-4 inhibitor such as roflumilast; methylxanthines (theophylline, aminophyllines); leukotriene receptor antagonists or leukotriene synthesis inhibitors; lipoxygenase inhibitors; anti-IL5 mAb (e.g., benralizumab; mepolizumab); anti-IgE therapy (e.g., omalizumab); anti-IL4R mAb (e.g., dupilumab); systemic immunosuppressants (e.g., methotrexate, any anti-TNF mAbs, B and/or T cell targeted immunosuppressive therapies); bronchial thermoplasty; intravenous immunoglobulin (IVIG) therapy; live attenuated vaccines; β-adrenergic receptor blockers (except for a selective β-1 adrenergic receptor blocker used with dose stable 1 month prior to visit 1); COPD relievers other than salbutamol/albuterol, levosalbutamol/levalbuterol or ipratropium (their use is not recommended during the study period, in case of use in exceptional circumstances (e.g., prescribed by a physician not participating in the study), their use is documented in the patient's file and reported in the eCRF); other investigational drugs. The following concomitant medications were permitted during the study: antihistamines, ocular, intranasal, and topical corticosteroids; (5) A participant with a history of clinically significant renal, hepatic, cardiovascular, metabolic, neurologic, hematologic, ophthalmologic, respiratory, gastrointestinal, cerebrovascular or other significant medical illness or disorder which, in the judgment of the Investigator, could interfere with the study or require treatment that might interfere with the study.

A specific example includes but is not limited to poorly controlled insulin-dependent diabetes; (6) Participants with bronchial thermoplasty procedure (up to 3 years prior to Visit 1); (7) Exclusion related to tuberculosis (TB): Active TB or a history of incompletely treated TB, confirmed quantiferon-positive patients (no active disease) are excluded from the study unless the following conditions are met: Patients with a history of prior documented completed chemoprophylaxis for latent tuberculosis infection (with a treatment regimen as per local guidelines) or treatment of active TB infection, and as obtained consultation with a specialist to rule out or treat active TB infection, suspected extrapulmonary TB infection, or patients at high risk of contracting TB, such as close contact with individuals with active or latent TB; (8) A current diagnosis of asthma according to the Global Initiative for Asthma (GINA) guidelines (Global Initiative for Asthma. Global Strategy for Asthma Management and Prevention (GINA 2018). 2018. [Cited 8 Mar. 2018]. Available from the website: ginasthma.org/2018-gina-report-global-strategy-for-asthma-management-and-prevention); (9) Significant pulmonary disease other than COPD (e.g., lung fibrosis, sarcoidosis, interstitial lung disease, pulmonary hypertension, bronchiectasis, eosinophilic granulomatosis with polyangiitis, significant sleep apnea on Bilevel Positive Airway Pressure, etc.) or another diagnosed pulmonary or systemic disease associated with elevated peripheral eosinophil counts; (10) Diagnosis of α-1 anti-trypsin deficiency; (11) Advanced COPD with need for chronic (>15 hours/day) oxygen support; (12) Participant with a moderate or severe AECOPD event within 4 weeks prior to screening; (13) A participant who has experienced an upper or lower respiratory tract infection within 4 weeks prior to screening/visit 1 or during the screening period; (14) Prior history of or planned pneumonectomy or lung volume reduction surgery; (15) Participants with a history of a systemic hypersensitivity reaction to a mAb drug; (16) Anti-IgE therapy (e.g., omalizumab (XOLAIR®)) within 130 days prior to visit 1 or any other biologic therapy (including anti-IL5 mAb, e.g. benralizumab (FASENRA®) or mepolizumab (NUCALA)) for asthma or systemic immunosuppressant (e.g., methotrexate) to treat other inflammatory disease or autoimmune disease (e.g., rheumatoid arthritis, inflammatory bowel disease, primary biliary cirrhosis, systemic lupus erythematosus, multiple sclerosis, etc.) and other diseases, within 2 months or 5 half-lives prior to Visit 1, whichever is longer; (17) Current history of substance and/or alcohol abuse; (18) Inability to follow the procedures of the study (e.g., due to language problems, psychological disorders) or unable to read, understand and fill a questionnaire or use an electronic diary without any help; (19) Exposure to another investigative drug (small molecules as well as mAbs, including dupilumab) within a time period prior to visit 1 that is <5 PK half-lives of the antibody. In case the half-life is not known, then the minimum interval since the exposure to the prior investigative antibody is 6 months. The minimum interval since exposure to any other (non-antibody) investigative study medication is 30 days prior to visit 1; (20) Patients who are participating in the acute phase of a pulmonary rehabilitation program, i.e., who started rehabilitation <4 weeks prior to screening (Note: patients in the maintenance phase of a rehabilitation program can be included); (21) Clinically relevant (based on study investigator's judgment) abnormal laboratory values suggesting an unknown disease and requiring further evaluation; (22) Participants previously treated in any clinical trial of SAR440340; (23) Participant is the investigator, or any sub-investigator, research assistant, pharmacist, study coordinator, other staff or relative thereof directly involved in the conduct of the study; (24) Prisoners and participants who are legally institutionalized; (25) Known allergy to doxycycline or related compounds, or known allergy to SAR440340 excipients; (26) Females who are lactating, breastfeeding or who are pregnant; (27) Women of childbearing potential (premenopausal female biologically capable of becoming pregnant) who are not protected by one of the acceptable forms of effective contraception or who do not have a confirmed negative serum β-human chorionic gonadotropin (β-hCG) test at visit 1 and a negative urine pregnancy test prior to visit 2/randomization (Post-menopausal women (defined as at least 12 consecutive months without menses) are not required to use additional contraception); Male participants with female partners of childbearing potential are not eligible to participate unless they agree to ONE of the following: are abstinent from penile-vaginal intercourse as their usual and preferred lifestyle (abstinent on a long term and persistent basis) and agree to remain abstinent or agree to use a male condom plus partner use of a contraceptive method with a failure rate of <1% per year when having penile-vaginal intercourse with a woman of childbearing potential who is not currently pregnant; men with a pregnant or breastfeeding partner must agree to remain abstinent from penile-vaginal intercourse or use a male condom during each episode of penile penetration; (28) Diagnosed active parasitic infection (helminths), suspected or high risk of parasitic infection, unless clinical and (if necessary) laboratory assessments have ruled out active infection before randomization; (29) History of human immunodeficiency virus (HIV) infection or positive HIV 1/2 serology; (30) Known or suspected history of immunosuppression, including history of invasive opportunistic infections (e.g., TB, histoplasmosis, listeriosis, coccidioidomycosis, penumocystosis, aspergillosis), despite infection resolution; or unusually frequent, recurrent or prolonged infections, per Investigator's judgment; (31) Live, attenuated vaccinations within 12 weeks prior to visit 1 or planned live, attenuated vaccinations during the study; (32) Patients with autoimmune disease or patients using systemic immunosuppressive therapy for autoimmune disease (e.g., rheumatoid arthritis, inflammatory bowel disease, primary biliary cirrhosis, systemic lupus erythematosus, multiple sclerosis, etc.) or patients with high titer autoantibodies at screening who are suspected of having high risk for developing autoimmune disease at the discretion of the Investigator or the Sponsor; (33) Patients with cardiovascular diseases/conditions including unstable ischemic heart disease, including acute myocardial infarction within past 1 year or unstable angina in the last 6 months, cardiac arrhythmias including paroxysmal (e.g., intermittent) atrial fibrillation are excluded. Patients with persistent atrial fibrillation as defined by continuous atrial fibrillation for at least 6 months and controlled with a rate control strategy (i.e., selective R blocker, calcium channel blocker, pacemaker placement, digoxin or ablation therapy) and stable appropriate level of anticoagulation for at least 6 months may be considered for inclusion, cardiomyopathy, as defined by stage III-IV (New York Heart Association) cardiac failure, or other relevant cardiovascular disorder that in the investigator's judgment may put the patient at risk or negatively affect the study outcome, and uncontrolled hypertension (i.e., systolic blood pressure [BP]>180 mm Hg or diastolic BP >110 mm Hg despite use of anti-hypertensive therapy); (34) Hepatitis B and/or C serologies indicative of active or chronic infection; (35) Any prior history of malignancy or active malignancy, including lymphoproliferative diseases (except successfully-treated carcinoma in-situ of the cervix, non-metastatic squamous cell or basal cell carcinoma of the skin) within 5 years prior to visit 2; (36) Clinically significant laboratory tests at screening/visit 1 including alanine transaminase (ALT) or aspartate transaminase (AST) >3 times upper limit of normal range (ULN), hemoglobin <10 g/dL for male and <9 g/dL for female, neutrophils <1.5 K/mm³ (<1 K/mm³ for those of African descent), platelets <100 K/mm³, or creatinine ≥150 mol/L; (37) Patients on macrolide (e.g., azithromycin) therapy, unless on stable therapy for ≥1 year; (38) Patients on PDE-4 inhibitors (roflumilast) or leukotriene blockers (montelukast, Singulair, etc.); and (40) Despite screening of the patient, enrollment/randomization is stopped at the study level.

Only patients who met all of the inclusion criteria, and none of the exclusion criteria, were included in the study.

Baseline demographics of the study participants are presented in Table 3. The demographics were balanced between the treatment and placebo groups, and females represented greater than 40% of the population.

TABLE 3
Baseline Demographics.

| | | N = 171 | N = 172 | N = 343 |
|---|---|---|---|---|
| Category | Data | Placebo | SAR440340 | All |
| Age | Mean (SD) | 64.0 (6.5) | 63.7 (6.8) | 63.9 (6.7) |
| (years) | | | | |
| Sex | Female | 76 (44.4) | 73 (42.4) | 149 (43.4) |
| [n (%)] | | | | |
| Race | White | 169 (98.8) | 170 (98.8) | 339 (98.8) |
| [n (%)] | Black or | 1 (0.8) | 1 (0.6) | 2 (0.6) |
| | African | | | |
| | American | | | |
| | Asian | 0 | 1 (0.6) | 1 (0.3) |
| Ethnicity | Hispanic | 33 (19.3) | 37 (21.5) | 70 (20.4) |
| | or Latino | | | |
| [n (%)] | Not Hispanic | 137 (80.1) | 135 (78.5) | 272 (79.3) |
| | or Latino | | | |
| Weight | Mean (SD) | 79.49 | 78.69 | 79.09 |
| (kg) | | (18.52) | (19.34) | (18.91) |
| BMI group | <25 | 53 (31.0) | 53 (30.8) | 106 (30.9) |
| (kg/m2) | ≥25 - <30 | 63 (36.8) | 68 (39,5) | 131 (38.2) |
| [n (%)] | ≥30 - <35 | 31 (18.1) | 31 (18.0) | 62 (18.1) |
| | ≥35 | 24 (14.0) | 20 (11.8) | 44 (12.8) |
| Region | East Europe | 73 (42.7) | 75 (43.6) | 148 (43.1) |
| [n (%)] | Latin | 37 (21.6) | 42 (24.4) | 79 (23.0) |
| | American | | | |
| | Western | 61 (35.7) | 55 (32.0) | 116 (33.8) |
| | countries | | | |

### D. Study Treatments

### Investigational Medicinal Product

As shown in Table 4 below, the Investigational Medicinal Product (IMP) includes SAR440340 and placebo for subcutaneous injection during the course of the study.

In the group that received the IMP, sterile SAR440340 was provided in one 20 mL vial containing 287 mg of lyophilisate drug product. One vial of lyophilisate drug product (287 mg) or placebo was reconstituted with 2.5 mL of sterile water for injection resulting in 2.9 mL of 100 mg/mL SAR440340 or placebo. A volume of 1.5 mL per injection was withdrawn from the vial. Patients received 2 subcutaneous injection injections per dose. Subcutaneous injection sites alternated between the upper thighs, 4 quadrants of the abdomen or the upper arms, so that the same site is not injected twice during consecutive visits The investigational medicinal product (IMP) or placebo was administered every 14±3 days (q2w) for 24-52 weeks.

TABLE 4
Overview of Investigational Medicinal Products Administered.

| Investigational Medicinal | | |
|---|---|---|
| Product name | SAR440340 | Placebo |
| Dosage formulation | Sterile 5AR440340 | Sterile placebo |
| | will be provided | will be provided |
| | in one 20 mL vial | in one 20 mL |
| | containing 287 mg | vial containing |
| | of lyophilisate drug | lyophilisate |
| | product. | placebo. |
| | One vial of | One vial of |
| | lyophilisate drug | lyophilisate is |
| | product (287 mg) | reconstituted by |
| | is reconstituted by | an unmasked site |
| | an unmasked site | pharmacist or |
| | pharmacist or | designee (not |
| | designee (not | involved in any |
| | involved in any | study related |
| | study related | assessments/ |
| | assessments/activities | activities except |
| | except preparation | preparation of IMP) |
| | of IMP) with 2.5 mL | with 2.5 mL of |
| | of sterile water for | sterile water for |
| | injection resulting in | injection resulting |
| | 2.9 mL of 100 mg/mL | in 2.9 mL IMP. A |
| | SAR440340. | volume of 1.5 mL |
| | A volume of 1.5 mL | per injection will |
| | per injection will | be withdrawn from |
| | be withdrawn from | the vial. Patients |
| | the vial. Patients will | will receive 2 |
| | receive 2 injections | injections per dose. |
| | per dose. | |
| Unit dose strength(s)/ | 300 mg | Not applicable |
| Dosage level(s) | | |
| Route of administration | Subcutaneous | Subcutaneous |
| | injection sites | injection sites |
| | should | should alternate |
| | alternate between | between the upper |
| | the upper thighs, | thighs, 4 quadrants |
| | 4 quadrants of the | of the abdomen or |
| | abdomen or the | the upper arms, so |
| | upper arms, so that | that the same site is |
| | the same site is not | not injected twice |
| | injected twice during | during consecutive |
| | consecutive visits | visits |
| Dosing instructions | 14 ± 3 days (q2w) | 14 ± 3 days (q2w) |
| Packaging and labeling | SAR440340 will be | Matched placebo |
| | supplied in a glass | will be supplied |
| | vial packed in a kit | in a glass vial |
| | box. Each kit box | packed in a kit |
| | will be labeled | box. Each kit |
| | as required | box will be |
| | per country | labeled as required |
| | requirement. | per country |
| | | requirement. |

### Non-Investigational Medicinal Products

At screening visit 1, all patients were on standard of care background therapy for 3 months prior to visit 2/randomization, and at a stable dose for at least 1 month prior to the screening/visit 1, including either double therapy (LABA+LAMA or ICS+LABA or ICS+LAMA) or triple therapy (ICS+LABA+LAMA).

The formulation of the background therapy was dry powder inhaler (DPI), metered dose inhaler (MDI) or pocket nebulizer. The route of administration of the background therapy was oral inhalation. The dose regimen of the background therapy was as prescribed.

Throughout the study, patients were to continue their established background therapy for COPD. Patients are willing to stay on their established background medication for COPD throughout the duration of the study. After successful management of an acute exacerbation of COPD (e.g., with oral corticosteroids and/or antibiotics), all efforts should have been made to resume the initial background COPD treatment regimen if in the investigator's opinion this is medically acceptable. After 1 severe or 2 moderate exacerbations of COPD, dose adjustments in background therapy were to be permitted for symptom control and as needed for the remainder of the trial period.

Patients could use albuterol/salbutamol or levalbuterol/levosalbutamol (including ipratropium or ipratropium/short-acting Ragonists [SABA] combinations) as reliever medication as needed during the study. Nebulizer solutions could be used as an alternative delivery method.

The formulation of the reliever medication included dry powder inhaler (DPI), metered dose inhaler (MDI) or pocket nebulizer. The route of administration of the reliever medication was oral inhalation. The dose regimen of the background therapy was as prescribed.

### Efficacy Assessments

The Severity of COPD exacerbations were defined by the protocol. “Moderate exacerbations” were recorded by the Investigator and defined as AECOPD that required either systemic corticosteroids (such as intramuscular, intravenous or oral) and/or antibiotics. “Severe exacerbations” were recorded by the Investigator and defined as AECOPD requiring hospitalization, emergency medical care visit or resulting in death. In addition to these protocol-defined exacerbations of COPD, clinical signs and symptoms of exacerbations of COPD were captured in the eCRF (including, but not limited to increase in dyspnea, increase in wheezing, increase in cough, increase in sputum volume and/or increase in sputum purulence).

Exacerbations of COPD were treated as deemed necessary by the investigator. After successful management of an acute exacerbation of COPD (e.g., with oral corticosteroids and/or antibiotics), all efforts were made to resume the initial background COPD treatment regimen if in the investigator's opinion this is medically acceptable. After 1 severe or 2 moderate exacerbations of COPD, dose adjustments in background therapy were permitted for symptom control and as needed for the remainder of the trial period.

Spirometry at clinical site visits should be performed in accordance with the European Respiratory Society (ERS)/American Thoracic Society (ATS) guidelines (Miller M R, Hankinson J, Brusasco V, Burgos F, Casaburi R, Coates A, et al. Standardization of spirometry. Series “ATS/ERS TASK FORCE: Standardization of Lung Function Testing” Edited by Brusasco V, Crapo R, and Viegi G. Eur Respir. J. 2005 August; 26(2):319-38) and prior to administration of investigational product. For pre-bronchodilator measured parameters, including FEV1, peak expiratory flow (PEF), FVC and forced expiratory flow (FEF) 25%-75%, spirometries were performed after a washout period of bronchodilators according to their action duration, for example, withholding the last dose of salbutamol/albuterol or levosalbutamol/levalbuterol for at least 6 hours, withholding the last dose of LABA for at least 12 hours (ultra-long acting LABA like vilanterol should be withheld for at least 24 hours), withholding the last dose of ipratropium for at least 8 hours and withholding the last dose of LAMA for at least 24 hours. This was verified before performing the measurements. When both pre- and post-bronchodilator spirometry was assessed, the post-bronchodilator spirometry was performed consistent with the mechanism of action of reliever (i.e., 30 minutes for albuterol or other SABA). At all visits, spirometry was performed preferably in the morning; afternoon/evening was allowable in the exceptional circumstance when morning spirometry could not be performed; spirometry was done at approximately the same time at each visit throughout the study. Current smokers were reminded not to smoke for at least 1 hour before spirometry. The same spirometer and standard spirometric techniques, including calibration, were used to perform spirometry at all visits and, whenever possible, the same person performed the measurements. Three measurements fulfilling the ATS acceptability and repeatability criteria were obtained at every visit, if possible.

Fractional exhaled nitric oxide (FeNO) was analyzed using a NIOX instrument (Aerocrine AB, Solna, Sweden), or similar analyzer using a flow rate of 50 mL/s, and reported in parts per billion (ppb). This assessment was conducted prior to spirometry and following a fast of at least 1 hour.

Optional assessments of actigraphy (sleep and activity) and home spirometry were also included. Patients were issued an actigraphic wristband and asked to wear it continuously (including at night) throughout three monitoring periods, including at night. The actigraphic data was used to measure sleep parameters and daytime activity. The actigraph was worn during the screening period as well as two monitoring periods during the treatment phase. Data from the device was uploaded to a computer at each clinic visit following a monitoring period. Patients received documented in-clinic training for use of ambulatory at-home spirometry during screening. During the study, patients were required to use at-home spirometry with electronic data storage to measure FEV1. Patients were instructed to perform expiratory flow maneuvers as described in the study manual at least twice daily between 06:00 and 12:00 hour and between 18:00 and 24:00 hour during the screening period and for 2 week intervals during the treatment and follow-up period.

A subset of study sites was selected to perform evaluations of induced sputum, and patients at these selected sites had the option to participate in this assessment. Sputum induction is a relatively noninvasive method to obtain sputum for cell or fluid phase inflammatory indices, culture or cytology. It is performed with an aerosol of normal or hypertonic saline generated by an ultrasonic nebulizer. As this aerosol is a potential bronchoconstrictive stimulus, it is made safe by pretreatment with salbutamol and inhalation in a dose-response manner.

At screening (visit 1), patients were issued an electronic diary. Patients were instructed on the use of the device, and written instructions on the use of the electronic device was provided to the patients. Recorded information was downloaded from this device on the other indicated days. On a daily basis during screening and treatment, the patient used an electronic diary to: respond to the COPD symptom scale questions of the EXACT tool, record the daily use of COPD reliever medication, and record use of systemic corticosteroids and/or antibiotics taken for COPD exacerbation. The electronic diary was used for patient reported outcome questionnaires. These questionnaires are described below.

### COPD Assessment Test (CAT™)

The CAT™ is a new questionnaire that is designed for patients with COPD to measure the effects of the disease on their quality of lives. The CAT™ is an 8-item self-administered questionnaire which has been developed for use in routine clinical practice to measure the health status of patients with COPD.

The CAT™ score ranges from 0 to 40, a higher score indicating a higher impact on health status. The test is about cough, phlegm, chest tightness, dyspnea, activity limitation, confidence, sleep and energy. Patients scored questions from 1-5 according to their own feelings about the disease (1=I am very happy; 5=I am very sad).

St. George's Respiratory Questionnaire (SGRQ)

The St. George's Respiratory Questionnaire (SGRQ) is a 50-item questionnaire designed to measure and quantify health-related health status in adult patients with chronic airflow limitation. A global score ranges from 0 to 100. Scores by dimension are calculated for three domains: symptoms, activity and impacts (psycho-social) as well as a total score. Lower score indicates better quality of life (QoL).

The first part (“symptoms”) evaluates symptomatology, including frequency of cough, sputum production, wheeze, breathlessness and the duration and frequency of attacks of breathlessness or wheeze. The second part has two components: “activity” and “impacts.” The “activity” section addresses activities that cause breathlessness or are limited because of breathlessness. The “impacts” section covers a range of factors including influence on employment, being in control of health, panic, stigmatization, the need for medication, side effects of prescribed therapies, expectations for health and disturbances of daily life. The recall period of the questionnaire is over the past 4 weeks.

Psychometric testing has demonstrated its repeatability, reliability and validity. Sensitivity has been demonstrated in clinical trials. A minimum change in score of 4 units was established as clinically relevant after patient and clinician testing. The SGRQ has been used in a range of disease groups including asthma, COPD and bronchiectasis.

### Exacerbations of Chronic Obstructive Pulmonary Disease Tool (EXACT)

The EXACT total score measures symptoms of acute bacterial exacerbations of chronic bronchitis-COPD (ABECB-COPD), i.e., an acute, sustained, and worsening of signs and symptoms beyond day-to-day variability. The instrument's total score is made up of a total of 14 items representing the following domains:

Breathlessness (5 items),

Cough and sputum (2 items),

Chest symptoms (3 items),

Difficulty bringing up sputum (1 item),

Tired or weak (1 item),

Sleep disturbance (1 item), and

Scared or worried (1 item).

The EXACT is a daily diary, completed each evening before bedtime. The instrument was developed with e-diary administration in mind, with cognitive interviews performed with paper pen booklet and personal digital assistant (PDA) to document respondent understanding in either mode and user acceptance of the PDA.

### Euro Quality of Life-5 Dimension Questionnaire (EQ-5D)

EQ-5D-5 L is a standardized health-related QoL questionnaire developed by the EuroQol Group in order to provide a simple, generic measure of health for clinical and economic appraisal. EQ-5D is designed for self-completion by patients.

### Safety Assessments

The same safety assessments will be applied to treatment and placebo groups. Adverse events, including SAEs and adverse events of special interest (AESI), were collected at every visit.

A complete physical examinations included skin, nasal cavities, eyes, ears, respiratory, cardiovascular, gastrointestinal, neurological, lymphatic, and musculoskeletal systems. All deviations from normal were recorded, including those attributable to the patient's disease.

Vital signs, including systolic and diastolic BP (mm Hg), pulse rate (beats per minute), body temperature (° C.), and respiratory rate were measured at screening, baseline and every subsequent on-site visit. Height (cm) will be measured at Screening (visit 1) only. Body weight (kg) was measured at screening (visit 1) and EOT/EOS visits.

Recording of a standard 12-lead electrocardiogram (ECG) was performed at the site. At randomization visits, ECGs were performed prior to investigational product administration. A minimum of 3 complexes in an appropriate lead (lead II) were averaged to determine the PR-interval, QT/QTc-interval, QRS-complex and heart rate was measured for each ECG.

### Smokers

Smoking status was determined for the subjects. Smoking habits only concerned tobacco (e.g., cigarettes, cigars, pipes). The use of chewing or smoking tobacco was not used for reporting. A score of “never” was given is a subject smoked less than one cigarette per day on average, and the subject was considered a non-smoker. A score of “current” was given if the subject smoked at least one cigarette, as a mean, per day during the past 7 days. A score of “former” was given if the subject previously smoked but stopped at least 8 days prior to the study. Th duration of smoking cessation in the study was as low as approximately 1.2 months and as high as 56.1 years, with a mean of 11.80 years and a median of 9.92 years.

### Baseline Disease Characteristics

COPD-specific baseline disease characteristics are presented in Table 5 below. The SAR440340 treated and placebo groups were balanced in terms of COPD disease-specific characteristics.

TABLE 5
Baseline Disease Characteristics, COPD-Specific.

| | | N = 171 | N = 172 | N = 343 |
|---|---|---|---|---|
| Category [symbol] | Data | Placebo | SAR440340 | All |
| Age at diagnosis of | Mean | 55.5 | 55.4 | 55.4 |
| COPD (years) | (SD) | (8.0) | (78) | (7.9) |
| Age group at | ≥18 - | 3 | 3 | 6 |
| diagnosis of COPD | <40 | (1.8) | (1.7) | (1.71) |
| (years) [n (%)] | ≥40 - | 109 | 1.13 | 222 |
| | <60 | (63.7) | (65.7) | (64.7) |
| | ≥60 - | 59 | 56 | 115 |
| | <75 | (34.5) | (32.6) | (33.5) |
| Time since diagnosis | Mean | 9.31 | 9.13 | 9.22 |
| of COPD at | (SD) | (6.15) | (5.38) | (5.77) |
| randomization (years) | | | | |
| Time since as | Mean | 5.0 | 5.6 | 5.3 |
| COPD exacerbation | (SD) | (2.8) | (2.8) | (2.8) |
| at randomization | | | | |
| (months) | | | | |
| Smoking | Current | 82 | 74 | 156 |
| History | | (48.0) | (43.0) | (45.5) |
| [n (%)] | Former | 89 | 98 | 187 |
| | | (52.0) | (57.0) | (54.5) |
| Baseline post-BD | Mean | 46.29 | 45.40 | 45.84 |
| FEV1/FVC ratio (%) | (SD) | (10.66) | (9.64) | (10.15) |
| Baseline pre- | Mean | 1.28 | 1.30 | 1.29 |
| BD FEV1 (L) | (SD) | (0.44) | (0.4) | (0.45) |
| Baseline pre-BD FEV1 | Mean | 45.61 | 45.68 | 45.65 |
| percent predicted (%) | (SD) | (12.12) | (12.13) | (12.11) |
| Baseline post- | Mean | 1.38 | 1.41 | 1.39 |
| BD FEV1 (L) | (SD) | (0.46) | (0.47) | (0.46) |
| Baseline post-BD FEV1 | Mean | 49.02 | 49.62 | 49.32 |
| percent predicted (%) | (SD) | (12.18) | (12.34) | (12.25) |
| Baseline CAT score | Mean | 21.66 | 21.89 | 21.78 |
| | (SD) | (5.23) | (5.84) | (5.54) |
| Baseline SGRQ | Mean | 54.07 | 54.45 | 54.26 |
| total score | (SD) | (16.29) | (16.60) | (16.43) |

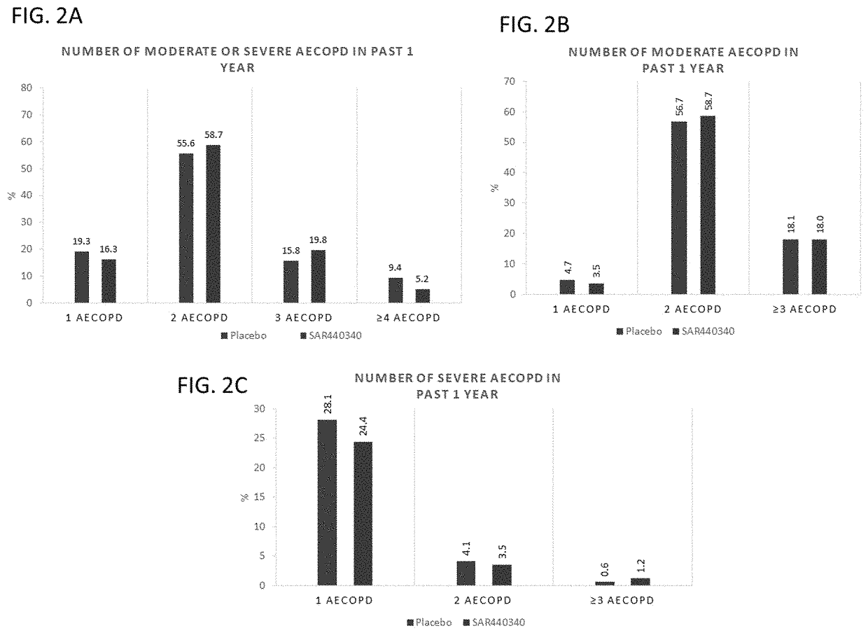

FIG. 2 presents data related to baseline exacerbation history for SAR440340 treated and placebo groups. In FIG. 2A, data is presented for number of moderate or severe AECOPD in the past year with respect to both groups. Data is also presented for number of moderate (FIG. 2B) and severe (FIG. 2C) AECOPD separately. This data shows that both SAR440340 treated and placebo groups were balanced in terms of exacerbation history.

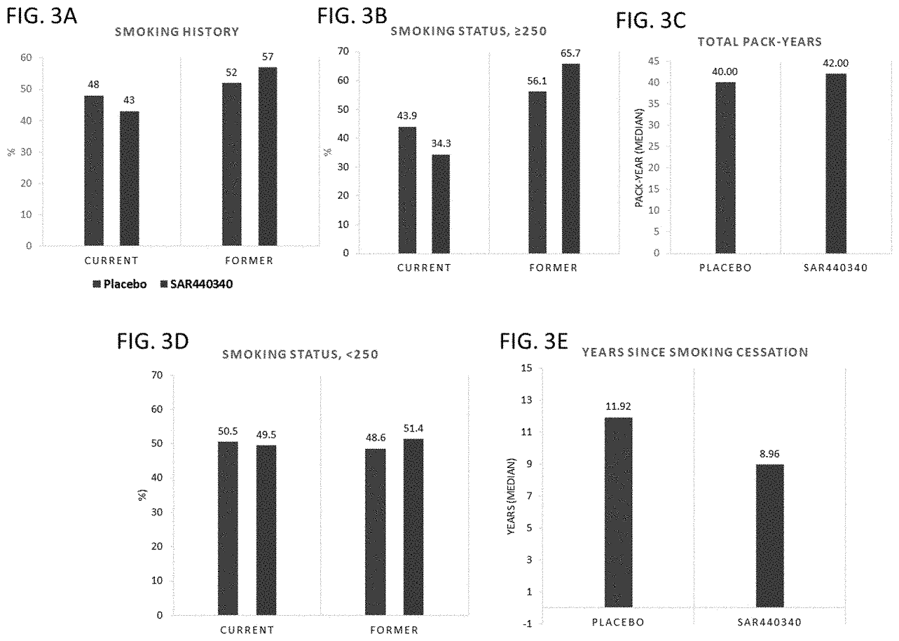

FIG. 3 presents data related to baseline smoking history for SAR440340 treated and placebo groups. In FIG. 3A, data is presented that shows the number and percentage of participants that are current and former smokers in both groups. Data is also presented for subgroups based on eosinophil levels (≥250/μl for high (FIG. 3B) versus <250/μl for low (FIG. 3D)). Additional data is presented that shows the total packs of cigarettes smoked per year (FIG. 3C) and the years since smoking cessation (FIG. 3E) for SAR440340 treated and placebo groups. This data shows that both SAR440340 treated and placebo groups were balanced in terms of smoking history.

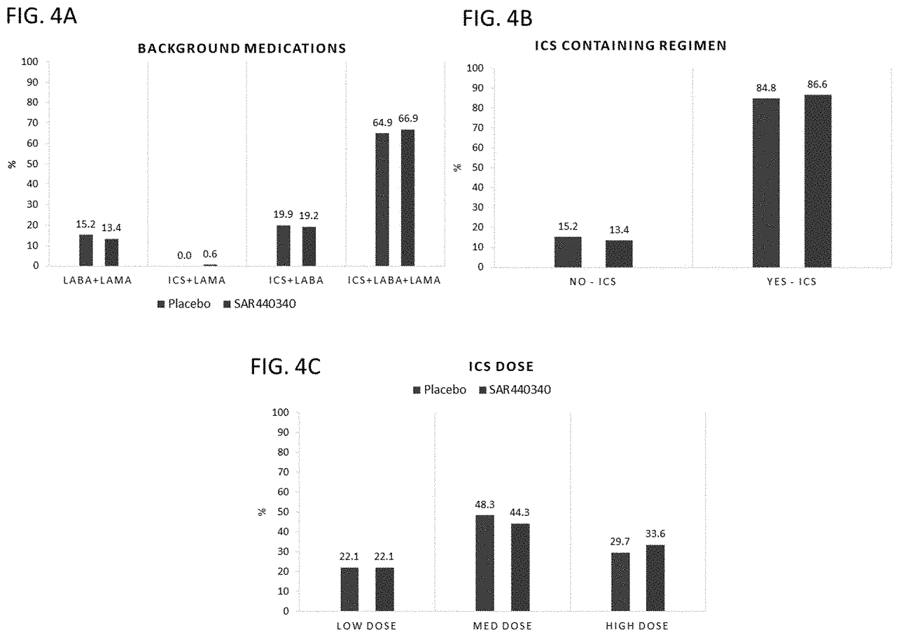

FIG. 4 presents data related to baseline background medications for SAR440340 treated and placebo groups. Data is presented in FIG. 4A related to the number of participants in each of the respective groups taking the following combinations of background medications: LABA+LAMA, ICS+LAMA, ICS+LABA, and ICS+LABA+LAMA. Additionally, data is presented in FIG. 4B showing the number of participants in each of the respective groups with a ICS containing background regimen. Data is also presented in FIG. 4C showing the ICS dose (low, medium, or high) of those participants taking an ICS containing background regimen in the SAR440340 treated and placebo groups. These data show that most patients were on ICS containing regimen.

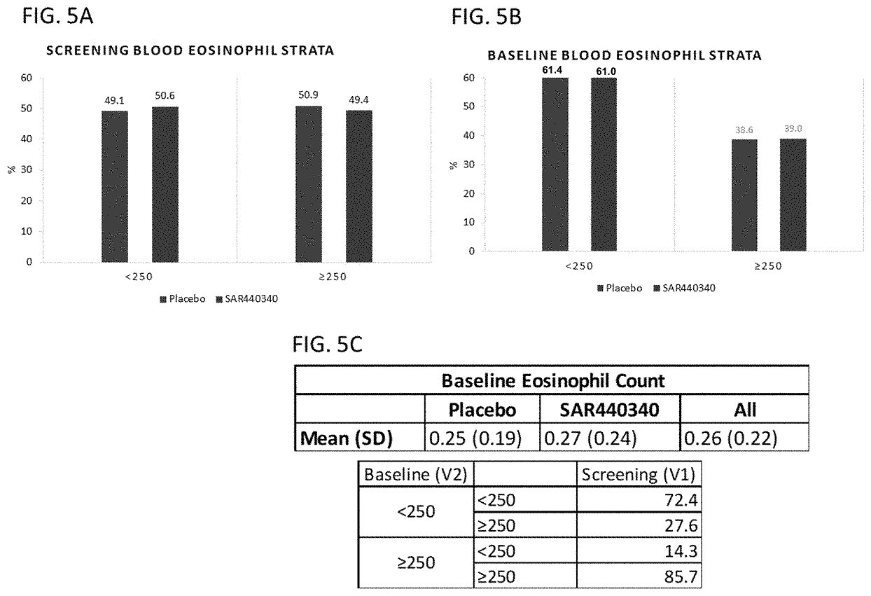

FIG. 5 presents data showing baseline blood eosinophils for SAR440340 treated and placebo groups. Data is presented for subgroups based on eosinophil levels (≥250/μl for high versus <250/μl for low). Data is presented at screening (FIG. 5A) and at baseline (FIG. 5B). This data shows that at screening both eosinophil groups were close to evenly represented for the SAR440340 treated and placebo groups. At baseline, however, both the SAR440340 treated and placebo groups had a higher number of participants with a low eosinophil level.

Study participant baseline biomarker values for participants administered SAR440340 or placebo are shown in Table 6 below. This data shows that the SAR440340- and placebo-administered groups were balanced in terms of blood biomarkers at baseline. Study participant baseline biomarker values for former and current smokers are shown in Table 7 below. These results show that former smokers had higher mean EOS including more patients with EOS greater than 250 and fewer patients with EOS less than 150, higher mean FeNO (note only n=33 had FeNO measured), and lower mean serum IgE levels as compared to the current smoker group.

TABLE 6
Summary of Baseline Disease Characteristics.

| | | N = 171 | N = 173 | N = 343 |
|---|---|---|---|---|
| Category | Data | Placebo | SAR440340 | All |
| Baseline | Mean | 4.67 | 4.80 | 4.74 |
| blood | (SD) | (1.67) | (2.08) | (1.3) |
| neutrophil | | | | |
| (109/L) | | | | |
| | Median | 4.46 | 4.52 | 4.49 |
| Baseline | Mean | 16.1 | 16.4 | 16.3 |
| Total | (SD) | (3.1) | (4.1) | (3.7) |
| IL33 | | | | |
| (pg/mL) | | | | |
| | Median | 15.7 | 15.7 | 15.7 |
| Baseline | Mean | 6805.6 | 7789.3 | 7299.4 |
| sST2 | (SD) | (3147.9) | (6120.8) | (4888.8) |
| (pg/mL) | | | | |
| | Median | 6260 | 6625 | 6390 |
| Baseline | Mean | 3.81 | 3.20 | 3.51 |
| calcitonin | (SD) | (5.06) | (3.72) | (4.45) |
| (pg/mL) | | | | |
| | Median | 1 | 1 | 1 |
| Baseline | Mean | 77219.3 | 79763.4 | 73495.0 |
| PARC | (SD) | (48554.5) | (41815.1) | (45252.0) |
| (pg/mL) | | | | |
| | Median | 66800 | 72100 | 69200 |
| Baseline | Mean | 40.76 | 44.98 | 42.87 |
| eotaxin-3 | (SD) | | | |
| (pg/mL) | | (24.54) | (31.80) | (28.45) |
| | Median | 35.9 | 34.25 | 35.6 |
| Baseline | Mean | 320.43 | 338.98 | 329.73 |
| Total igE | (SD) | | | |
| (kU/L) | | (752.01) | (761.54) | (755 76) |
| | Median | 79.7 | 96.05 | 92.1 |
| Baseline | Mean | 488.8 | 500.6 | 494.7 |
| Fibrinogen | (SD) | (130.2) | (122.6) | (126.4) |
| (mg/dL) | | | | |
| | Median | 481 | 494.5 | 486 |
| Baseline | Mean | 15.0 | 18.8 | 16.5 |
| pre-BD | (SD) | (10.3) | (14.5) | (12.1) |
| FeNO | | | | |
| (ppb) | | | | |
| | Median | 11 | 14 | 14 |
| Baseline | Mean | 16.7 | 16.4 | 16.6 |
| post-BD | (SD) | (11.1) | (11.9) | (11.3) |
| FeNO | | | | |
| (ppb) | | | | |
| | Median | 12.3 | 13.5 | 13.5 |

*Baseline pre-BD FeNO- N = 20, 12.33 for Placebo, SAR440340, and All, respectively ** Baseline post-BD FeNO- N = 20, 12.33 for Placebo, SAR440340, and All, respectively

Baseline pre-BD FeNO, N=20, 12, 33, for placebo, SAR440340 and all, respectively. **Baseline post-BD FeNO, N=20, 12, 33, for placebo, SAR440340 and all, respectively. Blood biomarkers abbreviations: sST2, soluble IL-33 receptor; PARC, pulmonary and activation-regulated chemokine; FeNO, fractional exhaled nitric oxide; pre-BD, pre-bronchodilator; and post-BD, post-bronchodilator.

TABLE 7
Summary of Baseline Biomarkers Among Former and Current Smokers.

| | Former smokers | | | | | |
|---|---|---|---|---|---|---|
| | Placebo | | SAR440340 | | Total | |
| Category | (N = 89) | | 300 mg q2w(N = 98) | | (N = 187) | |
| Mean baseline blood eosiophil count, mean(SD) | 0.27 | (0.21) | 0.28 | (0.23) | 0.28 | (0.22) |
| Mean baseline blood eosinophil count, mean(SD) | 0.43 | (0.24) | 0.45 | (0.25) | 0.44 | (0.24) |
| in ≥250 EOS stratum | | | | | | |
| Mean baseline blood eosinophil count, mean(SD) | 0.16 | (0.06) | 0.15 | (0.05) | 0.15 | (0.05) |
| in <250 EOS stratum | | | | | | |
| Baseline EOS (≥250), n(%) | 37 | (41.6) | 44 | (44.9) | 81 | (43.3%) |
| Baseline EOS (<150), n(%) | 21 | (23.6) | 25 | (25.5) | 46 | (24.6%) |
| Baseline EOS (≥150-<300), n(%) | 41 | (46.1) | 42 | (42.9)) | 83 | (44.4%) |
| Baseline EOS (≥300), n(%) | 27 | (30.3) | 31 | (31.6)) | 58 | (31.0) |
| Baseline pre-BD FeNO (ppb), mean(SD) | 20.9 | (12.1) | 22.8 | (17.7) | 21.7 | (14.2) |
| Baseline post-BD FeNO (ppb), mean(SD) | 23.4 | (12.2) | 17.6 | (10.0) | 20.9 | (11.3) |
| Baseline Total IgE (kU/L), mean (SD) | 303.12 | (824.11) | 252.61 | (441.20) | 276.65 | (650.75) |
| Baseline eotaxin-3 (pg/mL),ean (SD) | 38.24 | (20.97) | 46.36 | (34.36) | 42.50 | (28.99) |
| Baseline PARC (pg/mL),Mean (SD) | 77498.9 | (34395.9) | 87295.9 | (46336.8) | 82633.2 | (41273.8) |
| Baseline Fibrinogen (mg/dL), Mean (SD) | 494.6 | (127.4) | 489.6 | (125.0) | 492.0 | (125.8) |
| Baseline sST2 (pg/mL), mean (SD) | 7137.5 | (3364.7) | 7179.8 | (3297.2) | 7159.7 | (3320.6) |
| Baseline calcitonin (pg/mL) | 3.72 | (5.19) | 3.24 | (3.67) | 3.47 | (4.46) |
| | Current smokers | | | | | |
| | Placebo | | SAR440340 | | Total | |
| Category | (N = 82) | | 300 mg q2w(N = 74) | | (N = 156) | |
| Mean baseline blood eosiophil count, mean(SD) | 0.23 | (0.17) | 0.26 | (0.25) | 0.24 | (0.21) |
| Mean baseline blood eosinophil count, mean(SD) | 0.34 | (0.18) | 0.43 | (0.32) | 0.38 | (0.25) |
| in ≥250 EOS stratum | | | | | | |
| Mean baseline blood eosinophil count, mean(SD) | 0.14 | (0.09) | 0.14 | (0.10) | 0.14 | (0.09) |
| in <250 EOS stratum | | | | | | |
| Baseline EOS (≥250), n(%) | 29 | (35.4) | 23 | (31.1) | 53 | (33.3%) |
| Baseline EOS (<150), n(%) | 30 | (36.5%) | 24 | (32.4%) | 54 | (34.6%) |
| Baseline EOS (≥150-<300), n(%) | 27 | (32.9%) | 30 | (40.5%) | 57 | (36.5) |
| Baseline EOS (≥300), n(%) | 25 | (30.5%) | 20 | (27%) | 45 | (23.8%) |
| Baseline pre-BD FeNO (ppb), mean(SD) | 11.0 | (7.0) | 15.5 | (11.3) | 12.7 | (6.8) |
| Baseline post-BD FeNO (ppb), mean(SD) | 12.2 | (7.9) | 15.4 | (14.1) | 13.4 | (10.4) |
| Baseline Total IgE (kU/L), mean (SD) | 339.21 | (669.59) | 453.36 | (1037.59) | 393.36 | (862.90) |
| Baseline eotaxin-3 (pg/mL),ean (SD) | 43.49 | (27.77) | 43.16 | (28.20) | 43.33 | (27.88) |
| Baseline PARC (pg/mL),Mean (SD) | 76915.9 | (60517.9) | 69787.8 | (32646.0) | 73534.6 | (49280.8) |
| Baseline Fibrinogen (mg/dL), Mean (SD) | 482.5 | (133.7) | 515.2 | (118.6) | 498.0 | (127.4) |
| Baseline sST2 (pg/mL), mean (SD) | 6447.5 | (2871.7) | 8596.5 | (8494.4)) | 7466.9 | (6281.0) |
| Baseline calcitonin (pg/mL) | 3.91 | (4.95) | 3.14 | (3.81) | 3.54 | (4.45) |

### Efficacy

### Primary Efficacy Endpoint

The primary analysis compared the SAR440340 treatment groups to the placebo group. The primary efficacy endpoint was the annualized rate of moderate-to-severe AECOPD over the treatment period.

For the primary efficacy endpoint, AECOPD, a negative binomial regression model was used to assess treatment differences. The model included the total number of events occurring during the treatment period (up to week 52) as the response variable, and the treatment group, the baseline eosinophil strata and region (pooled countries) as covariates. Log-transformed observation duration was the offset variable. Parameters were estimated using the maximum likelihood method with the Newton-Raphson algorithm. Comparison of the annualized event rates between the treatment group and the placebo group were made within this model and the rate ratios and their 95% confidence intervals were estimated. In the case of premature discontinuation of study drug, a secondary analysis included events up to 14 days after the last dose.

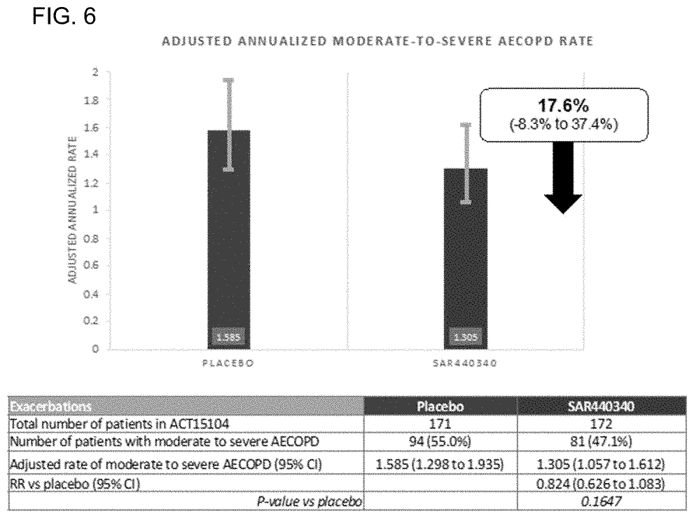

FIG. 6 shows the AECOPD for a combined group of high and low eosinophil subjects either treated with SAR440340 or treated with placebo. These results show that SAR440340 treatment resulted in an about 18% reduction of AECOPD for the combined high and low eosinophil group.

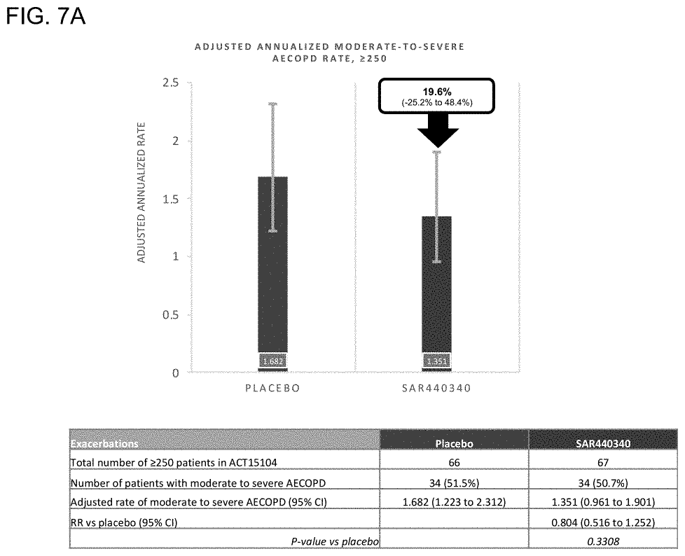

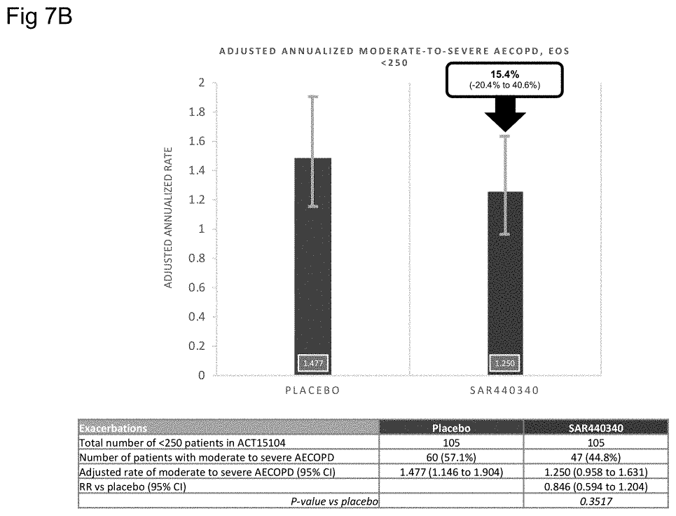

As shown in FIG. 7, subgroup analysis was performed separately by the baseline eosinophil levels (≥250/μl for high (FIG. 7B) versus <250/μl for low (FIG. 7A)). These results show that SAR440340 treatment resulted in similar reduction in AECOPD regardless of baseline eosinophil count. An about 15% reduction in the low eosinophil group versus an about 20% reduction in the high eosinophil group.

### Secondary Efficacy Endpoints

### Time to First Moderate-to-Severe AECOPD

One secondary efficacy endpoint used in the study was time to first moderate-to-severe AECOPD. Time to first moderate or severe AECOPD was analyzed using a Cox regression model with treatment, baseline eosinophil strata, and region (pooled country) as covariates. The Kaplan-Meier (K-M) method was used to estimate the probabilities of first AECOPD at specific time points for each group.

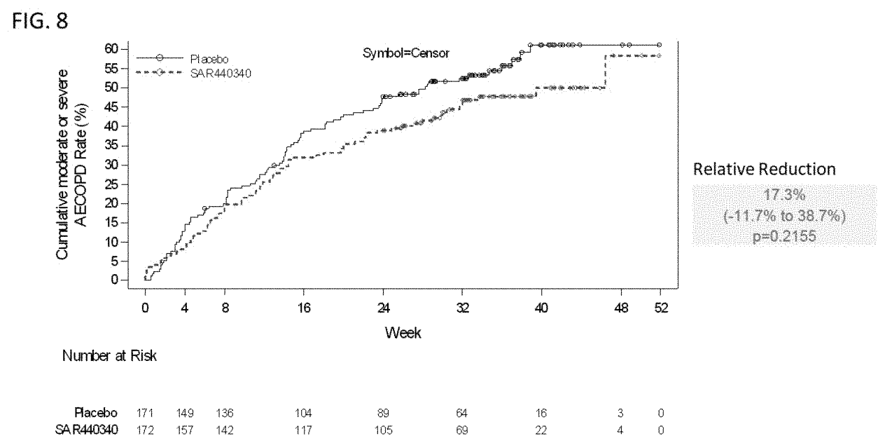

FIG. 8 shows statistical analysis of time to first moderate-to-severe AECOPD in a combined group of high and low eosinophil subjects either treated with SAR440340 or treated with placebo. These results show that SAR440340 treatment resulted in an about 17% reduction in the probability of first AECOPD at a specific time point.

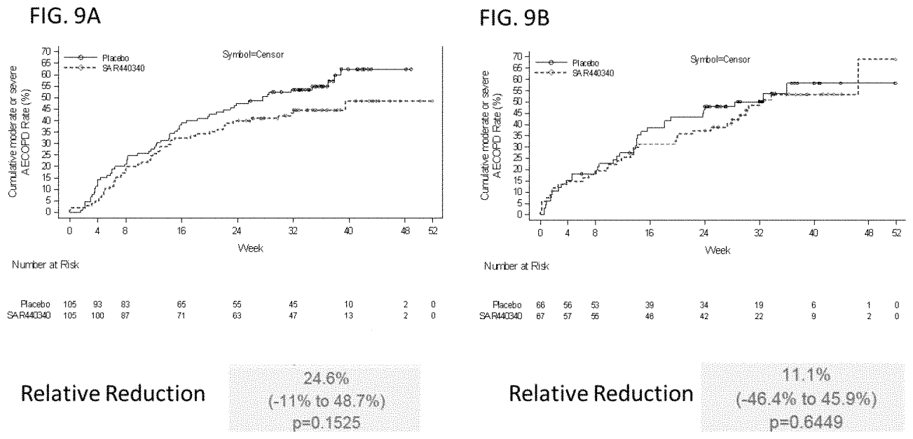

As shown in FIG. 9, subgroup analysis was performed separately by the baseline eosinophil levels (≥250/μl for high EOS (FIG. 9B) versus <250/μl for low EOS (FIG. 9A)). These results show that SAR440340 treatment resulted in an about 24% reduction in the probability of first AECOPD at a specific time point for the low eosinophil level subgroup and an about 11% reduction in the probability of first AECOPD at a specific time point for the high eosinophil level subgroup.

### Pre-Bronchodilator (BD) FEV1

Another secondary efficacy endpoint used in the study was Pre-BD FEV1. The average change from baseline to week 16-24 in pre-bronchodilator FEV1 was analyzed using a mixed-effect model with repeated measures (MMRM) approach. Model-based averages across weeks 16, 20 and 24 were compared between the treatment groups. The dependent variable was the change from baseline in pre-bronchodilator FEV1 at each of the time points. The model included baseline FEV1 value, treatment group, visit, and treatment-by-visit interaction, the baseline eosinophil strata, and region (pooled countries), as covariates. An unstructured correlation matrix was used to model the within-patient correlations. Parameters were estimated using restricted maximum likelihood method with the Newton-Raphson algorithm. Additional covariates such as the background medications, age, height, gender, race and smoking status were considered for inclusion in the analysis model based on evaluation of blinded data and the final analysis model documented in the statistical analysis plan (SAP). Comparison between the treatment group and the placebo group were made within this model and the least square mean difference and their 95% confidence intervals was estimated. In the case of premature discontinuation of study drug the primary analysis include data up to 14 days after the last dose.

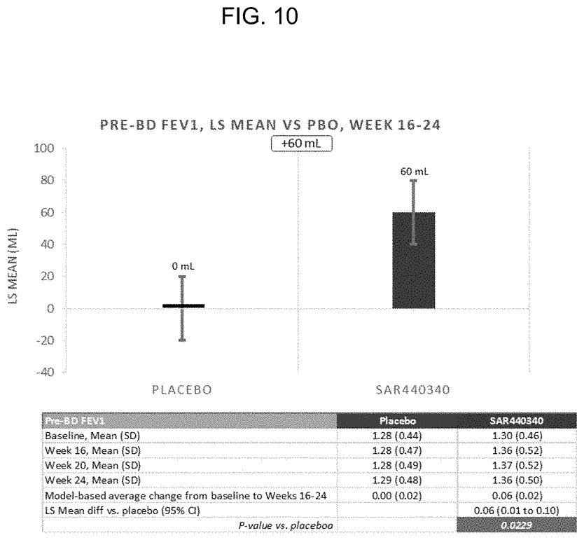

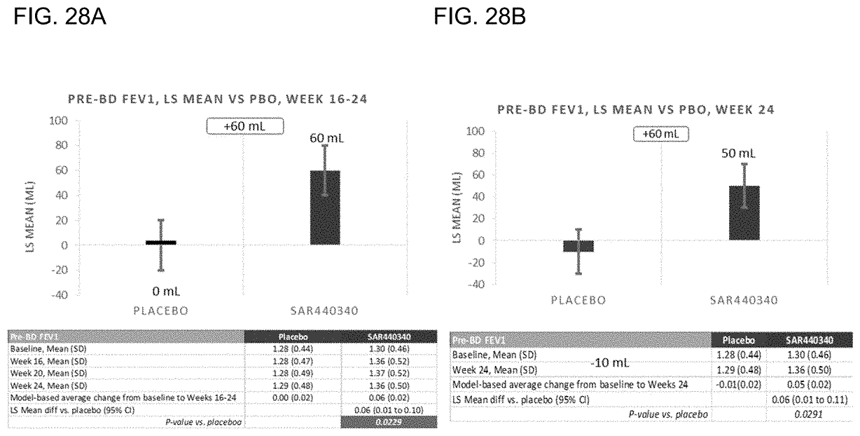

FIG. 10 shows pre-BD FEV1 change from baseline in a combined group of high and low eosinophil subjects either treated with SAR440340 or treated with placebo. The results are presented as an average change from baseline to weeks 16-24. FIG. 28 presents similar data for the average change from baseline to week 24.

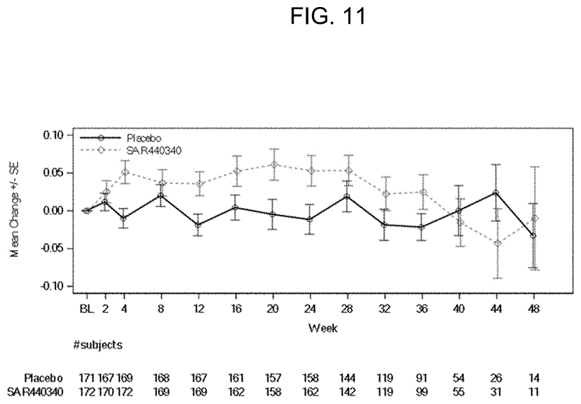

FIG. 11 shows a graph of pre-BD FEV1 mean change from baseline to week 16-24. These results show that SAR440340 treatment had a rapid and sustained effect on pre-BD FEV1.

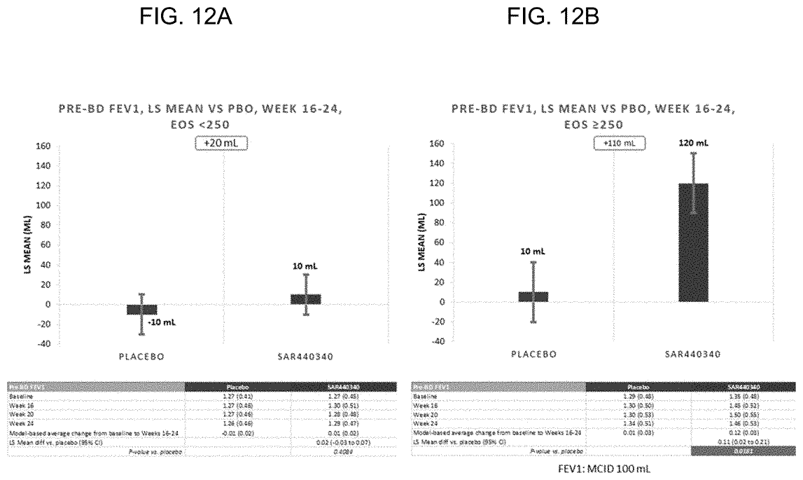

As shown in FIG. 12, subgroup analysis was performed separately by the baseline eosinophil levels (≥250/μl for high EOS (FIG. 12B) versus <250/μl for low EOS (FIG. 12A)).

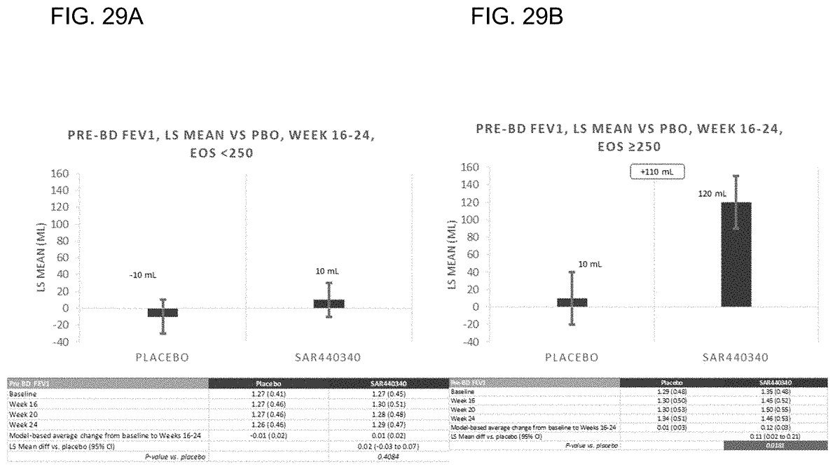

FIG. 29 presents this same data in the modified intent-to-treat analysis. The modified intent-to-treat analysis was performed as described above. These results show that SAR440340 improved pre-BD FEV1 by 110 mL in the high eosinophil level subgroup versus 20 mL in the low eosinophil level subgroup.

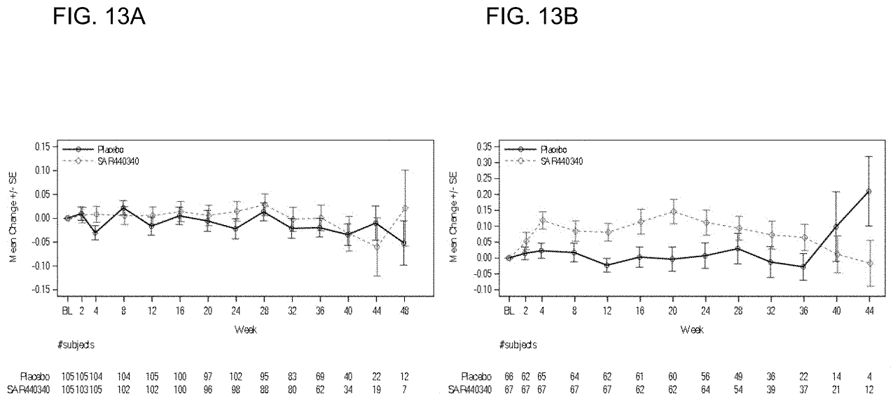

FIG. 13 presents graphs of pre-BD FEV1 mean change from baseline to week 16-24 for both the high eosinophil level group (FIG. 13B) and the low eosinophil level group (FIG. 13A). These results show that SAR440340 treatment led to a rapid and sustained improvement in lung function in the high eosinophil level subgroup.

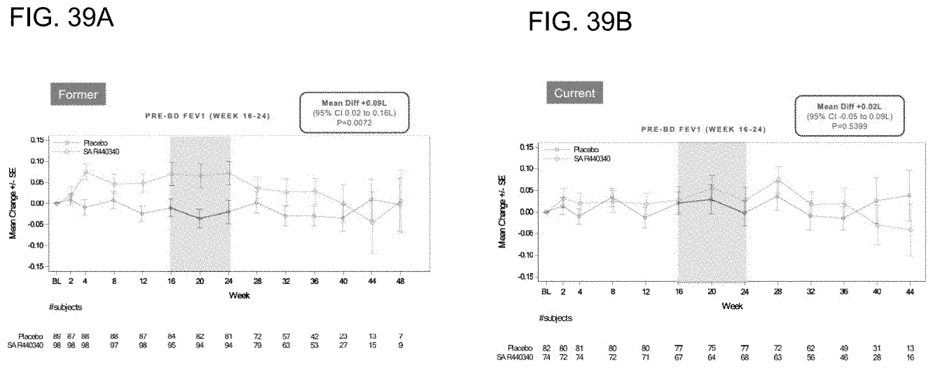

FIG. 39 presents graphs of pre-BD FEV1 mean change from baseline to week 16-24 for both the former smoker group (FIG. 39A) and the current smoker group (FIG. 39B). These results show that among former smokers, SAR440340 treatment improved pre-BD FEV1 by 90 mL. In contrast, there was no improvement in pre-BD FEV1 in current smokers.

### Post-Bronchodilator (BD) FEV1

Another secondary efficacy endpoint used in the study was post-BD FEV1. Statistical analysis for change from baseline to week 24 in FEV1 post-bronchodilator was analyzed in the same way as FEV1 pre-bronchodilator. A similar analytic method was applied to analyze change from baseline to time points past week 24 in FEV1 (both pre-bronchodilator and post-bronchodilator).

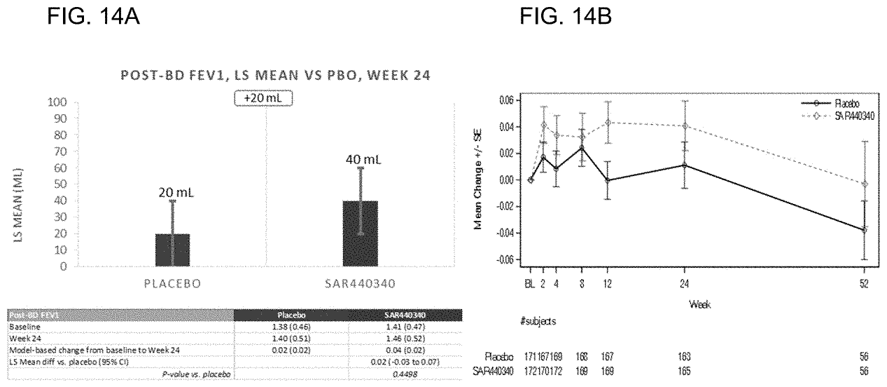

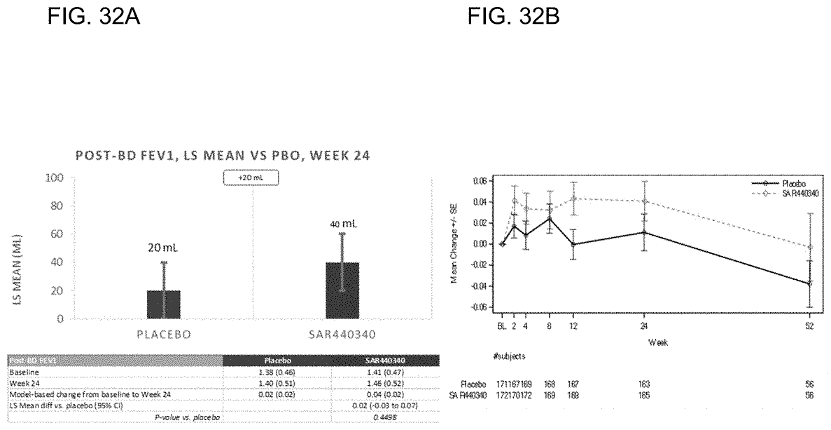

FIG. 14 shows pre-BD FEV1 change from baseline in a combined group of high and low eosinophil subjects either treated with SAR440340 or treated with placebo. FIG. 32 presents this same data in the modified intent-to-treat analysis. The modified intent-to-treat analysis was performed as described above. These results show there was a modest effect on post-BD FEV1 in the SAR440340 group.

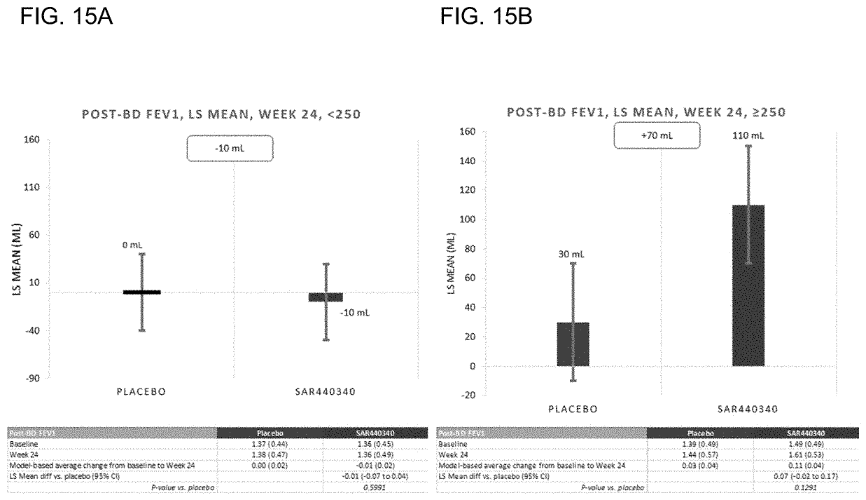

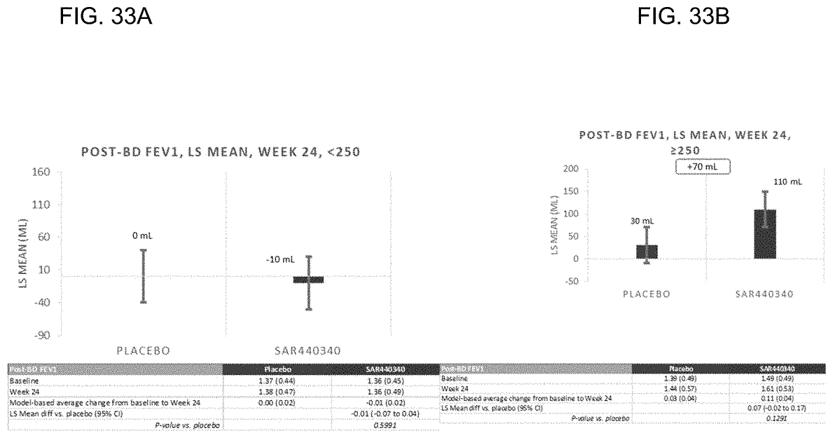

As shown in FIG. 15, subgroup analysis was performed separately by the baseline eosinophil levels (≥250/μl for high EOS (FIG. 15B) versus <250/μl for low EOS (FIG. 15A)). FIG. 33 presents this same data in the modified intent-to-treat analysis. The modified intent-to-treat analysis was performed as described above. These results show a 70 mL improvement in post-BD FEV1 in the high eosinophil level subgroup.

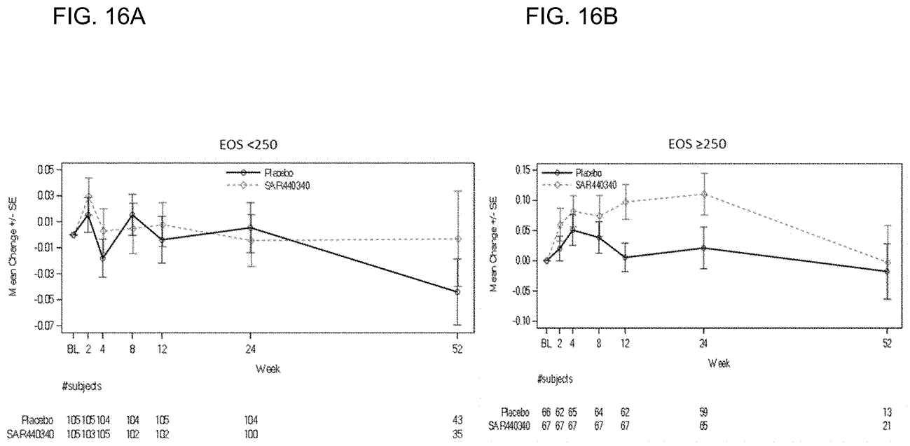

FIG. 16 presents graphs of post-BD FEV1 mean change from baseline to week 16-24 for both the high eosinophil level group and the low eosinophil level group. These results show that SAR440340 treatment showed a trend towards early and sustained improvement of post-BD FEV1 in the high eosinophil level subgroup.

### Efficacy in Smokers: Current and Former Smoker Subgroups

Table 8 and Table 9 show the baseline characteristics of former and current smoker subgroups. The baseline characteristics were balanced with the exception of FeNO levels presenting as lower in current smokers. Additionally, there was a slightly higher number of patients with baseline eosinophils ≥250, slightly fewer patients on LABA+LAMA, and slightly more patients on an ICS containing regimen in the former smoker subgroup.

TABLE 8
Baseline Characteristics, Former vs. Current Smokers.

| | N = 187 | | N = 156 | |
|---|---|---|---|---|
| Category | Former | | Current | |
| Age, mean (years) | 65.5 | (6.1) | 61.9 | (6.8) |
| Age at diagnosis of COPD (years) | 56.6 | (8.0) | 53.9 | (7.5) |
| Female (n %) | 82 | (43.9) | 67 | (42.9) |
| Time since diagnosis of COPD at randomization (years) | 9.66 | (5.90) | 8.69 | (5.57) |
| Pack years (years) | 43.96 | (26.26) | 45.52 | (20.26) |
| Time since cessation (years) | 11.80 | (9.47) | N/A | |
| Number of moderate or severe COPD exacerbations experienced within 1 year before screening visit | 2.2 | (0.9) | 2.1 | (0.9) |
| Number of moderate COPD exacerbations experienced within 1 year before screening visit | 152 | (81.2) | 122 | (78.2) |
| Number of severe COPD exacerbations experienced within 1 year before screening visit | 56 | (30.0) | 50 | (32.0) |
| Mean baseline blood eosinophil count | 0.28 | (0.22) | 0.24 | (0.21) |
| Baseline blood eosinophil counts ≥250 (n %) | 81 | (43.3) | 52 | (33.3) |
| Baseline post-BD FEV1/FVC ratio (%) | 45.54 | (10.19) | 46.21 | (10.13) |
| Baseline pre-BD FEV1 (L) | 1.27 | (0.46) | 1.31 | (0.43) |
| Baseline pre-BD FEV1 percent predicted (%) | 46.33 | (12.92) | 44.83 | (11.04) |
| Baseline post-BD FEV1 (L) | 1.36 | (0.47) | 1.43 | (0.45) |
| Baseline post-BD FEV1 percent predicted (%) | 49.69 | (12.96) | 48.88 | (11.35) |
| Baseline CAT score | 20.95 | (5.23) | 22.77 | (5.76) |
| Baseline SGRQ total score | 53.00 | (16.62) | 55.77 | (16.11) |
| ICS + LABA + LAMA (n %) | 123 | (65.8) | 103 | (66.0) |
| Baseline pre-BD FeNO (ppb) | 21.7 | (14.2) | 12.7 | (8.8) |
| Baseline post-BD FeNO (ppb) | 20.9 | (11.3) | 13.4 | (10.4) |

| | Former smokers | | | | | |
|---|---|---|---|---|---|---|
| | Placebo | | SAR440340 | | Total | |
| | (N = 89) | | 300 mg q2w(N = 98) | | (N = 187) | |
| Age, mean (years), mean(SD) | 65.4 | (6.3) | 65.6 | (6.0) | 65.5 | (6.1) |
| Age at diagnosis of COPD (years), mean(SD) | 56.4 | (8.6 | 56.9 | (7.4) | 56.6 | (8.0) |
| Female (n %) | 45 | (50.6) | 37 | (37.8) | 82 | (43.9) |
| Ethnicity [n (%)], Hispanic/Latino | 22 | (24.7) | 25 | (25.5) | 47 | (25.1) |
| Time since diagnosis of COPD at randomization | 9.83 | (6.50) | 9.51 | (5.33) | 9.66 | (5.90) |
| (years), mean (SD) | | | | | | |
| Pack years (years), mean(SD) | 42.42 | (24.56) | 45.35 | (27.76) | 43.96 | (26.26) |
| Time sincw cessation (years), mean(SD) | 12.67 | (9.57) | 11.01 | (9.35) | 11.80 | (9.47) |
| Number of moderate or severs COPD | 2.3 | (1.0) | 2.2 | (0.8) | 2.2 | (0.9) |
| exacerbations experienced within 1 year before | | | | | | |
| screening visit, mean(SD) | | | | | | |
| Number of severe COPD exacerbations | 0.3 | (0.6) | 0.4 | (0.6) | 0.28 | (0.22) |
| experienced within 1 year before screening visit, | | | | | | |
| mean(SD) | | | | | | |
| Mean baseline blood eosinophil count, mean(SD) | 0.27 | (0.21) | 0.28 | (0.23) | 0.28 | (0.22) |
| Baseline EOS (≥250), n(%) | 37 | (41.6) | 44 | (44.9) | 81 | (43.3) |
| Baseline post-BD FEV1/FVC ratio (%), mean(SD) | 46.94 | (11.36) | 44.27 | (8.87) | 45.54 | (10.19) |
| Baseline pre-BD FEV1 (L), mean(SD) | 1.23 | (0.43) | 1.32 | (0.49) | 1.27 | (0.46) |
| Baseline pre-BD FEV1 percent predicted (%), | 46.03 | (13.15) | 46.61 | (12.77) | 46.33 | (12.92) |
| mean(SD) | | | | | | |
| Baseline post-BD FEV1 (L), mean(SD) | 1.30 | (0.42) | 1.42 | (0.50) | 1.36 | (0.47) |
| Baseline Post-BD FEV1 percent predicted (%), | 48.87 | (13.15) | 50.44 | (12.90) | 49.69 | (12.96) |
| mean(SD) | | | | | | |
| Baseline FEV1 reversibility (%), mean(SD) | 7.08 | (9.69) | 9.26 | (11.30) | 8.22 | (10.59) |
| Baseline SGRQ total score, mean(SD) | 54.11 | (17.24) | 51.98 | (16.07) | 53.00 | (16.62) |
| Baseline CAT score, Mean (SD) | 21.39 | (5.05) | 20.54 | (5.37) | 20.95 | (5.23) |
| ICS + LABA + LAMA, n(%) | 59 | (66.3) | 64 | (65.3) | 123 | (65.8) |
| LABA + LAMA, n(%) | 11 | (12.4) | 12 | (12.2) | 23 | (12.3) |
| ICS + LABA, n(%) | 19 | (21.3) | 21 | (21.4) | 40 | (21.4) |

| | | | | | |
|---|---|---|---|---|---|
| ICS + LAMA, n(%) | 0 | 1 | (1.0) | 1 | (0.5) |

| | | | | | | |
|---|---|---|---|---|---|---|
| Background therapy containing ICS [n(%)] | 77 | (86.5) | 86 | (87.8) | 163 | (87.2) |

| | Current smokers | | | | | |
|---|---|---|---|---|---|---|
| | Placebo | | SAR440340 | | Total | |
| | (N = 82) | | 300 mg q2w(N = 74) | | (N = 156) | |
| Age, mean (years), mean(SD) | 62.5 | (6.4) | 61.3 | (7.1) | 61.3 | (6.8) |
| Age at diagnosis of COPD (years), mean(SD) | 54.5 | (7.2) | 53.4 | (7.9) | 53.9 | (7.5) |
| Female (n %) | 31 | (37.8) | 36 | (48.6) | 67 | (42.9) |
| Ethnicity [n (%)], Hispanic/Latino | 11 | (13.4) | 12 | (16.2) | 23 | (14.8) |
| Time since diagnosis of COPD at randomization | 8.75 | (5.73) | 8.63 | (5.44) | 8.69 | (5.77) |
| (years), mean (SD) | | | | | | |
| Pack years (years), mean(SD) | 45.67 | (23.47) | 45.35 | (16.13) | 45.52 | (20.26) |
| Time sincw cessation (years), mean(SD) | n/a | | n/a | | n/a | |
| Number of moderate or severs COPD | 2.1 | (0.9) | 2.2 | (0.9) | 2.1 | (0.9) |
| exacerbations experienced within 1 year before | | | | | | |
| screening visit, mean(SD) | | | | | | |
| Number of severe COPD exacerbations | 0.4 | (0.6) | 0.3 | (0.6) | 0.24 | (0.21) |
| experienced within 1 year before screening visit, | | | | | | |
| mean(SD) | | | | | | |
| Mean baseline blood eosinophil count, mean(SD) | 0.23 | (0.17) | 0.26 | (0.25) | 0.24 | (0.21) |
| Baseline EOS (≥250), n(%) | 29 | (35.4) | 23 | (31.1) | 52 | (33.3) |
| Baseline post-BD FEV1/FVC ratio (%), mean(SD) | 45.58 | (9.87) | 46.90 | (10.44) | 46.21 | (10.13) |
| Baseline pre-BD FEV1 (L), mean(SD) | 1.34 | (0.44) | 1.28 | (0.42) | 1.31 | (0.43) |
| Baseline pre-BD FEV1 percent predicted (%), | 45.16 | (10.95) | 44.46 | (11.20) | 44.83 | (11.04) |
| mean(SD) | | | | | | |
| Baseline post-BD FEV1 (L), mean(SD) | 1.46 | (0.48) | 1.40 | (0.43) | 1.43 | (0.45) |
| Baseline Post-BD FEV1 percent predicted (%), | 49.19 | (11.23) | 48.54 | (11.55) | 48.88 | (11.35) |
| mean(SD) | | | | | | |
| Baseline FEV1 reversibility (%), mean(SD) | 9.60 | (9.67) | 10.07 | (10.62) | 9.83 | (10.10) |
| Baseline SGRQ total score, mean(SD) | 54.03 | (15.31) | 57.69 | (16.85) | 55.77 | (16.11) |
| Baseline CAT score, Mean (SD) | 21.95 | (5.44) | 23.68 | (6.00) | 22.77 | (5.76) |
| ICS + LABA + LAMA, n(%) | 52 | (63.4) | 51 | (68.9) | 103 | (66.0) |
| LABA + LAMA, n(%) | 15 | (18.3) | 11 | (14.9) | 26 | (16.7) |
| ICS + LABA, n(%) | 15 | (18.3) | 12 | (16.2) | 27 | (17.3) |
| ICS + LAMA, n(%) | 0 | | 0 | | 0 | |
| Background therapy containing ICS [n(%)] | 68 | (82.9) | 63 | (85.1) | 131 | (84.0) |

TABLE 9
Baseline Characteristics Within Smoker Subgroups.

| | | Former | | Current | |
|---|---|---|---|---|---|
| | Sta- | Placebo | SAR440340 | Placebo | SAR440340 |
| Category | tistics | (N = 89) | (N = 98) | (N = 82) | (N = 74) |
| Age mean | Mean | 65.4 | 55.6 | 62.5 | 61.3 |
| (Years) | (SD) | (6.3) | (6.0) | (6.4) | (7.1) |
| Age at | Mean | 56.4 | 58.9 | 54.5 | 53.4 |
| diagnosis of | | | | | |
| COPD (years) | (SD) | (8.6) | (7.4) | (7.2) | (7.9) |
| Female (n %) | n (%) | 45 | 37 | 31 | 36 |
| | | (50.6) | (37.8) | (37.8) | (48.6) |
| Ethnicity | n (%) | 22 | 25 | 11 | 12 |
| [n (%)], | | | | | |
| Hispanic/Latino | | (24.7) | (25.9) | (13.4) | (16.2) |
| Time since | Mean | 9.83 | 9.51 | 5.75 | 8.63 |
| diagnosis | | | | | |
| of COPD at | (SD) | (6.50) | (5.33) | (5.73) | (5.44) |
| randomization | | | | | |
| (years) | | | | | |
| Pack years | Mean | 42.42 | 45.35 | 45.67 | 45.35 |
| (years) | (SD) | (24.56) | (27.76) | (23.47) | (16.13) |
| Time since | Mean | 12.67 | 11.01 | n/a | n/a |
| cessation | (SD) | (9.57) | (9.35) | | |
| (years) | | | | | |
| Number of | Mean | 2.3 | 2.2 | 2.1 | 2.2 |
| moderate to | (SD) | (1.0) | (0.8) | (0.9) | (0.9) |
| severe COPD | | | | | |
| exacerbations | | | | | |
| experienced | | | | | |
| within 1 | | | | | |
| year, before | | | | | |
| screening visit | | | | | |
| Number of | Mean | 2.0 | 1.8 | 1.6 | 1.9 |
| moderate | (SD) | (1.2) | (1.1) | (1.1) | (0.6) |
| COPD | | | | | |
| exacerbations | | | | | |
| experienced | | | | | |
| within 1 | | | | | |
| year before | | | | | |
| screening visit | | | | | |
| Number of | Mean | 0.3 | 0.4 | 0.4 | 0.3 |
| severe COPD | (SD) | (0.6) | (0.6) | (0.6) | (0.6) |
| exacerbations | | | | | |
| experienced | | | | | |
| 1 year before | | | | | |
| screening | | | | | |
| Mean baseline | Mean | 0.27 | 0.28 | 0.23 | 9.26 |
| blood | (SD) | (0.21) | (0.33) | (0.17) | (0.25) |
| eosinophil | | | | | |
| count | | | | | |
| Mean baseline | n (%) | 37 | 44 | 29 | 23 |
| blood | | (41.6) | (44.9) | (35.4) | (31.1) |
| eosinophil | | | | | |
| count (≥250) | | | | | |
| Baseline | Mean | 46.94 | 44.7 | 45.58 | 46.90 |
| post-BD | (SD) | (11.35) | (6.87) | (9.87) | (10.44) |
| FEV1/FVO | | | | | |
| ratio (%) | | | | | |
| Baseline | Mean | 1.23 | 1.32 | 1.34 | 1.28 |
| pre-BD | (SD) | (0.43) | (0.49) | (0.44) | (0.42) |
| FEV1 (L) | | | | | |
| Baseline | Mean | 46.03 | 46.61 | 45.16 | 44.46 |
| pre-BD | (SD) | (13.15) | (12.77) | (10.95) | (11.20) |
| FEV1 percent | | | | | |
| predicted (%) | | | | | |
| Baseline | Mean | 1.30 | 1.42 | 1.46 | 1.40 |
| post-BD | (SD) | (0.42) | (0.50) | (0.48) | (0.43) |
| FEV1 (L) | | | | | |
| Baseline | Mean | 48.87 | 50.44 | 49.19 | 48.54 |
| post-BD | (SD) | (13.06) | (12.90) | (11.23) | (11.55) |
| FEV1 percent | | | | | |
| predicted (%) | | | | | |
| Baseline | Mean | 7.08 | 9.25 | 9.60 | 10.07 |
| FEV1 | (SD) | (9.69) | (11.30) | (9.67) | (10.52) |
| reversibility | | | | | |
| (%) | | | | | |
| Baseline | Mean | 54.11 | 51.98 | 54.03 | 57.69 |
| SGRQ | (SD) | (17.24) | (16.07) | (15.31) | (16.85) |
| total score | | | | | |
| ICS + LABA + | n (%) | 59 | 64 | 52 | 51 |
| LAMA | | (66.3) | (65.3) | (63.4) | (66.9) |
| Baseline | Mean | 20.9 | 22.8 | 11.0 | 15.5 |
| pre-BD | (SD) | (12.1) | (17.7) | (7.0) | (11.3) |
| FeNO (ppb) | | | | | |
| Baseline | Mean | 23.4 | 17.6 | 12.2 | 15.4 |
| post-BD | (SD) | (12.2) | (10.0) | (7.9) | (14.1) |
| FeNO (ppb) | | | | | |
| Baseline total | Mean | 303.12 | 252.51 | 339.21 | 453.36 |
| IgE (kU/L) | (SD) | (824.11) | (441.20) | (669.59) | (1037.59) |

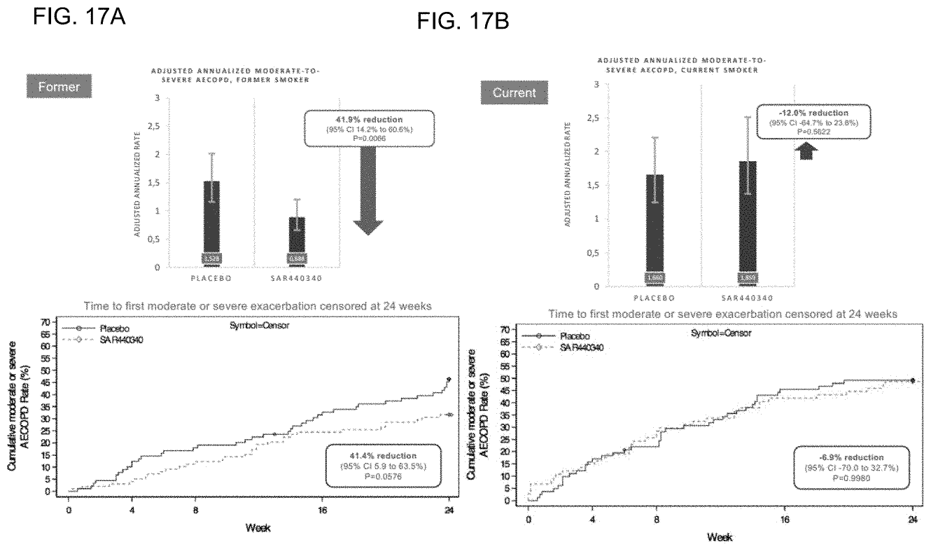

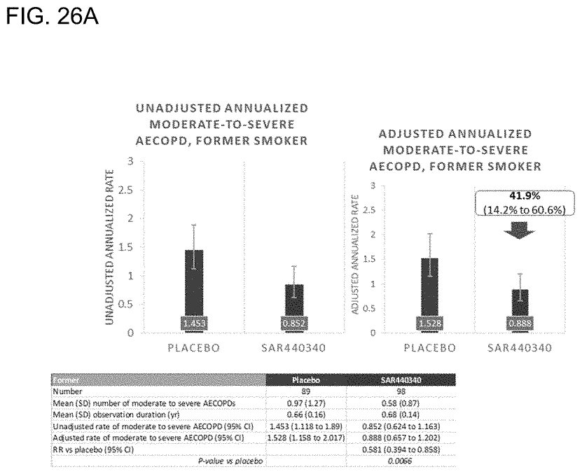

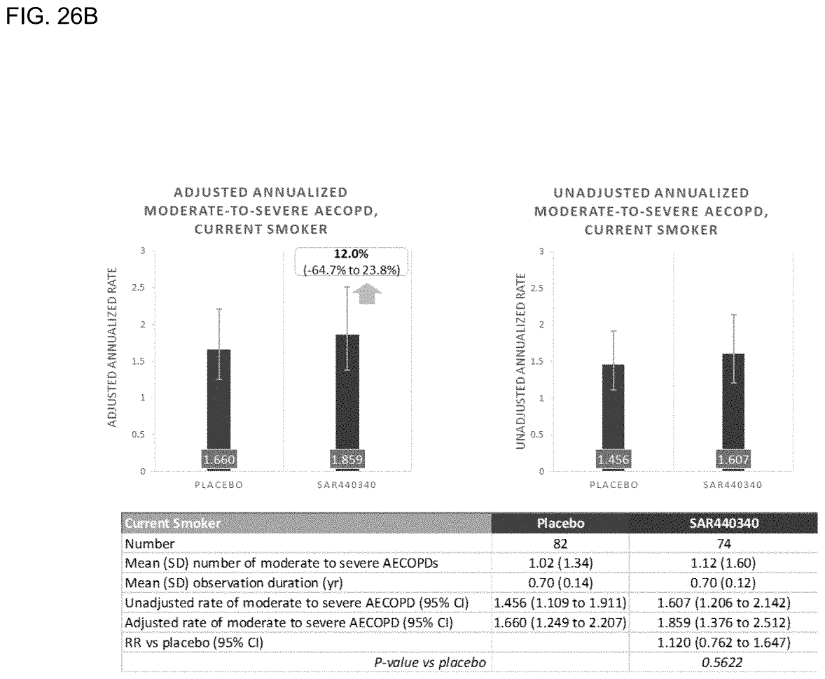

FIG. 17 shows annualized and cumulative moderate-to-severe AECOPD in a combined group of high and low eosinophil subjects either treated with SAR440340 or treated with placebo. Data is presented for both current (FIG. 17B) and former smokers (FIG. 17A) as subgroups. In the former smoker subgroup, SAR440340 reduced annualized moderate-to-severe COPD exacerbations by 42% and improved pre-BD FEV1190 mL. In contrast, there was an increase of 120 in the current smoker group, and no improvement in FEV1. FIG. 26A shows the unadjusted annualized moderate-to-severe AECOPD in comparison to the adjusted annualized moderate-to-severe AECOPD for the former smoker subgroup. FIG. 26B shows the unadjusted annualized moderate-to-severe AECOPD in comparison to the adjusted annualized moderate-to-severe AECOPD for the current smoker subgroup. The adjusted and unadjusted values for annualized moderate-to-severe AECOPD were calculated as described above. This data shows that SAR440340 treatment led to an about 42% reduction in AECOPD in former smokers.

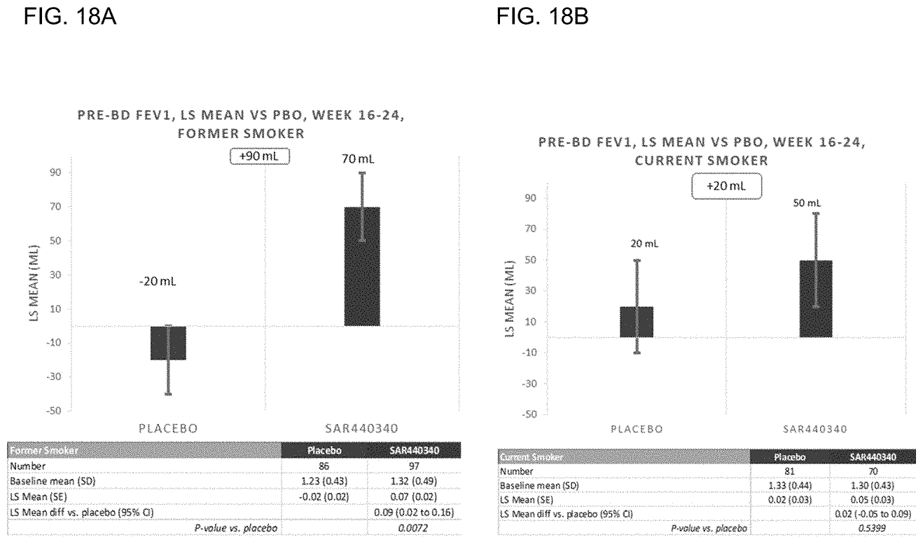

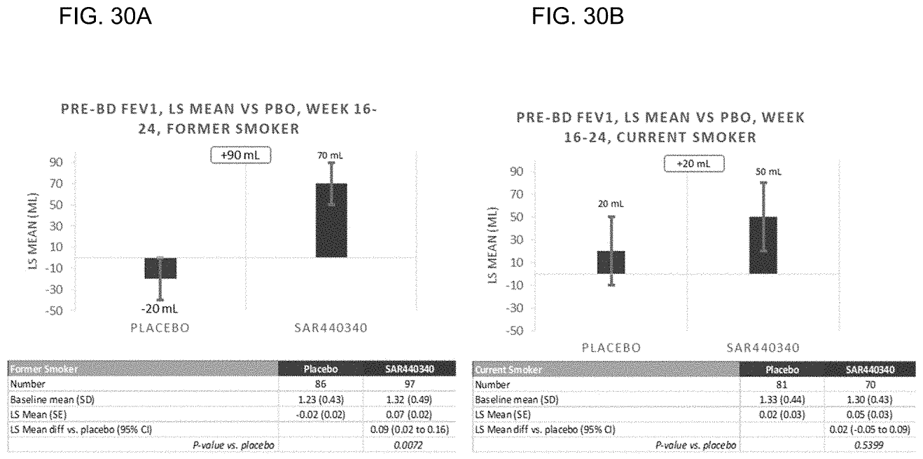

FIG. 18 shows pre-BD FEV1 change from baseline in a combined group of high and low eosinophil subjects either treated with SAR440340 or treated with placebo. Data is presented for both current (FIG. 18B) and former smokers (FIG. 18A) as subgroups. FIG. 30 presents this same data in the modified intent-to-treat analysis. The modified intent-to-treat analysis was performed as described above. These data show that SAR440340 treatment led to an about 90 mL improvement in pre-BD FEV1 in former smokers and an about 20 mL improvement in pre-BD FEV1 in current smokers.

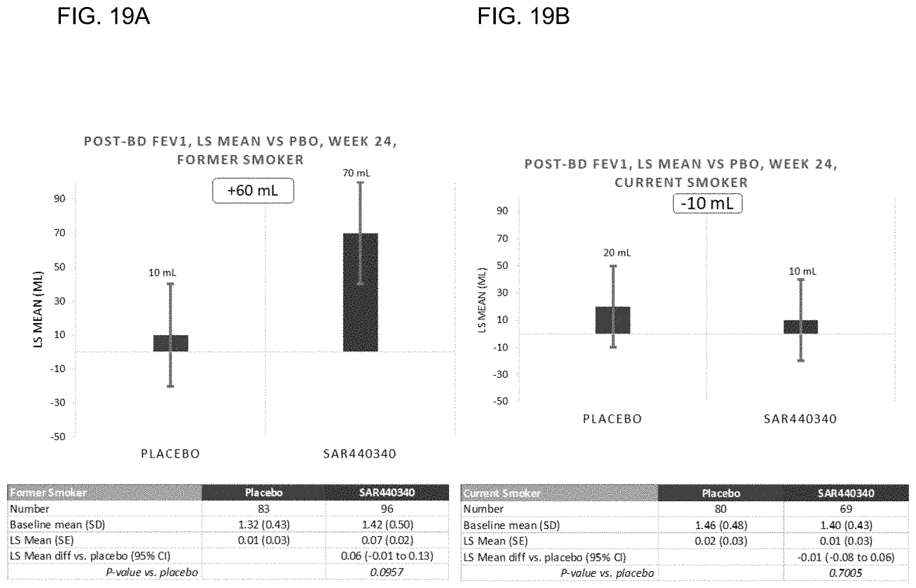

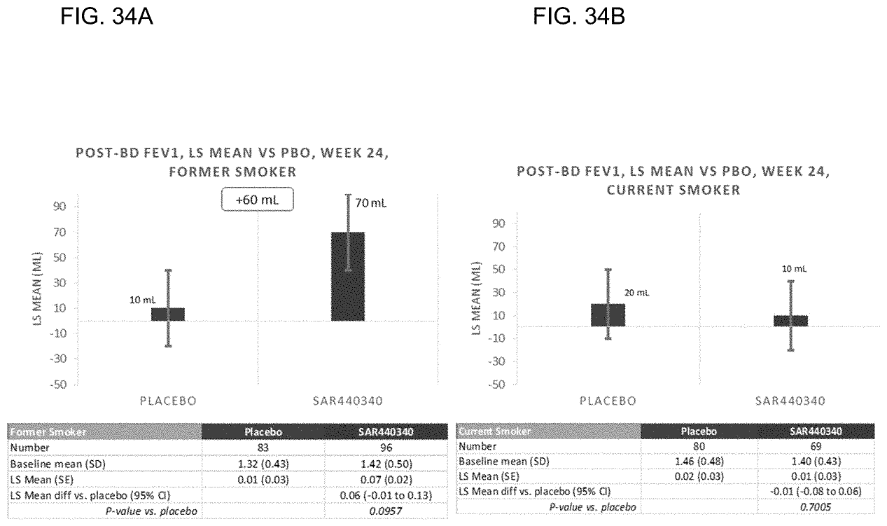

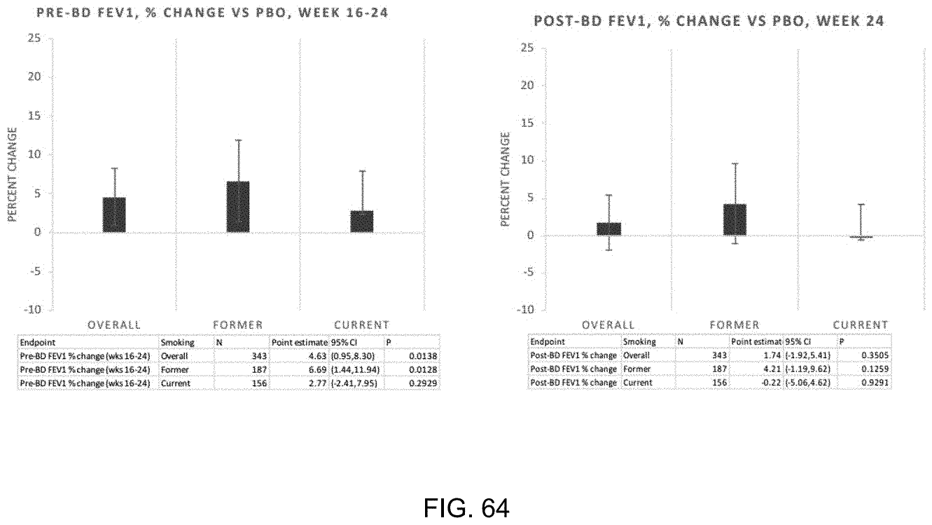

FIG. 19 shows post-BD FEV1 change from baseline in a combined group of high and low eosinophil subjects either treated with SAR440340 or treated with placebo. Data is presented for both current (FIG. 19B) and former (FIG. 19A) smokers as subgroups. FIG. 34 presents this same data in the modified intent-to-treat analysis. The modified intent-to-treat analysis was performed as described above. These data show that SAR440340 treatment led to an about 60 mL improvement in post-BD FEV1 in former smokers. Former smokers had the most improvement in percent change in FEV1 both pre-BD and post-BD (FIG. 64).

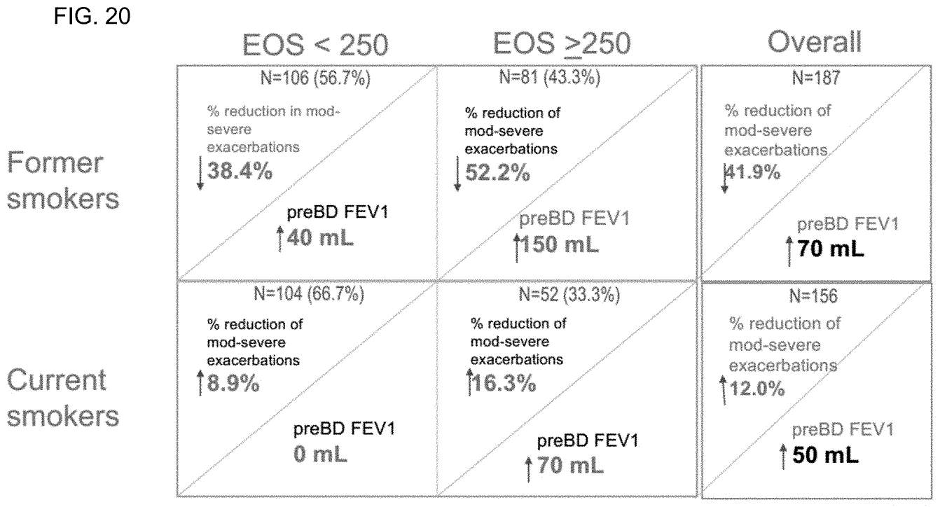

FIG. 20 presents data related to the efficacy outcome relationship to both smoking status and eosinophil subgroup. These data show that the highest efficacy in preventing AECOPD was observed in former smokers treated with SAR440340 regardless of eosinophil subgroup.

The annualized rate of moderate-to-severe AECOPD events (primary endpoint) was 1.61 in the placebo group and 1.30 in the itepekimab group (relative risk [RR] 0.81; 95% CI [confidence interval] 0.61 to 1.07), while the LS mean change from baseline to weeks 16 to 24 in pre-bronchodilator FEV1 (key secondary endpoint) was 0.00 L with placebo and 0.06 L with itepekimab in the mITT population (LS mean difference 0.06; 95% CI 0.01 to 0.10). All of the benefit in both AECOPD and FEV1 could be accounted for by more pronounced treatment effects in the former smoker subgroup, with no treatment benefit in the complementarity current smokers subgroup. The overall AECOPD treatment effect was driven by a pronounced 42.5% reduction in AECOPD versus placebo in the subgroup of former smokers (RR 0.58; 95% CI 0.39 to 0.85; HR for time to first AECOPD event 0.57; 95% CI 0.37 to 0.88), compared to no effect observed in current smokers (RR 1.09; 95% CI 0.74 to 1.61; HR 1.15; 95% CI 0.75 to 1.77). Similarly, the FEV1 treatment effect was most pronounced in the subgroup in former smokers (LS mean difference 0.09; 95% CI 0.02 to 0.15) with no treatment effect in current smokers.

While treatment effect on AECOPD was not associated with eosinophil levels, treatment effect on FEV₁ was higher in the subgroup of patients with eosinophils ≥250 cells/mm³ (LS mean difference 0.12; 95% CI 0.02 to 0.21).

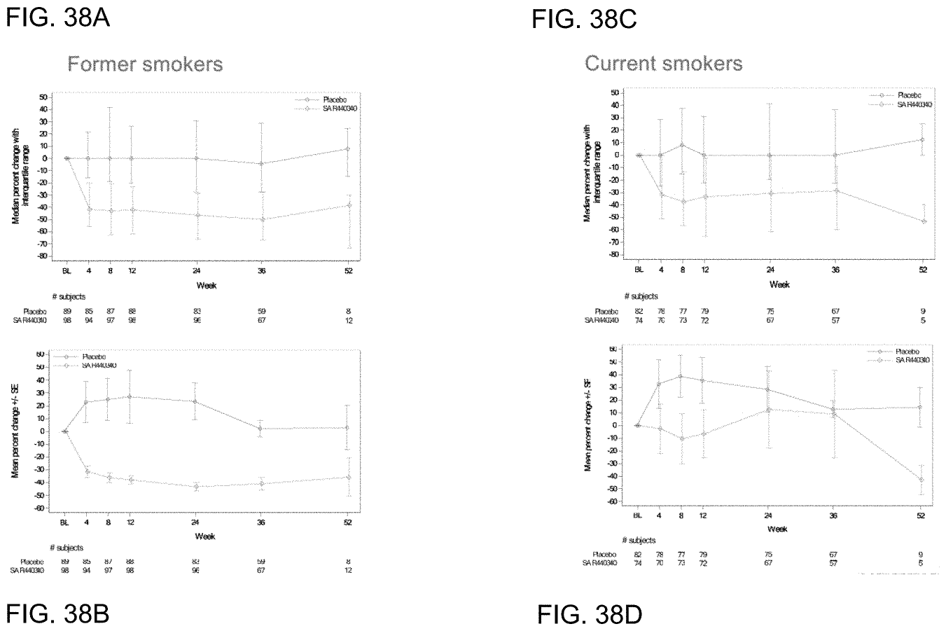

FIG. 38A-FIG. 38D graphically depict the effect of SAR440340 on blood eosinophil levels. Data is presented for median (FIG. 38A) and mean (FIG. 38B) percent change of eosinophils in former smokers and median (FIG. 38C) and mean (FIG. 38D) percent change of eosinophils in current smokers.

### Efficacy in Moderate COPD Versus Severe COPD Categories

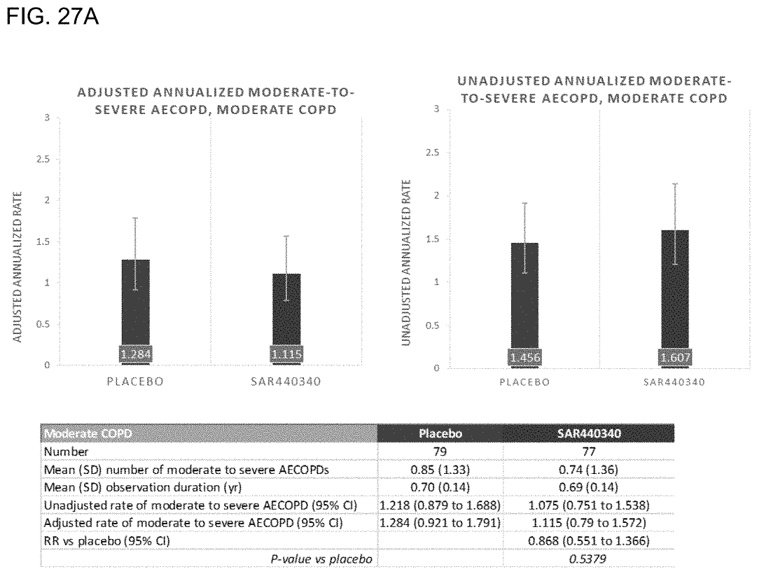

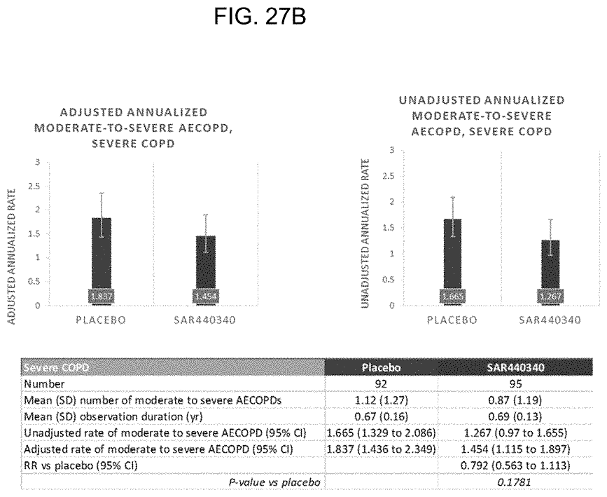

FIG. 27A shows data for both adjusted and unadjusted annualized moderate-to-severe AECOPD in participants with moderate COPD for both SAR440340 treated and placebo groups. FIG. 27B shows data for both adjusted and unadjusted annualized moderate-to-severe AECOPD in participants with severe COPD for both SAR440340 treated and placebo groups. These results show that there was no significant difference in efficacy of treatment based on COPD classification.

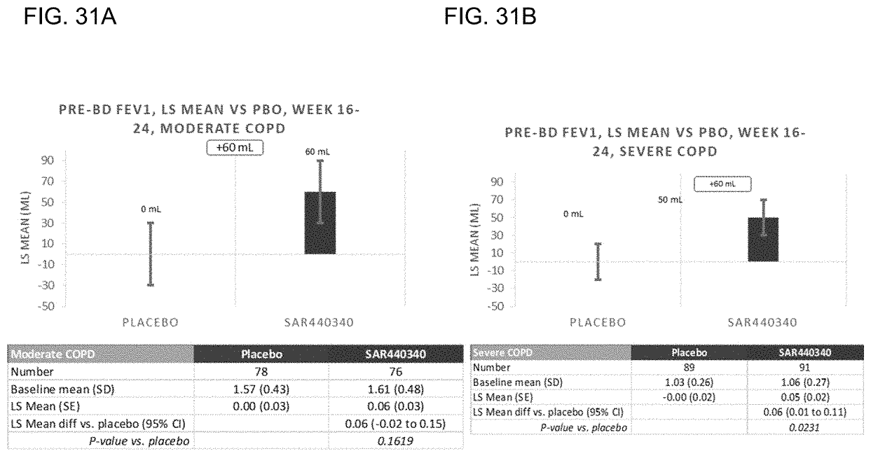

FIG. 31A shows data for pre-BD FEV1 for participants with moderate COPD treated with SAR440340 or placebo. FIG. 31B shows data for pre-BD FEV1 for participants with severe COPD treated with SAR440340 or placebo. These results show that SAR440340 led to an improvement in pre-BD FEV1 in patients with lower lung function.

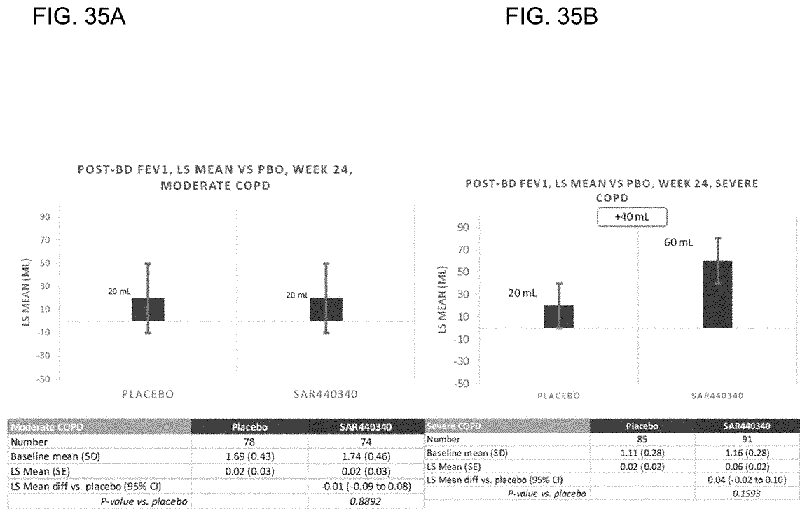

FIG. 35A shows data for post-BD FEV1 for participants with moderate COPD treated with SAR440340 or placebo. FIG. 351B shows data for post-BD FEV1 for participants with severe COPD treated with SAR440340 or placebo. These results show that SAR440340 led to an improvement in post-BD FEV1 in patients with lower lung function.

St. George's Respiratory Questionnaire (SGRQ)

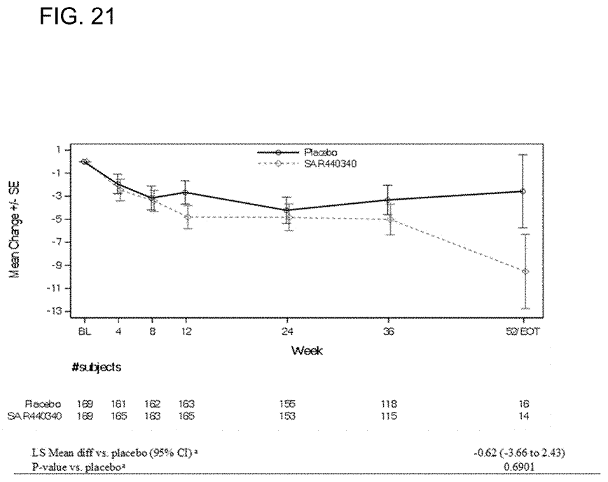

FIG. 21 shows SGRQ change from baseline in a combined group of high and low eosinophil subjects either treated with SAR440340 or treated with placebo. These results show that there was no change in SGRQ for the SAR440340 treated group.

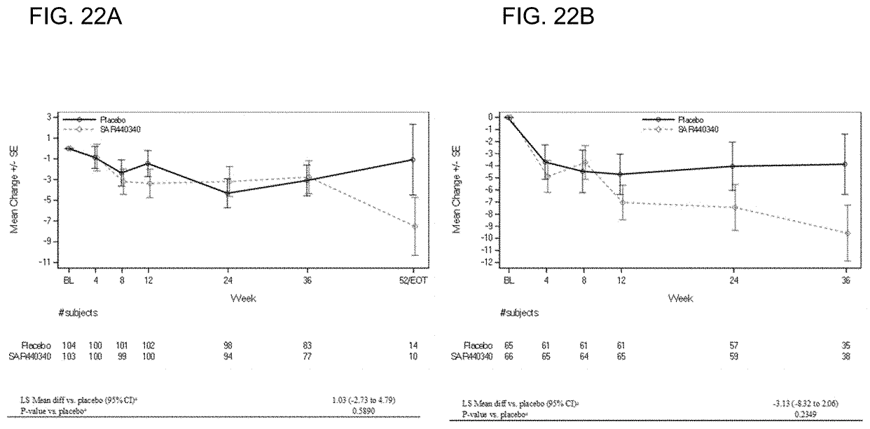

As shown in FIG. 22, subgroup analysis was performed separately by the baseline eosinophil levels (≥250/μl for high EOS (FIG. 22B) versus <250/μl for low EOS (FIG. 22A)). These results show that SAR440340 lead to an improvement in SGRQ in the high EOS subgroup.

### Biomarkers

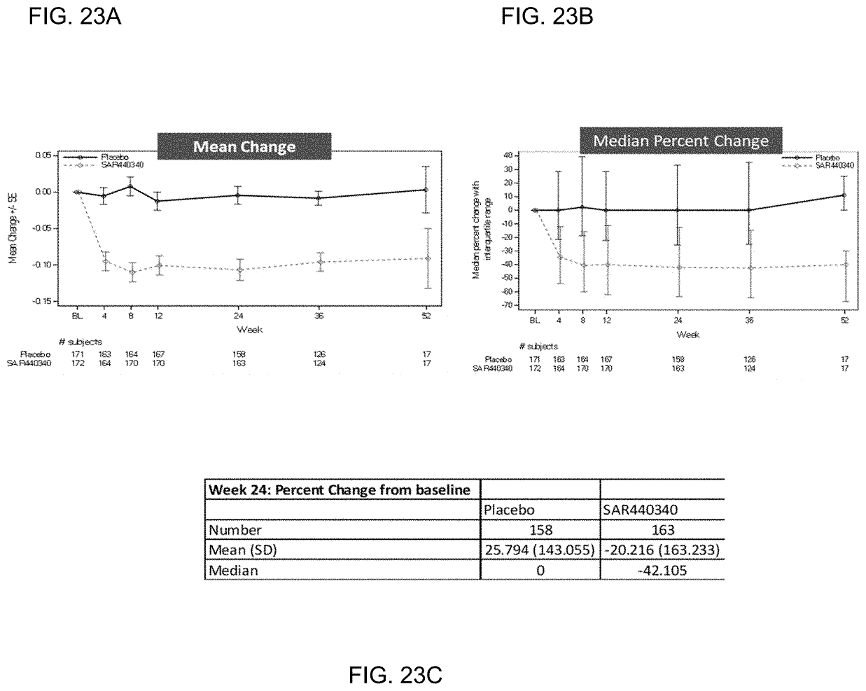

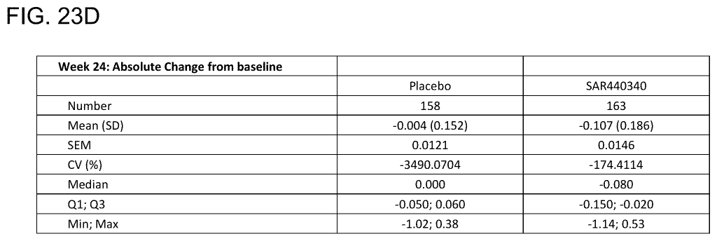

FIG. 23 shows data related to the level of blood eosinophils in subjects either treated with SAR440340 or treated with placebo. FIG. 23A shows the mean change in blood eosinophils and FIG. 23B shows the median percent change in blood eosinophils. FIG. 23C shows percent change from baseline at week 24. These data show that SAR440340 treatment led to a rapid and sustained reduction in blood eosinophils, with an about −42% median change.

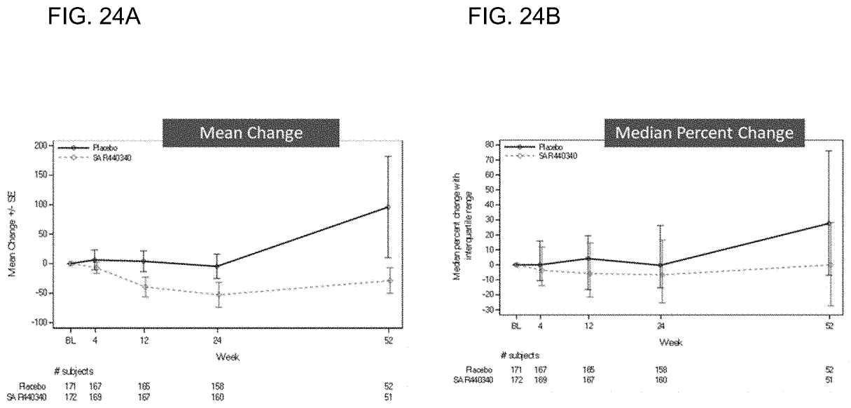

FIG. 24 shows data related to the level of the biomarker IgE in subjects either treated with SAR440340 or treated with placebo. FIG. 24A shows the mean percent change from baseline in IgE levels. FIG. 24B shows the median percent change from baseline in IgE levels. These data show that there was a slight reduction in IgE levels from baseline in the SAR440340 treatment group.

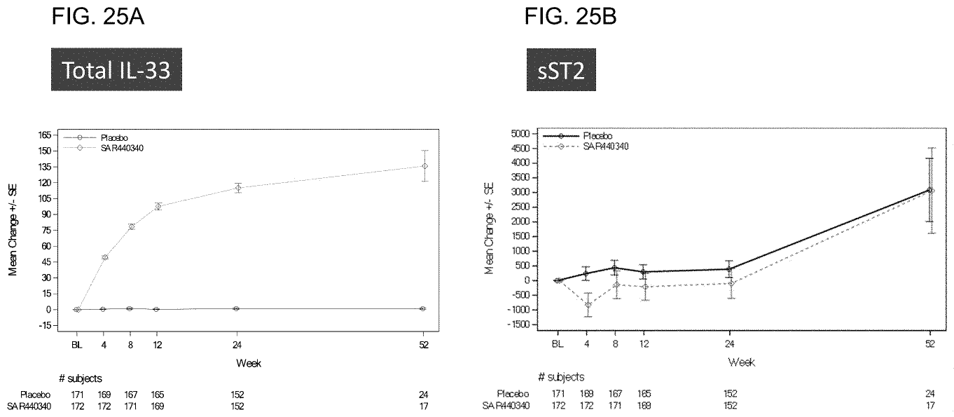

FIG. 25 shows data related to levels of total IL-33 and sST2 in subjects either treated with SAR440340 or treated with placebo. FIG. 25A shows mean change in total IL-33. FIG. 25B shows mean change in sST2. These data show that there was significant impact of SAR440340 treatment on IL-33, and not on sST2.

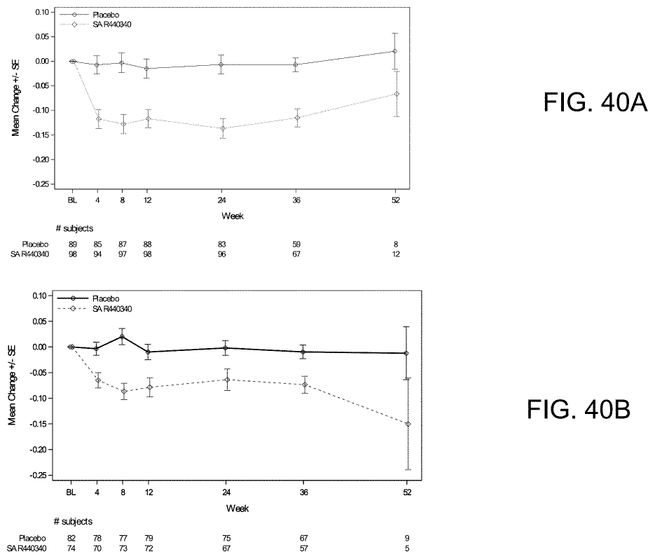

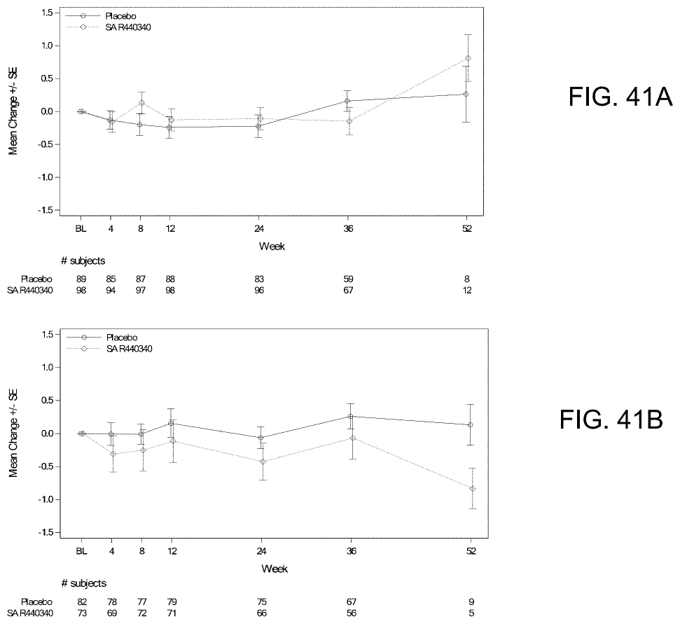

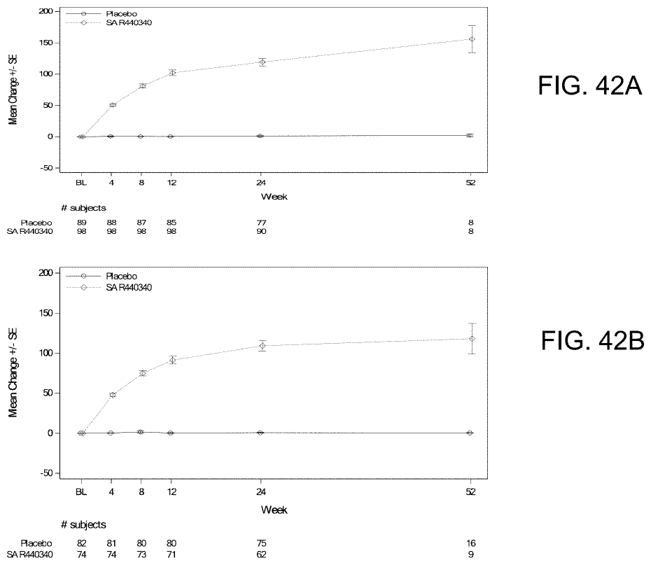

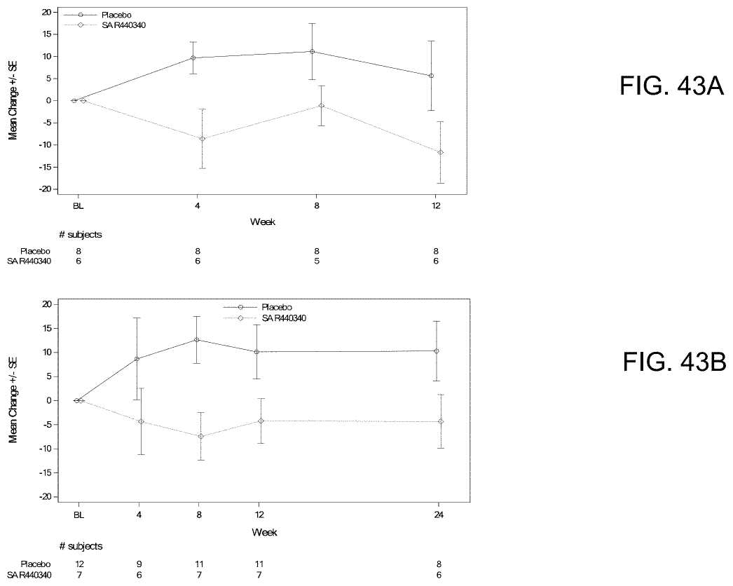

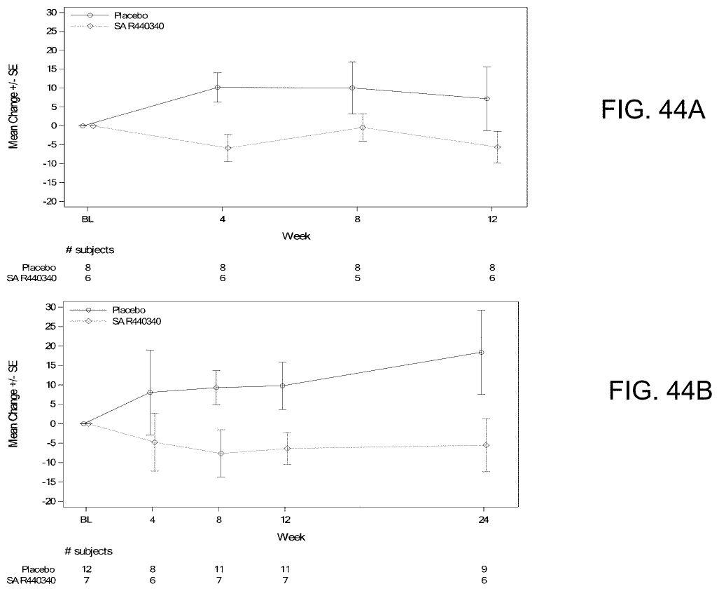

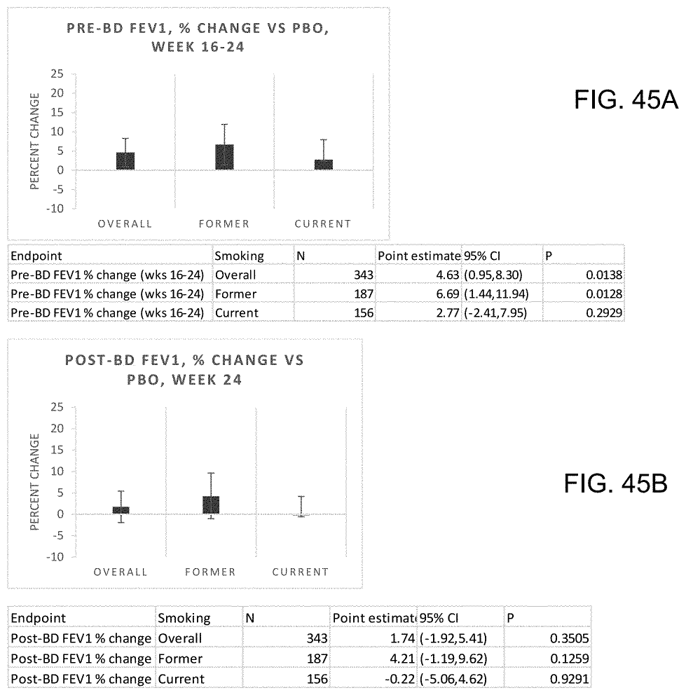

FIG. 40A-FIG. 40B depict mean change in blood eosinophils in former smokers vs. current smokers, respectively. A similar affect was observed in both groups, but a larger effect was seen in former smokers. FIG. 41A-FIG. 41B depict mean change in neutrophils in former smokers vs. current smokers, respectively. FIG. 42A-FIG. 42B depict mean change in total IL-33 in former smokers vs. current smokers, respectively. FIG. 43A-FIG. 43B depict mean change in pre-bronchodilator (pre-BD) FeNO in former smokers vs. current smokers, respectively. FIG. 44A-FIG. 44B depict mean change in post-bronchodilator (post-BD) FeNO in former smokers vs. current smokers, respectively. Former smokers had the most improvement in percent change in FEV1. (See FIG. 45A-FIG. 45B.)

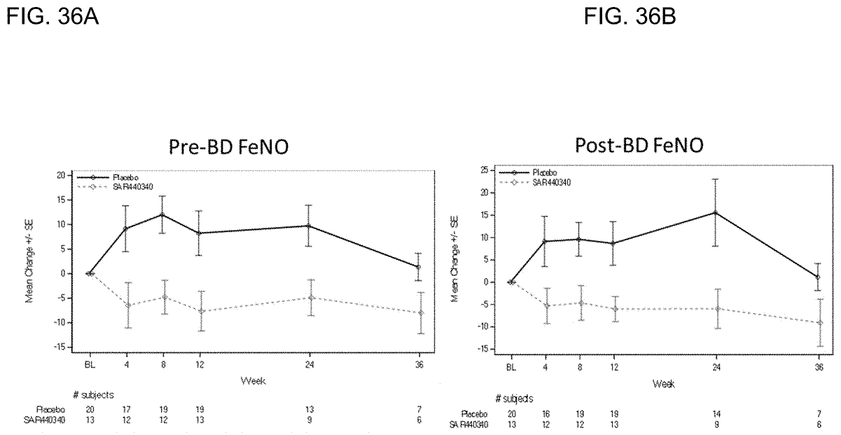

FIG. 36 depicts mean change from baseline in pre-BD and post-BD FeNO, showing there was a reduction in FeNO.

The statistical methods used to analyze the primary and secondary endpoints are shown below in Table 10.

TABLE 10
Efficacy Analyses.

| Endpoint | Statistical Analysis Methods |
|---|---|
| Primary | For the primary efficacy endpoint, the |
| Rate of moderate- | annualized exacerbation rate, a negative |
| to-severe | binomial regression model will be used to |
| AECOPD | assess treatment differences. The model |
| over 52 weeks. | will include the total number of events |
| | occurring during the observation period |
| | (up to Week 52) as response variable, |
| | and the treatment group, the baseline |
| | eosinophil strata and region (pooled |
| | countries) as covariates. Log-transformed |
| | observation duration will be the offset variable. |
| | Parameters will be estimated using the |
| | maximum likelihood method with the Newton- |
| | Raphson algorithm. |
| | The annualized event rate for each of the |
| | treatment groups and the ratio between the |
| | treatment and placebo along with its 95% |
| | confidence interval will be estimated from |
| | the model. |
| | In the case of premature discontinuation of |
| | study drug, a secondary analysis will include |
| | events up to 14 days after the last dose. |
| | Using the same method, subgroup analyses |
| | will be performed separately by the baseline |
| | eosinophil levels (≥250 /mm³ versus <250/mm³). |
| Secondary | |
| Key Secondary | Key Secondary Endpoint |
| Change from | The key secondary efficacy endpoint, the |
| baseline in | average change from baseline to Week 16-24 |
| FEV1 to | in pre-bronchodilator FEV1 will be analyzed |
| Week 16-24 | using a mixed-effect model with repeated |
| (pre- | measures (MMRM) approach. Model-based |
| bronchodilator*) | averages across Weeks 16, 20 and 24 will be |
| | compared between the treatment groups. |
| | The dependent variable is the change from |
| | baseline in pre-bronchodilator FEV1 at each |
| | time points. The model will include baseline |
| | FEV1 value, treatment group, visit, and |
| | treatment-by-visit interaction, the baseline |
| | eosinophil strata, and region (pooled countries), |
| | as covariates. An unstructured correlation matrix |
| | will be used to model the within-patient |
| | correlations. Parameters will be estimated using |
| | restricted maximum likelihood method with the |
| | Newton-Raphson algorithm. Additional covariates |
| | such as the background medications, age, height, |
| | gender, race and smoking status will be considered |
| | for inclusion in the analysis model based on |
| | evaluation of blinded data and the final analysis |
| | model documented in the statistical analysis |
| | plan (SAP). |
| | Comparison between the treatment group and |
| | the placebo group will be made within this model |
| | nd the least square mean difference and their 95% |
| | confidence intervals will be estimated. |
| | In the case of premature discontinuation of study |
| | drug the primary analysis will include data up |
| | to 14 days after the last dose. |
| | Subgroup analyses will be performed using the |
| | same method by baseline eosinophil levels |
| | (≥250/mm³ versus <250/mm³) |
| Other Secondary | Other secondary endpoints |
| Change from | Change from baseline to Week 24 in FEV1 |
| baseline in | post-bronchodilator will be analyzed in the same |
| FEV1 to | way as the key secondary efficacy endpoint. |
| Week 24 (post- | Similar analytic method will be applied to |
| bronchodilator*) | analyze change from baseline to time points |
| Time to first | past 24 in FEV1 (both pre-bronchodilator and |
| moderate or | post-bronchodilator). |
| severe AECOPD | Time to first moderate or severe AECOPD will |
| | be analyzed using a Cox regression model |
| | with treatment, baseline eosinophil strata, and |
| | region (pooled country) as covariates. The |
| | Kaplan-Meier (K-M) method will be used to |
| | estimate the probabilities of first AECOPD at |
| | specific time points for each group. Additional |
| | covariates will be considered based on |
| | evaluation of blinded data and the final analysis |
| | model documented in the SAP. |
| Exploratory | Will be described in the statistical analysis plan |
| | finalized before database lock |

### Summary of Results

As summarized in Table 11 below, SAR440340 reduced moderate-to-severe exacerbations in COPD patients by 18% (ns, p=0.1647), irrespective of participant blood eosinophil level. SAR440340 improved pre-BD FEV1 of 60 mL in the overall population (low and high eosinophils), with clear trend for higher efficacy in high EOS (110 mL) versus low EOS (20 mL), and with a rapid onset of action (4 weeks). The efficacy level observed in former smokers, for both exacerbation reduction and FEV1 improvement, was improved, with 42% (p=0.0066) and 90 mL (p=0.0072), respectively. Taken together, these data indicate that SAR440340, on top of standard of care (SOC) (double or triple therapy), can have independently a bronchodilator effect, fast onset of action (primarily in high EOS patients), and a preventive effect for exacerbations in overall population. These beneficial effects were more prominent in former smokers, indicating that the absence of continuous epithelium stress by cigarette smoke allows for a faster reparative/disease modifying effect of SAR440340.

SAR440340 numerically decreased the annualized rate of AECOPD (19% reduction) and also improved pre-bronchodilator FEV₁ (0.06 L improvement), but did not meet statistical significance. However, all of the potential benefit in AECOPD reduction and FEV₁ improvement could be accounted for by more pronounced benefits in the subgroup of former smokers (with 45% reduction in AECOPD rate and 0.09 L improvement in FEV₁), which accounted for about 55% of the patient population. In contrast, the remaining 45% of patients who were current smokers derived no benefit in terms of either AECOPD rate or FEV₁.

While subgroup analyses can often be misleading, there is substantial rationale to have confidence in these findings. Most important, the overall analysis had strong trends in clinical endpoints—in terms of both AECOPD and FEV₁ measures across all timepoints—and which could all be accounted for by the more pronounced benefits in a large well-described subgroup, while the remaining patients had no benefit, but did not suffer from a “negative subset” issue. Moreover, the consistency in pronounced benefits attributable to the former smokers across all endpoints is very supportive.

In summary, this is the first study to demonstrate a potential benefit for a biologic therapy in terms of exacerbation rate and lung function when added to standard therapy in former smokers with COPD.

SAR440340 demonstrated a good safety profile in patients with moderate-to-severe COPD. No anti-drug antibody (ADA) patients were identified after treatment. Overall TEAE and SAEs were balanced between SAR440340 and placebo in terms of events and severity. The most frequent adverse events of special interest (AESIs) were infection and injection site reaction. There were slightly more infections in the SAR440340 treatment group. There were no serious AESIs.

TABLE 11
Summary of Efficacy and Safety Results.

| | | | BL EOS ≥ | EL EOS < |
|---|---|---|---|---|
| | | | 250/mm³ | 250/mm³ |
| | Endpoint | ITT | (N = 133) | (N = 210) |
| 1 | AECOPD | 17.60% | 19,60% | 15.40% |
| | (Relative Risk | (−8.3% to | (−25.2% to | (−20.4% to |
| | Reduction Tx | 37.4%) | 48.4%) | 40.6%) |
| | vs pbo) | p = 0.1647 | p = 0.3308 | p = 0.3517 |
| 2.1 | Pre-BD FEV1 | 0.06 | 0.11 | 0.02 |
| | (L) change | (0.01 to | (0.02 to | (−0.03 to |
| | from BL to | 0.10) | 0.21) | 0.07) |
| | week 16-24 | p = 0.0229 | p = 0.0181 | p = 0.4084 |
| | (LS mean diff) | | | |
| 2.2 | Post-BD FEV1 | 0.02 | 0.07 | −0.01 |
| | (L) change from | (−0.03 to | (−0.02 to | (−0.07 to |
| | BL to week 24 | 0.07) | 0.17) | 0.04) |
| | (LS mean diff) | p = 0.4498 | p = 0.1291 | p = 0.5991 |
| 2.3 | Time to 1st | 17.30% | 11.10% | 24.60% |
| | AECOPD | (−11.7% to | (−46.4% to | (−11% to |
| | (Relative | 38.7%) | 45.9%) | 48.7%) |
| | Reduction | p = 0.2155 | p = 0.6449 | p = 0.1525 |
| | Tx vs pbo) | | | |
| 3 | SGRQ total score | −0.516 | −3.13 | 1.03 |
| | chance from 131 | (−3.66 to | (−8.32 to | (−2.73 to |
| | to week 24 (LS | 2.43) | 2.06) | 4.79) |
| | mean diff) (the | p = 0.6901 | p = 0.2349 | p = 0.5890 |
| | lower the better, | | | |
| | > = 4 decrease | | | |
| | is considered | | | |
| | as responder) | | | |

| | | |
|---|---|---|
| | | SAR440340 |
| | Placebo | 300mg q2w |
| n (%) | (N = 171) | (N = 172) |
| TEAE | 128 (74.9) | 127 (73.8) |
| Severe TEAE | 26 (15.2) | 23 (13.4) |
| Treatment-emergent SAE | 26 (15.2) | 24 (14.0) |
| TEAE leading to death | 1(0.6) | 2 (1.2) |
| TEAE leading o permanent | 7(4.1) | 9 (5.2) |
| treatment discontinuation | | |
| Treatment-related TEAE | 10 (5.8) | 16 (9.3) |
| Any treatment-emergent AESi | 14 (8.2) | 18 (10.5) |
| Anaphylatic reaction (medically | 0 | |
| reviewed) | | |
| Hypersensibility (medically reviewed) | 2 (1.2) | 2 (1.2) |
| Injection site reaction (serious or severe | 1 (0.6) | 2 (1.2) |
| and lasting 24 hours or longer) | | |
| Infection | 8 (4.7) | 13 (7.6) |
| Parasitic infection | 0 | 1 (0.6) |
| Opportunistic infection | 1 (0.6) | 2 (1.2) |
| Potential drug-related hepatic disorders | 1 (0.6) | 1 (0.6) |
| Malignancy | 3(1.8) | 2 (1.2) |
| Pregnancy | 0 | 0 |
| Symptomatic overdose with IMP | | |
| Symptomatic overdose with NIMP | 0 | |
| Any treatment-emergent other AE | 5 (2.9) | 9 (5.2) |
| grouping event | | |
| Injection site reaction | 5 (2.9) | 9 (5.2) |

### Treatment-Emergent Adverse Event, TEAL; SAE, Serious Adverse Events.

Table 12 below is a summary of efficacy analyses of SAR440340 in COPD overall and among former smokers. Table 13 below is a summary of efficacy analyses of SAR440340 in COPD overall and among current smokers. Data is presented for relative rate reduction of exacerbations, pre-BD and post-BD FEV1, and St. George's Respiratory Questionnaire (SGRQ).

TABLE 12
Summary of Efficacy Analyses of SAR440340 in COPD Overall and Among Former Smokers.

| | | | Exacerbations, | FEV1, L | | SGRQ, |
|---|---|---|---|---|---|---|
| | | | RRR | (p value) | | Units |
| | | No. | (p value) | Pre-BD | Post-BD | (p value) |
| Overall mITT population | | 343 | 17.6% | 0.06 | 0.02 | −0.62 |
| | | | (0.1647) | (0.0229) | (0.4498) | (0.6901) |
| Former | All former smokers | 187 | 41.9% | 0.09 | 0.06 | 1.41 |
| smokers | | | (0.0086) | (0.0072) | (0.0957) | (0.4894) |
| | + screening Eo ≥250/μL | 105 | 30.0% | 0.13 | 0.10 | −1.3 |
| | | | (0.2092) | (0.0193) | (0.0605) | (0.6448) |
| | + screening Eo >250/μL | 82 | 56.9% | 0.05 | 0.01 | 4.25 |
| | | | (0.0032) | (0.1753) | (0.7641) | (0.1523) |
| | + baseline Eo ≥250/μL | 81 | 52.2% | 0.15 | 0.13 | 2.25 |
| | | | (0.0178) | (0.0255) | (0.0651) | (0.4891) |
| | + baseline Eo <250/μL | 106 | 38.4% | 0.04 | 0.01 | 1.33 |
| | | | (0.0641) | (0.1514) | (0.8373) | (0.6222) |
| | + triple therapy | 123 | 40.3% | 0.03 | 0.00 | 3.58 |
| | | | (0.0323) | (0.3832) | (0.9565) | (0.1369) |
| | + double therapy | 64 | 41.6% | 0.20 | 0.19 | −2.25 |
| | | | (0.1555) | (0.0014) | (0.0019) | (0.5588) |
| | Former smokers excluding asthma | 183 | 37.7% | 0.09 | 0.06 | 1.51 |
| | | | (0.0211) | (0.0075) | (0.0932) | (0.4669) |

Blue = clinically meaningful effect size
Red = nominal p-value < 0.05

RRR, relative rate reduction; BD, bronchodilator; FEV1, forced expiratory volume in 1 second; SGRQ, St. George's Respiratory Questionnaire; Eo, eosinophils.

TABLE 13
Summary of Efficacy Analyses of SAR440340 in COPD Overall and Among Current Smokers.

| | | | Exacerbations, | FEV1, L | | SGRQ, |
|---|---|---|---|---|---|---|
| | | | RRR | (p value) | | Units |
| | | No. | (p value) | Pre-BD | Post-BD | (p value) |
| Overall mITT population | | 343 | 17.6% | 0.06 | 0.02 | −0.62 |
| | | | (0.1647) | (0.0229) | (0.4498) | (0.6901) |
| Current | All Current smokers | 156 | −12.0% | 0.02 | −0.01 | −2.51 |
| smokers | | | (0.5622) | (0.5399) | (0.7005) | (0.2955) |
| | + screening Eo ≥250/μL | 67 | 31.7% | 0.02 | −0.05 | −5.61 |
| | | | (0.3439) | (0.766) | (0.4612) | (0.169) |
| | + screening Eo >250/μL | 89 | −2.0% | 0.03 | 0.02 | −0.24 |
| | | | (0.9374) | (0.4462) | (0.7049) | (0.9369) |
| | + baseline Eo ≥250/μL | 52 | −16.3% | 0.07 | 0.01 | −10.79 |
| | | | (0.6475) | (0.3275) | (0.8968) | (0.022) |
| | + baseline Eo <250/μL | 104 | −8.9% | 0.00 | −0.02 | 1.41 |
| | | | (0.7279) | (0.9567) | (0.5586) | (0.6068) |
| | + triple therapy | 103 | −22.0% | −0.01 | −0.04 | −3.58 |
| | | | (0.3697) | (0.8946) | (0.3486) | (0.2608) |
| | + double therapy | 53 | 13.9% | 0.08 | 0.04 | −1.02 |
| | | | (0.7042) | (0.1504) | (0.5294) | (0.7727) |
| | Current smokers excluding asthma | 150 | −7.5% | 0.03 | −0.01 | −2.26 |
| | | | (0.7194) | (0.4336) | (0.7881) | (0.3542) |

Blue = clinically meaningful effect size
Red = nominal p-value < 0.05

RRR, relative rate reduction; BD, bronchodilator; FEV1, forced expiratory volume in 1 second; SGRQ, St. George's Respiratory Questionnaire; Eo, eosinophils.
Results from Treatment and Post-Treatment Periods

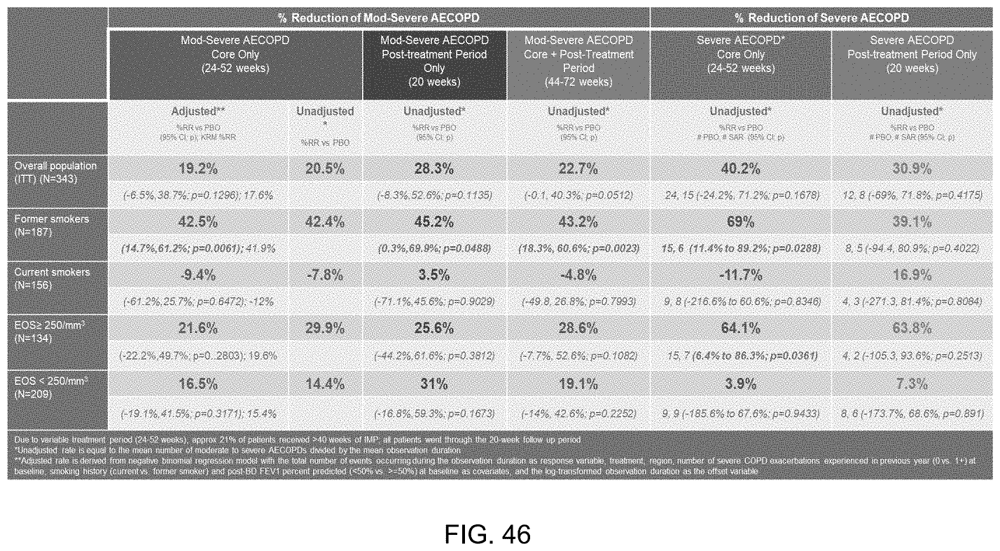

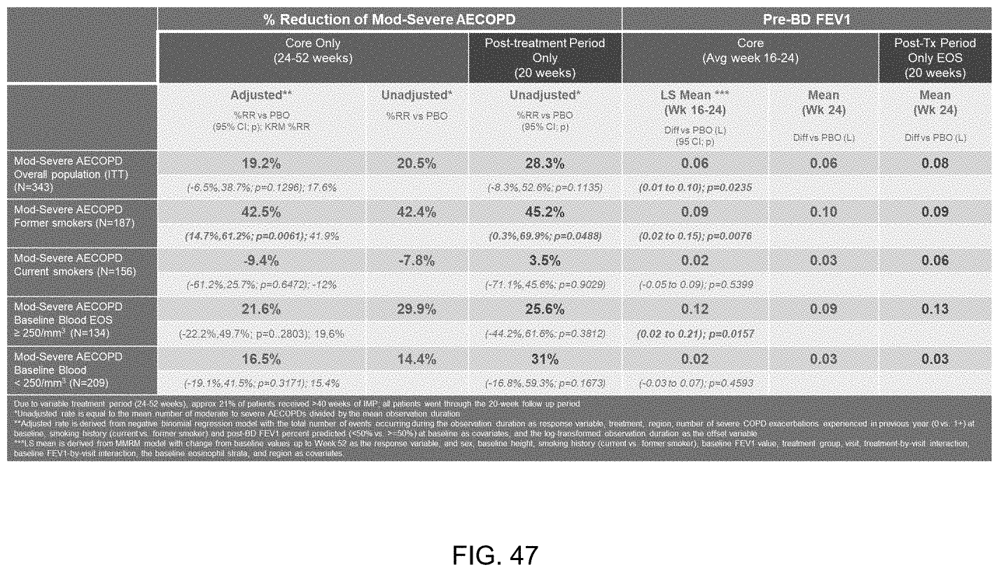

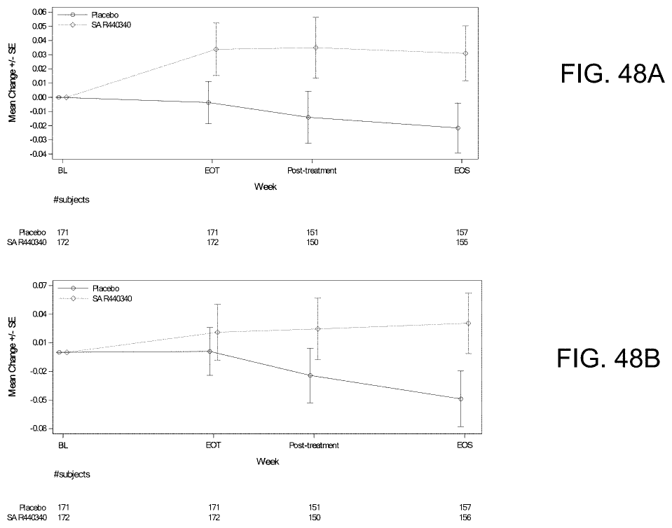

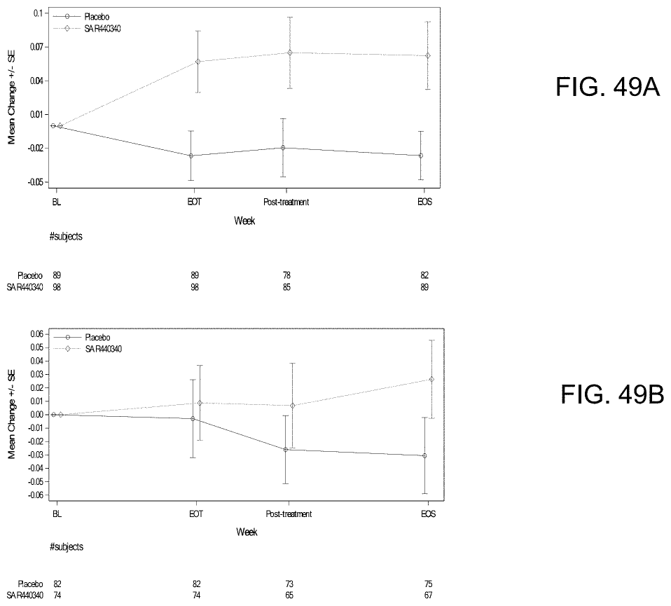

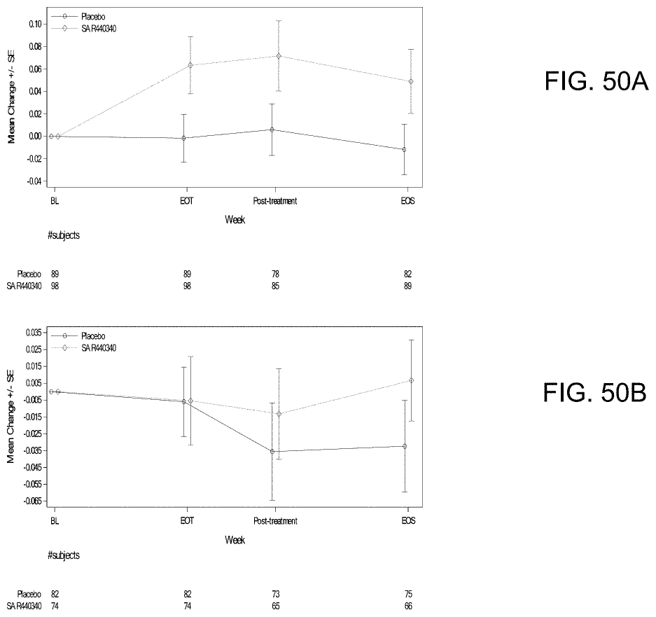

Moderate-to-severe and severe AECOPD during the core and post-treatment periods are shown in FIG. 46. Moderate-to-severe AECOPD and pre-BD FEV1 during the core and post-treatment periods are shown in FIG. 47. The pre-BD FEV1 improvement was sustained during the core and post-treatment periods. Post-BD FEV1 and pre-BD forced vital capacity (FVC) changes for the core and post-treatment periods are shown for the overall intent-to-treat (ITT) population at FIG. 48A-FIG. 48B. A sustained improvement was observed in post-BD FEV1 and pre-BD FVC in the SAR440340 treated patients, while a decline was observed in the placebo arm. Pre-BD FEV1 in the core and post-treatment periods are shown for former and current smokers at FIG. 49A-FIG. 49B. Post-BD FEV1 in the core and post-treatment periods are shown for former and current smokers at FIG. 50A-FIG. 50B. Similar to the overall population, there was sustained effect through the post-treatment period, but a noticeable decline in the placebo group.

### Pharmacokinetics (PK)/Pharmacodynamics (PD) Analysis

Preliminary PK/PD analysis revealed that the treatment response observed with FEV1 appeared to be decoupled from the PK change.

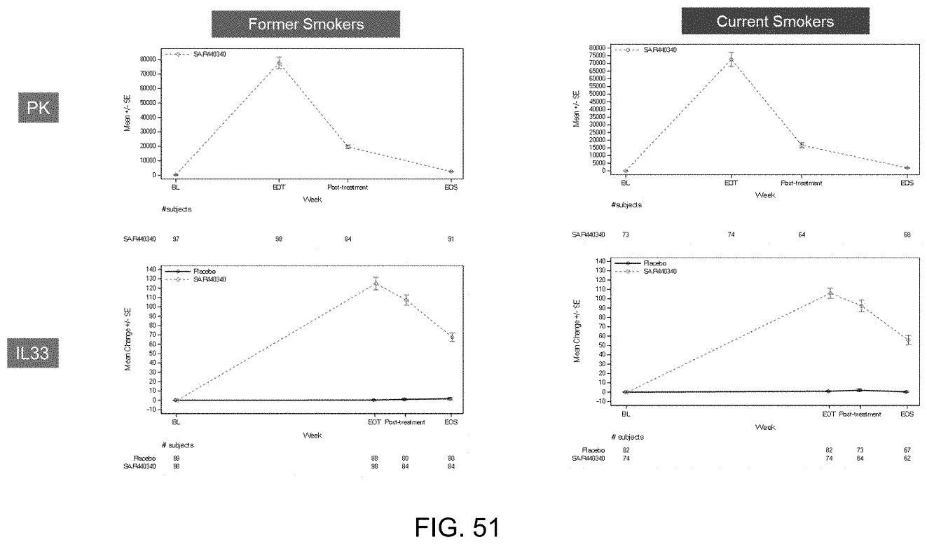

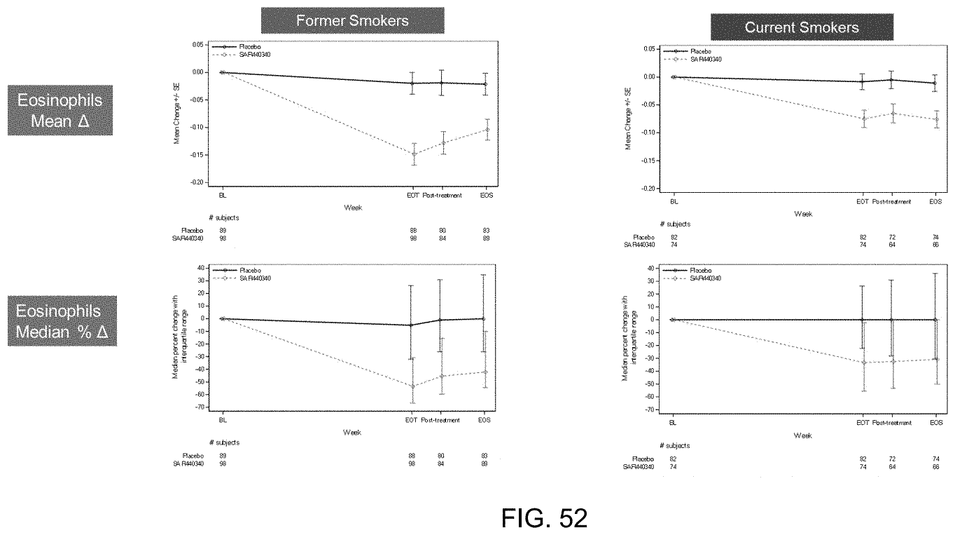

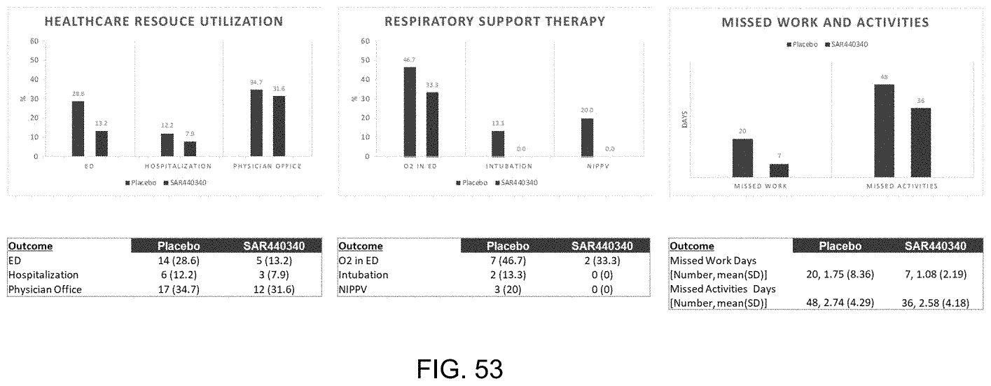

PK/PD during the core and post-treatment periods by smoking subgroup is shown in FIG. 51. Without intending to be bound by scientific theory, the slightly lower IL-33 levels observed in current smokers was unlikely to account for differential efficacy. Blood eosinophil levels during the core and post-treatment periods by smoking subgroup are shown in FIG. 52. Blood eosinophil levels were reduced in both former and current smokers, with the latter having an overall blunted response. AECOPD-related clinical outcomes in former smokers during the core treatment period are shown in FIG. 53. Reduced health care resource utilization (HCRU), respiratory support therapy (e.g., oxygen), and missed work/activities days were observed.

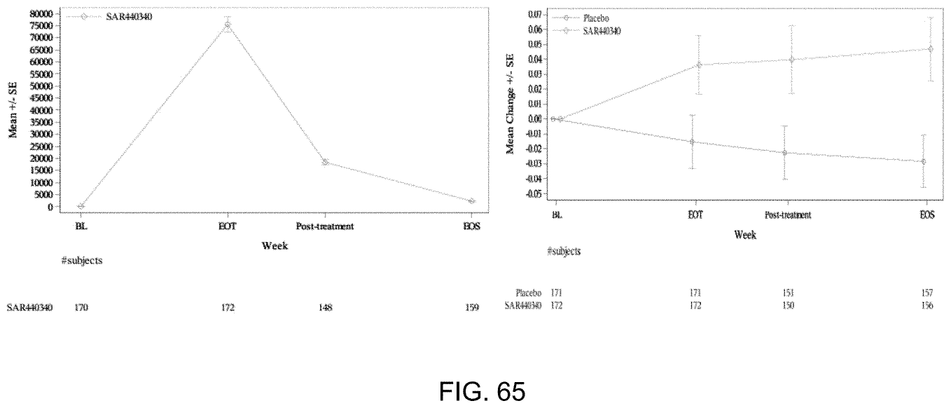

FIG. 65 A comparison of PK and FEV1 in the ITT population was performed (FIG. 65). PK declines, as expected for two compartment PK. The FEV1 mean change from baseline remained flat from end of treatment (EOT) to end of study (EOS). Treatment effect as FEV1 mean change from baseline (active treatment—placebo (SOC)) appeared to slightly further increase during the follow-up, since for placebo, the standard-of-care (SOC) effect declined with time. Overall, FEV1 did not follow a direct effect relationship with PK. The effect offset appeared highly delayed.

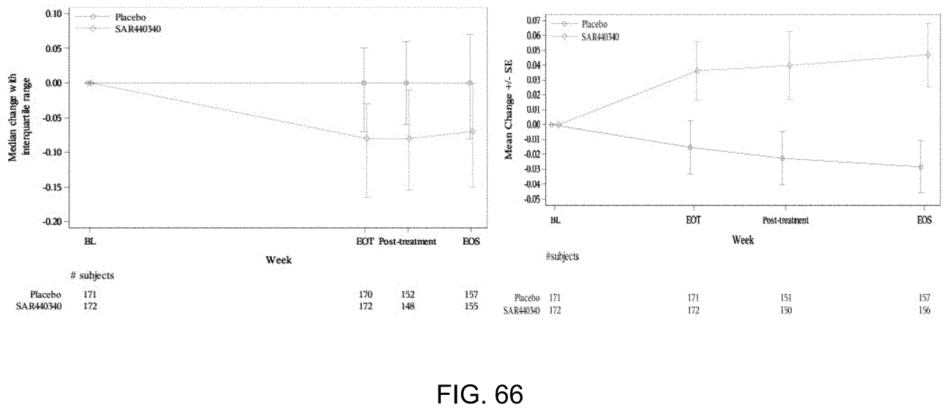

A comparison of EOS and FEV1 in the ITT population was performed (FIG. 66). EOS change from baseline showed a trend of return towards baseline during the follow-up.

PK data from all studies was modelled using a two compartment PK model, which described the data reasonably. Bioavailability was estimated as 53%. Body weight was identified as a major covariate affecting PK. COPD was tested as a covariate only on CL and V2. An effect on V2 (19% lower in COPD) was identified. Other disease-specific factors were not identified as covariates.

Total TL-33 vs. time was modelled using a target mediated drug disposition (TMDD) approach. All studies were included in the analysis. PopPK predicted PK for each individual was used to drive the total IL-33 dynamics. Based on the analysis, a KD=701 nM (95% CI, 6.2-7.9) was estimated, which gives a threshold structure-activity relationship (SAR) concentration of 9.5 mg/L (95% CI, 8.3-10.5) to meet 90% IL-33 engagement. (This was based on the baseline IL-33 assumption of ½ lower limit of quantification (LLOQ).) Based on the computed threshold, 300 mg Q2W, 300 mg Q4W, and 300 mg Q8W may be able to meet the threshold target for achieving 90% target engagement.

### Conclusions

Overall, population PK modelling results show body weight as a significant covariate affecting PK. The PD biomarker profile (IL-33) was delayed vs. the PK profile. FEV1 change and AECOPD did not link to PK directly during the follow-up; there were significantly prolonged delay effects.

### Primary and Secondary Efficacy Endpoints

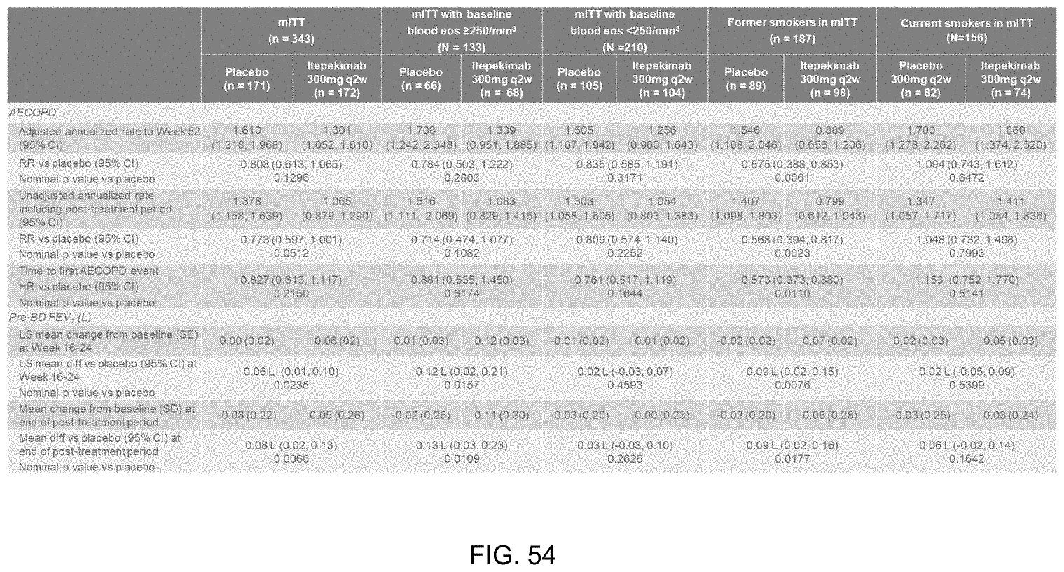

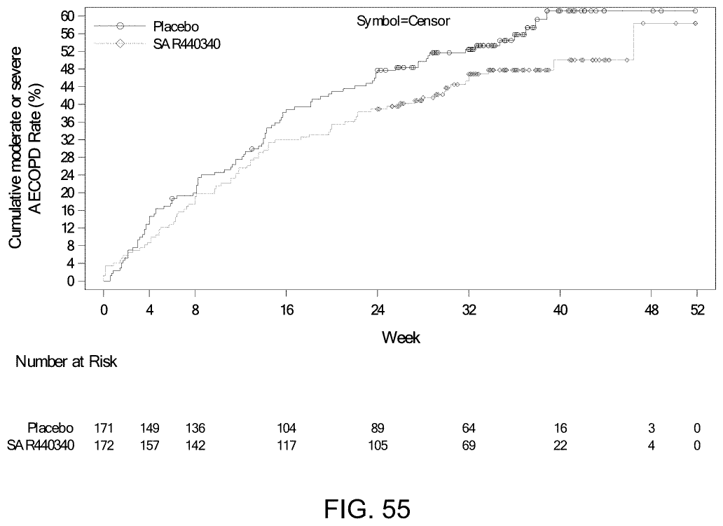

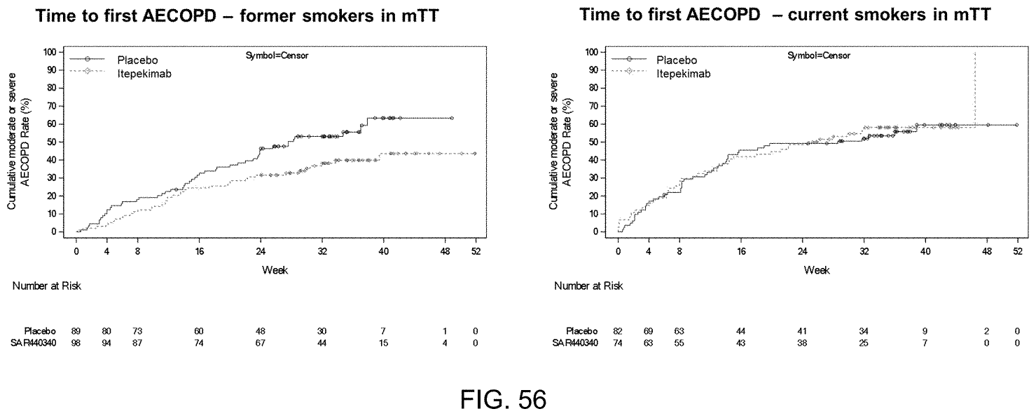

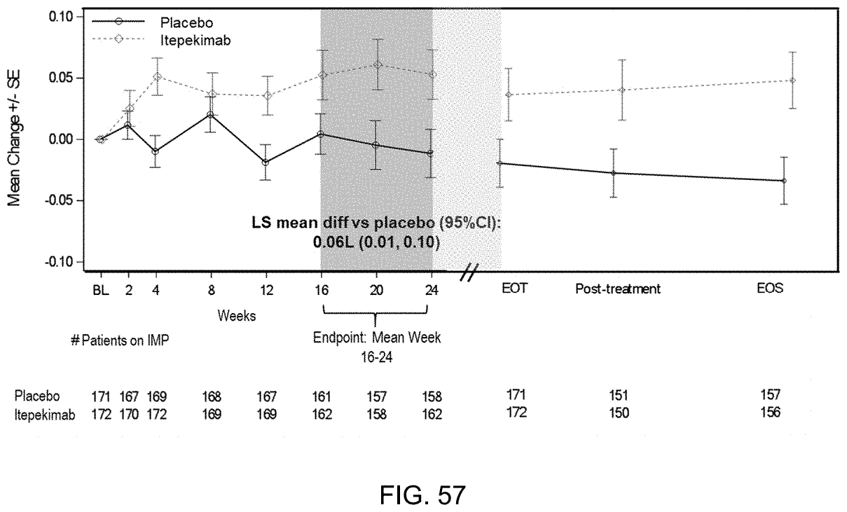

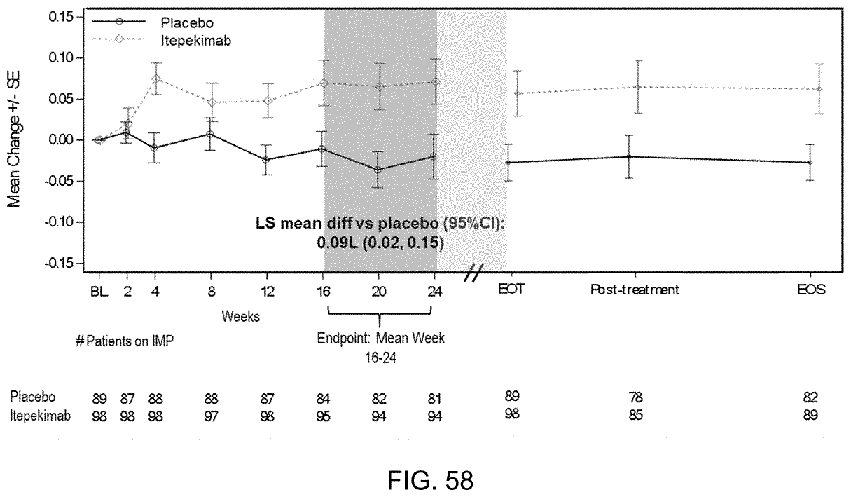

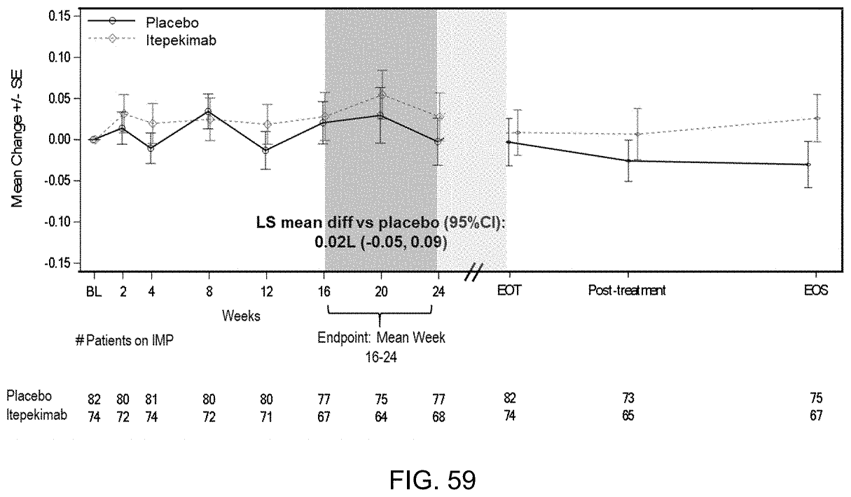

Results for a modified intent-to-treat (mITT) population, a population having a baseline eosinophil level of greater than or equal to 250 mm³, a population having a baseline eosinophil level of less than 250 mm³, former smokers and current smokers are shown at FIG. 54. Time to first AECOPD in an mITT population is shown at FIG. 55. Time to first AECOPD in former smokers (left panel) and current smokers (right panel) is shown at FIG. 56. Change from baseline in pre-BD FEV1 in an mITT population is shown at FIG. 57. Change from baseline in pre-BD FEV1 in former smokers in an mITT population is shown at FIG. 58. Lung function over time in current smokers as a change from baseline in pre-BD FEV1 in current smokers in an mITT population is shown at FIG. 59.

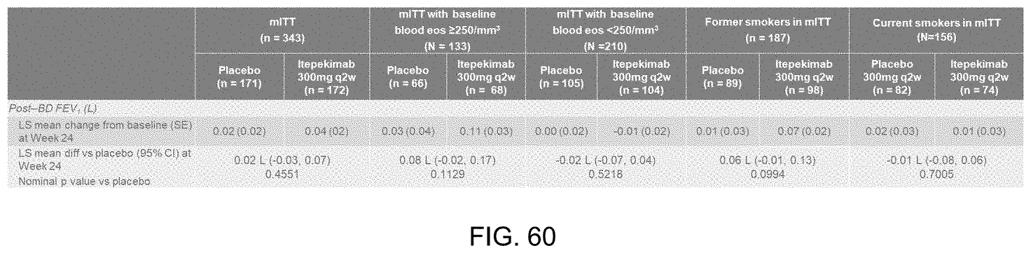

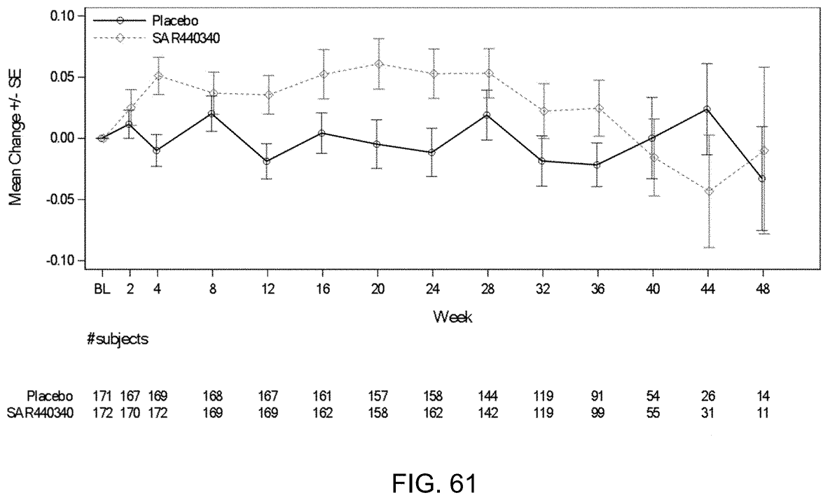

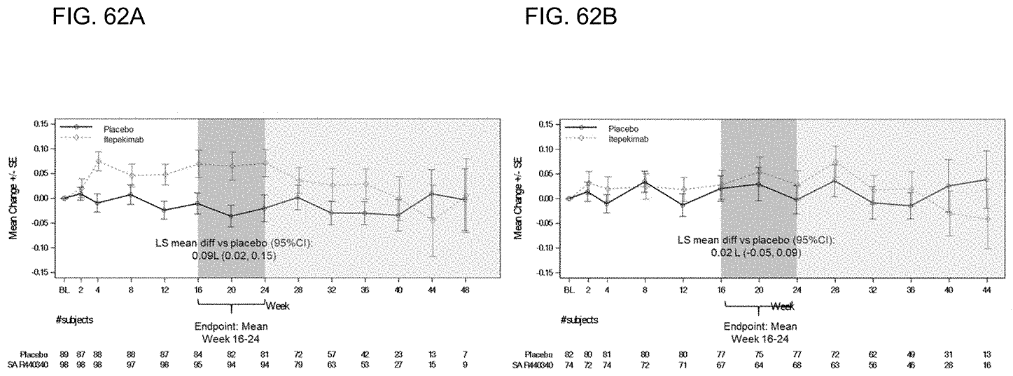

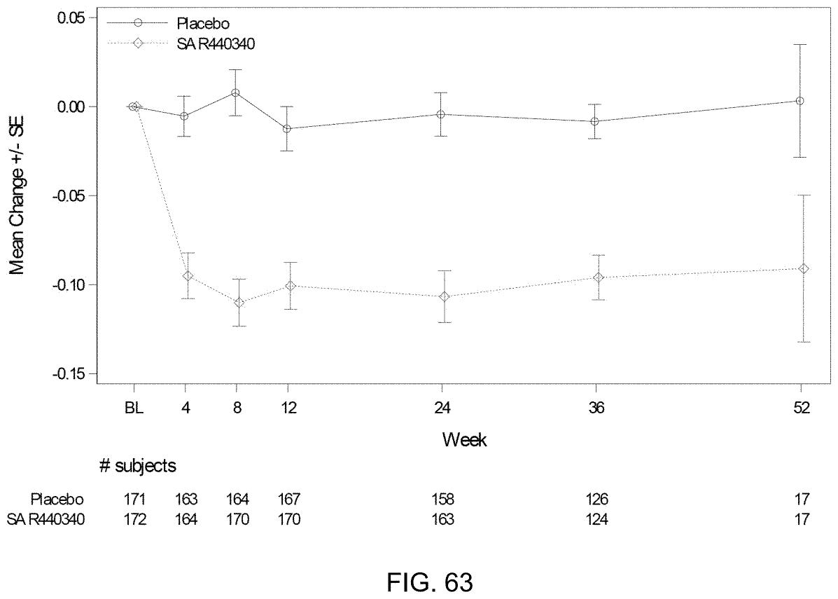

Post-BD FEV1 results at week 24 (mITT, baseline eosinophils <250 or ≥250/mm3, former/current smokers) are shown at FIG. 60. Lung function over time in an mITT population is shown at FIG. 61. Lung function over time in former smokers and current smokers is shown at FIG. 62A-FIG. 62B. The mean change from baseline in blood eosinophil count (10⁹/mL), in a safety population is shown at FIG. 63.

In this novel study with variable treatment duration, including patients with both high and low baseline eosinophils, SAR440340 was associated with a numerically lower annualized rate of moderate-to-severe AECOPD, and a numerically longer time to first moderate or severe AECOPD compared to placebo, and nominal improvement in pre-BD FEV1 from baseline to weeks 16-24 in the mITT population. In patients with high baseline blood eosinophil counts, SAR440340 treatment was associated with nominal improvement in pre-BD FEV1 from baseline to weeks 16-24. In former smokers in the mITT, SAR440340 compared to placebo was associated with nominal improvement in the rate of moderate or severe AECOPD and time to first moderate or severe AECOPD compared to placebo, and nominal improvement in pre-BD FEV1 from baseline to weeks 16-24. These effects were not observed in the population of current smokers in the mITT population.

SAR440340 was generally well tolerated with an acceptable safety profile. Incidences of TEAEs and SAEs were balanced across SAR440340 and placebo treatment groups.

### Example 2. Genetic Association with Serum IL-33 Protein Levels

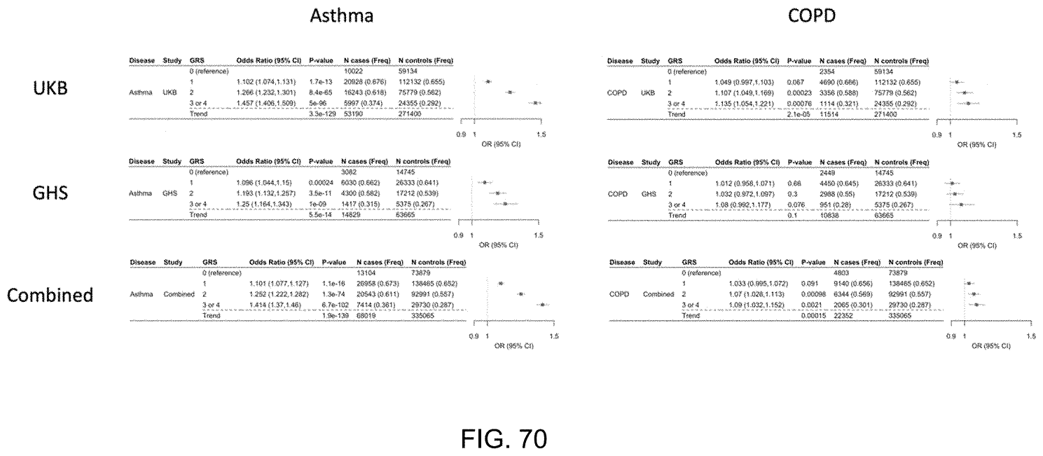

In two independent human genetics studies (involving approximately 100,000 subjects in the Geisinger study including approximately 11,000 with COPD, and approximately 450,000 subjects in the UK Biobank with approximately 11,000 with COPD), a rare IL33 LOF (splice) variant that has been associated with reduced risk of asthma, as well as two common GOF variants in IL33 and its receptor IL1RL1 previously associated with increased asthma risk, were evaluated. After confirming the expected associations with asthma, similar but weaker associations were observed with COPD. The rare LOF variant was associated with a 21% reduction in odds of COPD (meta-analysis p=0.005) and the two common GOF variants with increased odds of COPD, individually (meta-analysis p<0.05 for each variant) and in aggregate (trend p=0.0001) (FIG. 70), demonstrating a gene dosage effect with increasing genetic score of GOF variants conferring increasing COPD risk. These associations supported evaluating the role of IL-33 blockade in COPD.

Total IL-33 concentrations were measured in serum from 437 individuals (53% female) from the Geisinger Health System (GHS) that had been previously genotyped. To increase power to detect an association with rs146597587, the sample was enriched for heterozygous carriers (115 in total) relative to the population frequency. IL-33 levels were measured using an electrochemiluminescence immunoassay from Meso Scale Discovery (MD, USA). The method involved acid treatment of samples to dissociate IL-33 complexed with endogenous binding partners, enabling detection of total IL-33 levels in serum. The assay utilized a biotinylated anti-human IL-33 monoclonal antibody as the capture reagent and a recombinant human IL-33 as the standard. Captured IL-33 was detected using a ruthenium-labeled anti-human IL-33 monoclonal antibody. The assay was specific for the reduced form of IL-33 and had a sensitivity of 6.25 pg/mL in neat human serum. Differences in IL-33 levels between homozygous and heterozygous carriers were tested using linear regression, with age, sex and asthma case-controls status included as covariates.

Genetic Association with Eosinophil Counts, Asthma and COPD

Genetic analyses of common gain-of-function (GOF) variants and a rare loss-of-function (LOF) variant in the IL-33 pathway, previously associated with asthma risk, were characterized for COPD risk. Association analyses were performed in individuals of genetically-confirmed European ancestry from two studies described previously, the UK Biobank (UKB) and GHS studies.

UKB Study

Eosinophil counts (N=448,848) were normalized using a rank-based inverse-normal transformation and tested for association with imputed variants released by the UKB using BOLT-LMM v0.4. Age, age2, sex, age-by-sex, age2-by-sex and 10 ancestry-informative principal components were included as covariates. Asthma cases (N=53,190) were individuals (i) with a self-reported doctor diagnosis (data-fields 6152 and 20002) or ICD10 code for asthma (J45 or J46 in data-fields 41270 or the GP clinical table); and (ii) no COPD (see below), emphysema or chronic bronchitis (based on data-fields 20002, 22128 to 22130). COPD cases (N=11,514) were individuals with: (i) with a self-reported doctor diagnosis (data-fields 6152 and 20002) or ICD10 code for COPD (J41, J42, J43 or J44); and (ii) no asthma (see above). A common set of controls (N=271,400) was used for both asthma and COPD; these were individuals (i) without asthma, COPD, other respiratory or allergic conditions, based on ICD10 codes and data-fields 6152, 20002, 22126 to 22130; and (ii) if spirometry data were available, with FEV1/FVC>=0.7 and percent predicted FEV1 >=0.8. Association analyses were performed with SAIGE v0.6 [Zhou 2018], with the same covariates listed above.

GHS Study

The association between eosinophil counts (N=100,413) and imputed variants (reference panel: Haplotype Reference Consortium) was tested with BOLT-LMM as described for the UKB study. For individuals with longitudinal data, we analyzed the median of available observations. Association analysis was performed separately for samples genotyped with two different Illumina arrays (OMNI and GSA), and results combined using an inverse-variance meta-analysis. Asthma cases (N=14,829) were defined as individuals with an ICD10 code for asthma but not COPD, and the reverse for COPD cases (N=10,838). Controls (N=63,665) for both analyses were individuals (i) without asthma, COPD, other respiratory or allergic conditions, based on ICD10 codes; (ii) not taking medication for respiratory diseases; and (iii) with no spirometry data available, because spirometry testing (irrespective of the outcome) was found to be a predictor of respiratory disease. Association analyses were performed with SAIGE.

Meta-Analysis of UKB and GHS Studies

Association results were combined with an inverse-variance meta-analysis using METAL. The genomic inflation factor (i.e. lambda) for common variants (frequency >1%) was 1.57 for eosinophil counts, 1.18 for asthma and 1.07 for COPD. The corresponding intercepts from LD-score regression were 1.21, 1.15 and 1.02.

Mendelian Randomization Analysis

The causal effect of protein levels of interleukin 1 receptor-like 1 (IL-33R, ST2) on disease risk was estimated using the inverse-variance weighted method described by Burgess et al. (Stat Med. 2016; 35(11):1880-1906). The instrumental variables were rs10179654 (2:102305323:T:G, minor allele frequency [MAF]=48%, located 6 Kb upstream of IL1RL1) and rs13029918 (2:102340831:A:G, MAF=3%, located in a splice region), which reduce plasma levels of IL-33R respectively by 0.85 (for the G allele; P=10-391) and 1.28 (for the G allele; P=10-213) SD units (Sun (2018) Nature. 558(7708):73-79).

Human Genetic Studies

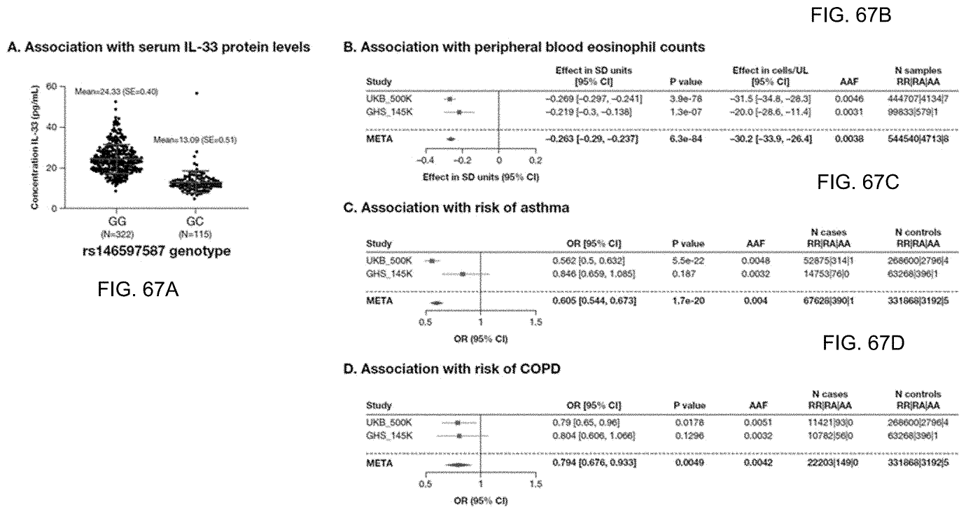

To study the association between IL-33 and COPD, a rare splice-acceptor allele (rs146597587:C, 0.4% frequency in Europeans) that results in a truncated IL-33 isoform that does not bind the IL-33 receptor and lowers total IL33 mRNA by 40% and reduces asthma risk by about 50% (Smith PLoS Genet. 2017; 13(3):e1006659) was investigated. Heterozygous individuals had a 46% reduction in serum IL-33 protein levels compared to non-carriers (FIG. 67A) and confirmed the reported (Id.) reduction in peripheral blood eosinophil counts (−0.26 standard deviation [SD] units in the UK Biobank study, −30 cells/uL) (FIG. 67B) and protection from asthma (39% risk reduction) (FIG. 67C). In a meta-analysis including 22,352 COPD cases and 335,065 controls a 21% reduction in disease risk was discovered (odds ratio [OR]=0.794, 95% CI=0.676-0.933, P=0.0049) (FIG. 67D).

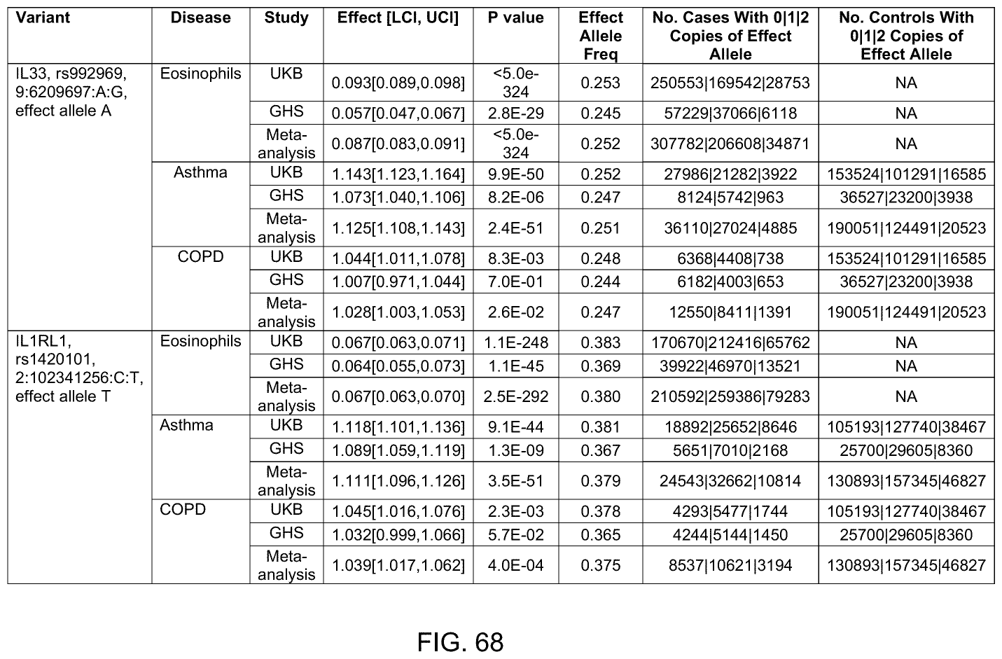

Next a common intronic variant in IL-33 (rs992969:G, 75% frequency) that lowers total IL-33 mRNA in bronchial epithelial cells by 4% (Ketelaar J Allergy Clin Immunol. 2020; 50091-6749(20)30680-1), eosinophil counts by 0.09 SD units (9 cells/uL), and asthma risk by 13%, was investigated (FIG. 68). This allele was associated with a 3% reduction in COPD risk (OR=0.973, 95% CI=0.950-0.997, P=0.026).

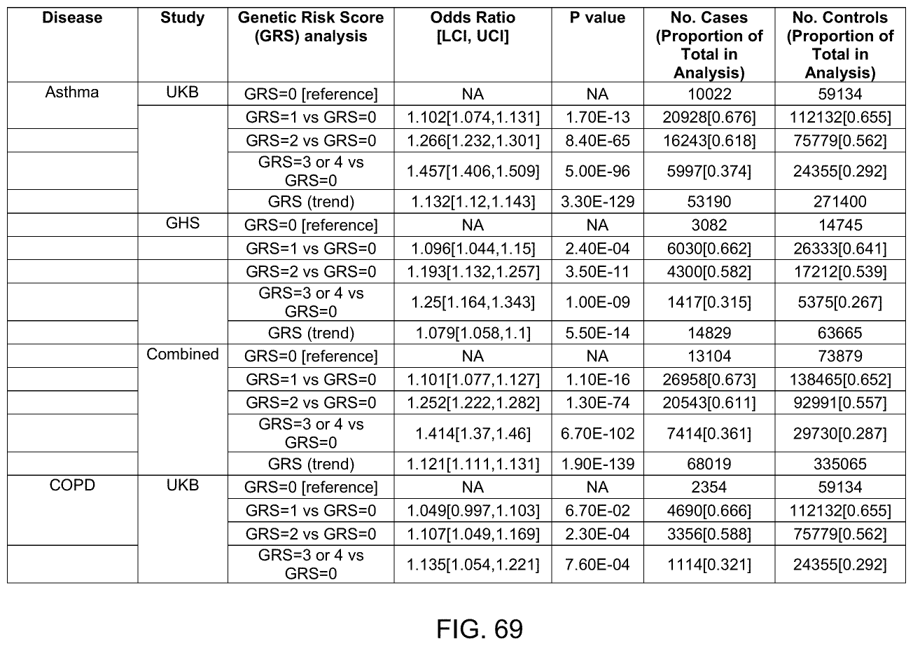

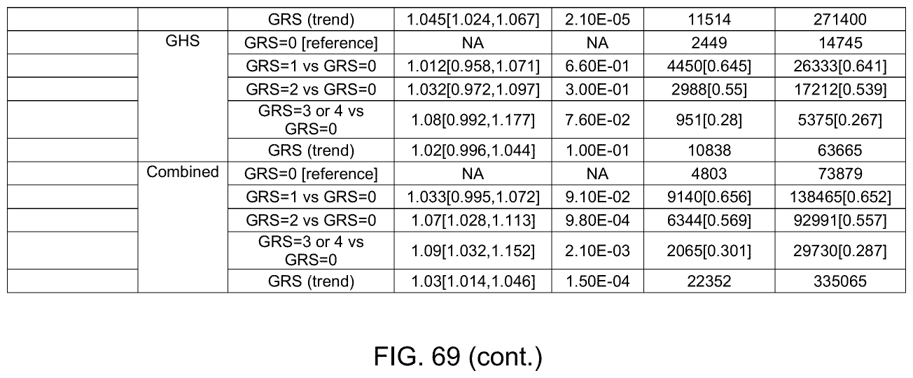

Lastly, two common variants that increase plasma levels of soluble IL-33R (ST2, a decoy receptor for IL-33) by 0.85 SD units (rs10179654:T) and 1.28 SD units (rs13029918:A), respectively (Sun, Supra), were investigated. Based on Mendelian randomization analysis using these two variants as instrumental variables, it was discovered that a one SD unit increase in soluble IL-33R levels was associated with a 3% reduction in COPD risk (OR=0.969, 95% CI=0.948-0.991, P=0.0061) (FIG. 69).

Genetic analyses demonstrated association of LOF in IL33 with reduced COPD risk, and GOF in IL33 pathway (IL33 and the IL-33 receptor IL1RL1) variants with increased risk. In a randomized trial with placebo and itepekimab, AECOPD was 1.61 and 1.30 (relative risk [RR]0.81; 95% CI 0.61-1.07), and least squares mean (LSM) pre-bronchodilator forced expiratory volume in one second (FEV1) change to weeks 16-24 was 0.00 L and 0.06 L (LSM difference 0.06 L; 95% CI 0.01-0.10). Both AECOPD reduction and FEV1 improvement in the overall population were accounted for by more pronounced benefit with itepekimab in former smokers, nominally significant reduction in AECOPD (0.58; 0.39-0.85) and FEV1 improvement (0.09 L; 0.02-0.15). Current smokers showed no significant benefit in exacerbations (1.09; 0.74-1.61) or FEV1.

Together, these genetic findings are consistent with IL-33 blockade and protection from COPD.

### Example 3. A Randomized, Double-Blind, Placebo-Controlled, Parallel-Group, Phase 3 Study to Evaluate the Efficacy, Safety and Tolerability of SAR440340/REGN3500/Itepekimab (Anti-IL-33 mAb) in Former Smokers with Moderate-to-Severe Chronic Obstructive Pulmonary Disease (COPD) (AERIFY-1)

### Overall Design

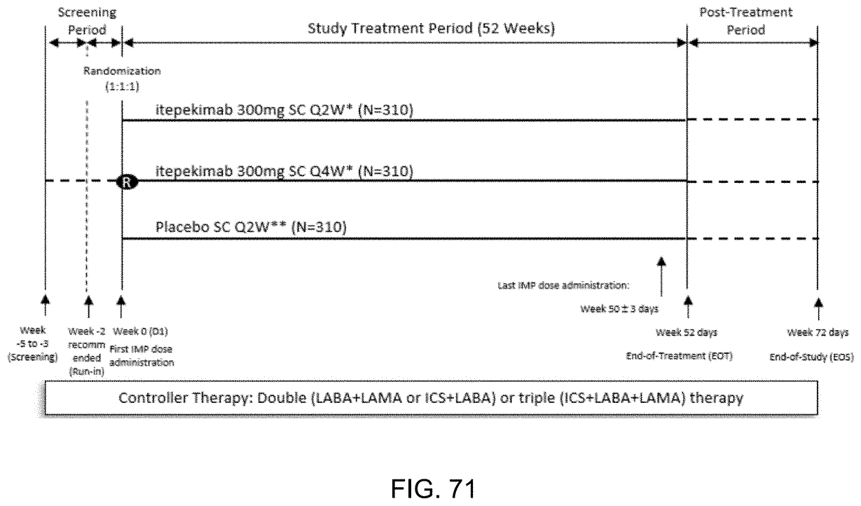

This is a multinational, randomized, double-blind, placebo-controlled, parallel-group (3 groups), 52-week, Phase 3 study to assess the efficacy, safety, and tolerability of two dosing regimens of itepekimab in patients with moderate-to-severe COPD who are former smokers and are on an established double (ICS+LABA or LAMA+LABA) or triple controller therapy (LAMA+LABA+ICS). Study treatments are itepekimab 300 mg every 2 weeks (Q2W), itepekimab 300 mg every 4 weeks (Q4W), or matching placebo administered subcutaneously (SC) during the 52-week treatment period. The study design is graphically depicted at FIG. 71.

The primary efficacy endpoint is the annualized rate of moderate or severe acute exacerbations of COPD (AECOPD) over the 52-week placebo-controlled treatment period. Moderate exacerbations are recorded by the investigator and are defined as acute worsening of respiratory symptoms that require either systemic corticosteroids (such as intramuscular (IM), intravenous (IV), or oral) and/or antibiotics. Severe exacerbations are recorded by the investigator and are defined as AECOPD that require hospitalization, observation for greater than 24 hours in an emergency department/urgent care facility, or result in death. For both moderate and severe events to be counted as two separate events, they are separated by at least 14 days between any course of systemic steroids/antibiotics or 14 days between discharge and new admission in case of hospitalization (severe events only).

For efficacy endpoint analysis, the primary population is the intent-to-treat (ITT) population. In addition to the analysis in the current study, statistical analysis for subpopulation of participants with triple controller therapy will be further conducted using the pooled data of this Example and the data obtained in Example 4.

Randomization is stratified by country (some countries might be pooled together), screening blood eosinophil counts (<300 cells/mm³ or ≥300 cells/mm³), and controller therapy (double or triple) at baseline. To ensure enrollment according to intended distribution of controller therapy and eosinophil count, the number of participants enrolled into each stratification group is controlled and monitored as follows:
Double controller therapy (ICS+LABA or LAMA+LABA): up to approximately 35% of participants.
Eosinophils ≥300 cells/mm³ approximately 35% of participants.

The study duration is outlined below:
Screening period (3-5 weeks).
Randomized investigational medicinal product (IMP) treatment period (52 weeks).
Post-IMP treatment follow-up period (20 weeks).

Participants have been on a SoC controller therapy for COPD for at least 3 months prior to screening (visit 1A) with a stable dose for controller therapy for ≥1 month prior to screening (visit 1A) and the screening period and stay on their established controller medication for COPD throughout the duration of the study, with the exception of systemic steroids and antibiotics used for AECOPD.

Participants who satisfy the inclusion criteria are randomized (1:1:1) to one of the following IMP treatment groups to be administered for 52 weeks:
Itepekimab 300 mg, administered as a single subcutaneous (SC) injection Q2W.
Itepekimab 300 mg, administered as a single SC injection Q4W with alternating SC injection of matching placebo at the 2-week interval between active IMP.
Placebo, administered as a single SC injection of matching placebo to itepekimab Q2W.

### Number of Participants:

Approximately 930 participants are randomized 1:1:1 into the 3 treatment arms. Approximately 310 participants are randomized per arm to receive either itepekimab 300 mg Q2W, itepekimab 300 mg Q4W, or matching placebo to itepekimab.

### Intervention Groups and Duration:

There are 3 arms:
Arm A: Itepekimab 300 mg SC Q2W.
Arm B: Itepekimab 300 mg SC Q4W.
Arm C: Matching placebo SC Q2W.

Participants are treated for 52 weeks.

### Type of Participant and Disease Characteristics:

Participants have a physician diagnosis of COPD for at least 1 year (based on the GOLD definition).

Participants have a smoking history of ≥10 pack-years, but are not currently smoking, and smoking cessation occurred ≥6 months prior to screening (visit 1A) with an intention to quit permanently. Urine cotinine levels are tested at screening (visit 1A) and each subsequent visit during the study.

Participants have moderate-to-severe COPD with post-BD FEV1/FVC ratio ≤0.70 and a post-BD FEV1% predicted ≥30% and <80% at screening (visit 1A) and at baseline/randomization (visit 2).

Participants have a COPD Assessment Test (CAT) score ≥10 at screening (visit 1A) and baseline/randomization (visit 2).

Participants have participant-reported history of signs and symptoms of chronic bronchitis (chronic productive cough for at least 3 months in the year prior to screening in a participant in whom other causes of chronic cough (e.g., inadequately treated gastroesophageal reflux or chronic rhinosinusitis; or clinical diagnosis of bronchiectasis) is excluded).

Participants have a documented history of high exacerbation risk defined as having had ≥2 moderate or ≥1 severe exacerbations within the year prior to screening (visit 1A), with at least 1 exacerbation treated with systemic corticosteroids. At least one exacerbation occurred while participants were on their current controller therapy: moderate exacerbations are recorded by the investigator and are defined as acute worsening of respiratory symptoms that requires either systemic corticosteroids (IM, V, or oral) and/or antibiotics (however, use of antibiotics alone does not qualify as a moderate exacerbation unless documentation is available that use of antibiotics was necessary for treatment of worsening symptoms of COPD); severe exacerbations are recorded by the investigator and are defined as AECOPD that require hospitalization or observation for >24 hours in emergency department/urgent care facility.

Participants use SoC controller therapy, for ≥3 months prior to screening (visit 1A) and at a stable dose of controller therapy for at least 1 month prior to the screening and during the screening period, including either: double therapy (i.e., ICS+LABA or LAMA+LABA), or triple therapy (LAMA+LABA+ICS).

### Study Intervention(s):

### Investigational Medicinal Product(s)

Sterile itepekimab or matching placebo is provided in prefilled syringes for SC administration. Each prefilled syringe contains a deliverable volume of 2 mL with an itepekimab concentration of 150 mg/mL or 0 mg/mL.
Formulation: 2 mL solutions for injection (150 mg/mL).
Route(s) of administration: subcutaneous (SC).
Dose regimen: All participants receive Q2W dosing to maintain the blind. Participants on Q4W will receive alternating doses of IMP and placebo.

### Non-Investigational Medicinal Products(s)

Participants continue on their established controller therapy.
Formulation: Dry powder inhaler (DPI), metered dose inhaler (MDI), or nebulizer.
Route(s) of administration: oral inhalation for LAMA, LABA, ICS, ICS+LABA, LAMA+LABA, or LAMA+LABA+ICS.
Dose regimen: As per prescribed.

### Reliever Medication (Albuterol/Salbutamol, Levalbuterol/Levosalbutamol, Ipratropium, Ipratropium/Albuterol)

Participants may administer albuterol/salbutamol, levalbuterol/levosalbutamol, ipratropium, or ipratropium/albuterol as reliever medication as needed during the study.
Formulation: DPI, MDI, nebulizer.
Route(s) of administration: oral inhalation, nebulization.
Dose regimen: As needed, per prescribed.

### Statistical Considerations:

### Primary Endpoint:

The primary analysis of the annualized rate of moderate or severe AECOPD during the 52-week placebo-controlled treatment period is performed following the ITT principle. The primary estimand is a treatment policy estimand. All moderate or severe AECOPD events during the 52-week treatment period are included and the observation duration are from randomization to visit 28 (week 52). Participants who permanently discontinue IMP are asked and encouraged to return to the clinic for all remaining study visits, all off-treatment moderate or severe AECOPD during the planned 52-week treatment period will be included in the primary analysis. Similarly, if a participant withdraws from the study prior to the end of the 52-week treatment period, all observed moderate or severe AECOPD events up to the last contact date are included in the analysis, and in this case the observation duration is from randomization to the last contact date. No imputation is performed for the unobserved events that may happen after study discontinuation and up to week 52. The annualized rate of moderate or severe AECOPD will be analyzed using a negative binomial regression model. The model will include the total number of moderate or severe AECOPD events that occur during the treatment period (up to week 52) as the response variable, with treatment group (placebo, itepekimab 300 mg SC Q2W, itepekimab 300 mg SC Q4W), region (pooled country), screening eosinophil strata (<300 cells/mm³, ≥300 cells/mm³), controller therapy (double, triple) strata, baseline disease severity (as % predicted post-bronchodilator (BD) FEV1 used as continuous variable), and total number of severe AECOPD events within one year prior to the study (0 or ≥1) as covariates.

Log-transformed observation duration will be the offset variable. The treatment comparisons with placebo is performed using a step-down procedure to compare itepekimab 300 mg SC Q2W versus placebo first. The comparison of itepekimab 300 mg SC Q4W versus placebo be performed only if the comparison is statistically significant.

This estimand compares the rate of moderate or severe AECOPD for the participants randomly assigned to an itepekimab regimen versus placebo, regardless of what treatment the participants actually receive or whether the treatment regimens have been adhered to. It assesses the benefits of the treatment policy or strategy relative to placebo. The estimated annualized event rate for each treatment group and its 2-sided 95% confidence intervals (CIs) will be derived from the negative binomial model. The event rate ratio (RR) of each itepekimab regimen versus placebo, and the corresponding 2-sided 95% CI and p-value will also be provided.

An on-treatment analysis to assess the efficacy of itepekimab excluding data measured when participants do not adhere to the treatment regimen as per protocol is also performed and is used to estimate the benefit when adhering to itepekimab treatment. In this analysis, only the AECOPD events observed during the on-treatment period (from first administration of IMP to last administration of IMP+14 days) are included. Off-treatment events of participants who prematurely discontinue treatment are excluded from the analysis. A negative binomial model with the same set of covariates as specified in the primary analysis is used. This model includes moderate or severe AECOPD occurring during the on-treatment period as the response variable, and the log transformed duration of the treatment period is the offset variable. This approach defines the estimand to evaluate the efficacy of itepekimab while on treatment.

### Secondary Endpoints:

Change from Baseline in Pre-BD FEV1 at Week 52

The main analysis of change from baseline in pre-BD FEV1 at week 52 is to assess the efficacy of itepekimab on pulmonary function. The change from baseline in pre-BD FEV1 at week 52 is analyzed using a mixed-effect model with repeated measures (MMRM) approach. The model includes change from baseline in pre-BD FEV1 values up through week 52 as response variables, and treatment, age (continuous variable (years)), sex, baseline height (continuous variable), region (pooled country), screening eosinophil strata, controller therapy strata (double or triple), visit, treatment by-visit interaction, and baseline pre-BD FEV1 value (continuous variable) and baseline pre-BD FEV1-by-visit interaction as covariates. Participants who discontinue IMP before week 52 are asked and encouraged to return to the clinic for all remaining study visits and the additional off treatment pre-BD FEV1 values measured up through week 52 will be included in the analysis. For participants who withdraw from the study before week 52, pre-BD FEV1 values will be missing after study discontinuation or last contact. No imputation is performed for missing values in this analysis. This estimand compares the change from baseline in pre-BD FEV1 for the participants randomly assigned to an itepekimab regimen versus the participants randomly assigned to the placebo arm, regardless of the treatment participants actually received. It assesses the benefits of the treatment policy or strategy relative to placebo.

An unstructured correlation matrix is used to model the within-participant errors. Parameters are estimated using restricted maximum likelihood method with the Newton-Raphson algorithm. Statistical inferences on treatment comparisons for the change from baseline in pre-BD FEV1 at week 52 is derived from the mixed-effect model. Difference in least squares (LS) mean change from baseline, the corresponding 95% CI and p-value is provided for comparison of each itepekimab regimen against placebo.

To assess the treatment effect when the participants adhere to the study treatment as directed, on-treatment pre-BD FEV1 measurements are analyzed using a similar MMRM model as for the primary pre-BD FEV1 analysis, including the same set of covariates and estimation algorithm. The model includes on-treatment change from baseline in pre-BD FEV1 values up through week 52 as response variables. A pre-BD FEV1 value is considered as on-treatment if it is measured on or before the last dose date+14 days.

### AECOPD

The time to first moderate or severe AECOPD is determined over the 52-week placebo-controlled treatment period. The annualized rate of severe AECOPD is determined over the 52-week placebo-controlled treatment period. The time to first severe AECOPD is determined over the 52-week placebo-controlled treatment period. The annualized rate of corticosteroid-treated AECOPD is determined over the 52-week placebo-controlled treatment period.

### Respiratory Symptoms

The change from baseline in E-RS:COPD (Evaluating Respiratory Symptoms in COPD) total score is determined at week 52.

### FEV1 Slope

The rate of change in post-BD FEV1 (L) from baseline (post-BD FEV1 slope) is determined after 4-12 weeks.

### HRQoL as Assessed by SGRQ

The change from baseline in St. George's Respiratory Questionnaire (SGRQ) total score is determined at week 52. The proportion of participants with a decrease from baseline of at least 4 points in SGRQ total score is determined at week 52.

### Safety and Tolerability

The incidence of treatment-emergent adverse events (TEAEs), adverse events of special interest (AESIs), serious adverse events (SAEs), and adverse events (AEs) leading to permanent treatment discontinuation is determined. The incidence of potentially clinically significant laboratory tests, vital signs, and ECG abnormalities is determined in the treatment-emergent period.

### Pharmacokinetic (PK) Profile

Functional itepekimab concentrations in serum are determined from baseline to the end of the study.

### Immunogenicity

The incidence of treatment-emergent anti-itepekimab antibody responses are determined throughout the study.

### Tertiary/Exploratory Endpoints:

### Healthcare Utilization

Annualized number of days of healthcare resource utilization is determined over the 52-week placebo-controlled treatment period.

### Predictors of Mortality

Annualized number of ER and hospital days related to AECOPD are determined. Proportion of participants with Body Mass Index, Airflow Obstruction, Dyspnea, and Exercise Capacity (BODE) Index score >1 point decrease (=improvement) is determined at week 52.

### Pulmonary Function

The proportion of participants with pre-BD FEV1 improvement ≥100 mL is determined at week 52. The proportion of participants with pre-BD FEV1 improvement ≥100 mL is determined at week 24.

### Reduction in Oral Corticosteroid, Antibiotic Use

The number of days on oral corticosteroids and antibiotics over 52 weeks is determined.

### Respiratory Vital Signs

The change from baseline in resting oxygen saturation is determined at week 52.

### Biomarkers

The change from baseline in blood eosinophil levels and neutrophil levels are determined at weeks 4, 8, 12, 24, 36 and 52. The change from baseline is determined at weeks 4, 12, 24 and 52 for total blood IL-33, and blood C-reactive protein (CRP).

### Gene Expression and Genetic Factors

Pharmacogenomics analyses, DNA sampling, and RNA sampling may be performed.

### Example 4. A Randomized, Double-Blind, Placebo-Controlled, Parallel-Group, Phase 3 Study to Evaluate the Efficacy, Safety and Tolerability of SAR440340/REGN3500/Itepekimab (Anti-IL-33 mAb) in Former Smokers with Moderate-to-Severe Chronic Obstructive Pulmonary Disease (COPD) (AERIFY-2)

### Overall Design

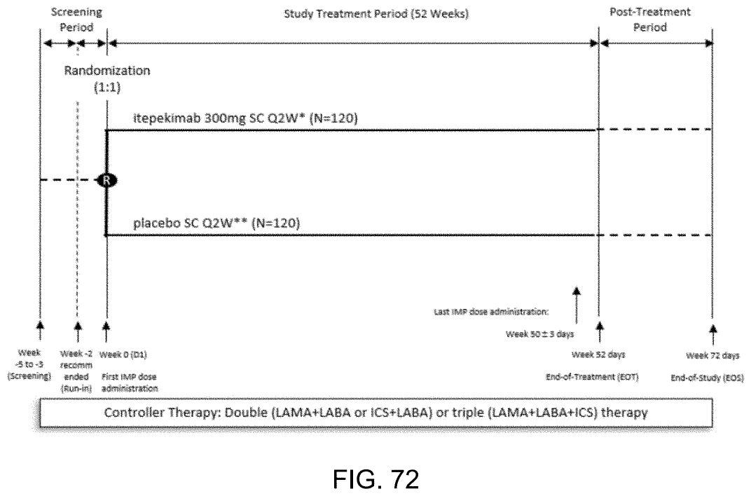

This is a multinational, randomized, double-blind, placebo-controlled, parallel-group, 52-week, Phase 3 study to assess the efficacy, safety, and tolerability of itepekimab in two cohorts. One cohort consists of participants with moderate-to-severe COPD who are former smokers (primary population) (FIG. 71), and one cohort consists of participants with moderate-to-severe COPD who are current smokers (secondary population) (FIG. 72). All the participants from both cohorts receive established triple (LAMA+LABA+ICS) or double controller therapy (LAMA+LABA or ICS+LABA). The aim of the study in the former smoker cohort is to evaluate the efficacy and assess the safety and tolerability of two dosing regimens of itepekimab. Study treatments for former smokers are itepekimab 300 mg every 2 weeks (Q2W), itepekimab 300 mg every 4 weeks (Q4W), or matching placebo administered subcutaneously during the 52-week randomized treatment period (three treatment groups). In addition, the study also estimates the efficacy, safety, and tolerability of a 300 mg Q2W dosing regimen of itepekimab compared to a matching placebo in the cohort of current smokers. Study treatments for current smokers are itepekimab 300 mg Q2W, or matching placebo administered subcutaneously during the 52-week randomized treatment period (2 treatment groups).

The primary efficacy endpoint is the annualized rate of moderate-or-severe acute exacerbations of COPD (AECOPD) over the 52-week placebo-controlled treatment period in former smokers. Moderate exacerbations will be recorded by the investigator and are defined as acute worsening of respiratory symptoms that require either systemic corticosteroids (such as intramuscular (IM), intravenous (IV) or oral) and/or antibiotics. Severe exacerbations are recorded by the investigator and are defined as AECOPD that require hospitalization, observation for greater than 24 hours in an emergency department/urgent care facility, or results in death. For both moderate and severe events to be counted as two separate events, they are separated by at least 14 days between any course of systemic steroids/antibiotics or 14 days between discharge and new admission in case of hospitalization (severe events only).

For efficacy endpoint analysis, the primary population is the intent-to-treat (ITT) population of the former smokers cohort.

Separate randomizations are performed for the former smoker cohort, which is the primary population, and the current smoker cohort, which is a secondary population.

For the cohort of former smokers (primary population), participant randomization (to itepekimab 300 mg Q2W, itepekimab 300 mg Q4W, or matching placebo) is stratified using Interactive Voice Response System (IVRS)/Interactive Web Response System (IWRS) by country (some countries might be pooled together), screening blood eosinophil counts (<300/mm3 or ≥300/mm³), and controller therapy (double or triple) at baseline. The number of participants enrolled into each stratification group is controlled and monitored as follows:
Double controller therapy (LAMA+LABA or ICS+LABA): up to approximately 35% of participants.
Eosinophils ≥300 cells/mm³, approximately 35% of participants.

For the current smokers cohort (secondary population), participant randomization (to itepekimab 300 mg Q2W or matching placebo) is stratified (IVRS/IWRS) in the same way by country (some countries might be pooled together), screening blood eosinophil counts (<300 cells/mm³ or ≥300 cells/mm³), and controller therapy (double or triple) at baseline. The number of participants enrolled into each stratification group is controlled and monitored as follows:
Double controller therapy (LAMA+LABA or ICS+LABA): up to approximately 35% of participants.
Eosinophils ≥300 cells/mm³ approximately 35% of participants.

The study duration is outlined below:
Screening period (3-5 weeks).
Randomized investigational medicinal product (IMP) treatment period (52 weeks).
Post-IMP treatment follow-up period (20 weeks).

Participants have been on a SoC controller therapy for COPD for at least 3 months prior to screening (visit 1A) with a stable dose for controller therapy for ≥1 month prior to screening (visit 1A) and during the screening period. Participants stay on their established controller medication for COPD throughout the duration of the study, with the exception of systemic steroids and antibiotics used for AECOPD.

Former smoker participants who satisfy the eligibility criteria are randomized (1:1:1) to one of the following IMP treatment groups to be administered for 52 weeks:
Itepekimab 300 mg, administered as a single subcutaneous (SC) injection Q2W.
Itepekimab 300 mg, administered as a single SC injection Q4W with alternating SC injection of matching placebo at the 2-week interval between active IMP.
Placebo matching to itepekimab, administered as a single SC injection Q2W.

Current smoker participants who satisfy the eligibility criteria will be randomized (1:1) to one of the following IMP treatment groups to be administered for 52 weeks:
Itepekimab 300 mg, administered as a single SC injection Q2W.
Placebo matching to itepekimab, administered as a single SC injection Q2W.

### Number of Participants:

An approximate total of 1170 participants who are either former smokers (n=930) or current smokers (n=240) are enrolled and randomized separately into different cohorts in this Phase 3 study. Approximately 930 former smoker participants are randomized 1:1:1 into the 3 treatment arms. Approximately 310 participants are randomized per arm to receive either itepekimab 300 mg Q2W, itepekimab 300 mg Q4W, or matching placebo to itepekimab. Approximately 240 current smoker participants are randomized, with 120 participants each arm to receive either itepekimab 300 mg Q2W or matching placebo to itepekimab.

### Intervention Arms and Duration:

For former smokers, there are 3 treatment arms:
Arm A: Itepekimab 300 mg SC Q2W.
Arm B: Itepekimab 300 mg SC Q4W.
Arm C: Matching placebo SC Q2W.

Participants will be treated for 52 weeks.

For current smokers, there are 2 treatment arms:
Arm A: Itepekimab 300 mg SC Q2W
Arm B: Matching placebo SC Q2W Participants will be treated for 52 weeks.

### Type of Participant and Disease Characteristics:

Participants have a physician diagnosis of COPD for at least 1 year (based on the GOLD definition). Participants have a smoking history of ≥10 pack-years.

For former smokers: participants report that they are not currently smoking and smoking cessation occurred ≥6 months prior to screening (visit 1A) with an intention to quit permanently. Urine cotinine levels are tested at screening (visit 1A) and each subsequent visit during the study.

For current smokers: participants report that they are currently smoking tobacco (participant smoked at least one cigarette per day on average during the past seven days) at screening (visit 1A) and are not currently participating in or planning to initiate a smoking cessation intervention at screening (visit 1A) or during screening period.

Participants have moderate to severe COPD with post-BD FEV1/FVC ratio ≤0.70 and a post-BD FEV1% predicted ≥30% and <80% at screening (visit 1A) and at baseline/randomization (visit 2).

Participants have a COPD Assessment Test (CAT) score ≥10 at screening (visit 1A) and baseline/randomization (visit 2).

Participant-reported history of signs and symptoms of chronic bronchitis (chronic productive cough for at least 3 months in the year prior to screening in a participant in whom other causes of chronic cough (e.g., inadequately treated gastroesophageal reflux or chronic rhinosinusitis; or clinical diagnosis of bronchiectasis) is excluded).

Participants with a documented history of high exacerbation risk defined as having had ≥2 moderate or ≥1 severe exacerbations within the year prior to screening (visit 1A), with at least 1 exacerbation treated with systemic corticosteroids. At least 1 exacerbation occurred while participants were on their current controller therapy: moderate exacerbations are recorded by the investigator and are defined as acute worsening of respiratory symptoms that requires either systemic corticosteroids (IM, IV, or oral) and/or antibiotics (however, use of antibiotics alone does not qualify as a moderate exacerbation unless documentation is available that use of antibiotics was necessary for treatment of worsening symptoms of COPD); severe exacerbations are recorded by the investigator and are defined as AECOPD that require hospitalization or observation for >24 hours in emergency department/urgent care facility.

Participants with SoC controller therapy, for >3 months prior to screening (visit 1A) and at a stable dose of controller therapy for at least 1 month prior to the screening AND during the screening period, including either: double therapy (i.e., LAMA+LABA or ICS+LABA) or triple therapy (i.e., LAMA+LABA+ICS).

### Study Intervention(s):

### Investigational Medicinal Product(s)

Sterile itepekimab or matching placebo will be provided in prefilled syringes for SC administration. Each prefilled syringe contains a deliverable volume of 2 mL with an itepekimab concentration of 150 mg/mL (active) or 0 mg/mL (placebo).
Formulation: 2 mL solutions for injection (150 mg/mL)
Route(s) of administration: SC
Dose regimen: All participants will receive Q2W dosing to maintain the blind.

Former smoker participants on Q4W dosing regimen receive alternating doses of active IMP and placebo Q2W.

### Non-Investigational Medicinal Products(s)

Participants continue on their established controller therapy.
Formulation: dry powder inhaler (DPI), metered dose inhaler (MDI), or nebulizer.
Route(s) of administration: oral inhalation for LAMA, LABA, ICS, LAMA+LABA, ICS+LABA, or LAMA+LABA+ICS.
Dose regimen: as per prescribed.

### Reliever Medication (Albuterol/Salbutamol, Levalbuterol/Levosalbutamol, Ipratropium, Ipratropium/Albuterol)

Participants may administer albuterol/salbutamol, levalbuterol/levosalbutamol, ipratropium, or ipratropium/albuterol as reliever medication as needed during the study.
Formulation: DPI, MDI, nebulizer.
Route(s) of administration: oral inhalation, nebulization.
Dose regimen: as needed, per prescribed.

### Statistical Considerations:

### Primary Endpoints:

The primary analysis of the annualized rate of moderate-or-severe AECOPD during the 52-week placebo-controlled treatment period in former smokers will be performed following the ITT principle. The primary estimand is a treatment policy estimand. All moderate-or-severe AECOPD events during the 52-week treatment period are included and the observation duration will be from randomization to visit 28 (week 52). Participants who permanently discontinue IMP are asked and encouraged to return to the clinic for all remaining study visits, all off-treatment moderate-or-severe AECOPD during the planned 52-week treatment period will be included in the primary analysis. Similarly, if a participant withdraws from the study prior to the end of the 52-week treatment period, all observed moderate-or-severe AECOPD events up to the last contact date are included in the analysis, and in this case the observation duration will be from randomization to the last contact date. No imputation will be performed for the unobserved events that may happen after study discontinuation and up to week 52. The annualized rate of moderate-or-severe AECOPD is analyzed using a negative binomial regression model. The model includes the total number of moderate-or-severe AECOPD events that occur during the treatment period (up to week 52) as the response variable, with treatment group (placebo, itepekimab 300 mg SC Q2W, itepekimab 300 mg SC Q4W), region (pooled country), screening eosinophil strata (<300 cells/mm³, ≥300 cells/mm³), controller therapy (double, triple) strata, baseline disease severity (as % predicted post-bronchodilator (BD) FEV1 used as continuous variable), and total number of severe AECOPD events within 1 year prior to the study (0 or ≥1) as covariates. Log-transformed observation duration will be the offset variable. The treatment comparisons with placebo will be performed using a step-down procedure to compare itepekimab 300 mg SC Q2W versus placebo first; only if it is statistically significant, will the comparison of itepekimab 300 mg SC Q4W versus placebo be performed.

This estimand compares the rate of moderate-or-severe AECOPD for the participants randomly assigned to an itepekimab regimen versus placebo, regardless of what treatment the participants actually receive or whether the treatment regimens have been adhered to. It assesses the benefits of the treatment policy or strategy relative to placebo. The estimated annualized event rate for each treatment group and its two-sided 95% confidence intervals (CIs) are derived from the negative binomial model. The event rate ratio (RR) of each itepekimab regimen versus placebo, and the corresponding two-sided 95% CI and p-value are also be provided.

An on-treatment analysis to assess the efficacy of itepekimab excluding data measured when participants do not adhere to the treatment regimen as per protocol is performed and used to estimate the benefit when adhering to itepekimab treatment. In this analysis, only the AECOPD events observed during the on-treatment period (from first administration of IMP to last administration of IMP+14 days) are included. Off-treatment events of participants who prematurely discontinue treatment will be excluded from the analysis. A negative binomial model with the same set of covariates as specified in the primary analysis is used. This model includes moderate-or-severe AECOPD occurring during the on-treatment period as the response variable and the log-transformed duration of the treatment period will be the offset variable. This approach defines the estimand to evaluate the efficacy of itepekimab while on treatment.

### Secondary Endpoints:

Change from Baseline in Pre-BD FEV1 at Week 52

The main analysis of change from baseline in pre-BD FEV1 at week 52 is to assess the efficacy of itepekimab on pulmonary function in former smokers. The change from baseline in pre-BD FEV1 at week 52 is analyzed using a mixed-effect model with repeated measures (MMRM) approach. The model includes change from baseline in pre-BD FEV1 values up through week 52 as response variables, and treatment, age (continuous variable (years)), sex, baseline height (continuous variable), region (pooled country), screening eosinophil strata, controller therapy strata (double or triple), visit, treatment by-visit interaction, and baseline pre-BD FEV1 value (continuous variable) and baseline pre-BD FEV1-by-visit interaction as covariates. Participants who discontinue IMP before week 52 will be asked and encouraged to return to the clinic for all remaining study visits and the additional off treatment pre-BD FEV1 values measured up through week 52 will be included in the analysis. For participants who withdraw from the study before week 52, pre-BD FEV1 values will be missing after study discontinuation or last contact. No imputation is performed for missing values in this analysis. This estimand compares the change from baseline in pre-BD FEV1 for the participants randomly assigned to an itepekimab regimen versus the participants randomly assigned to the placebo arm, regardless of the treatment participants actually received. It assesses the benefits of the treatment policy or strategy relative to placebo.

An unstructured correlation matrix is used to model the within-participant errors. Parameters are estimated using restricted maximum likelihood method with the Newton-Raphson algorithm. Statistical inferences on-treatment comparisons for the change from baseline in pre-BD FEV1 at week 52 are derived from the mixed-effect model. Difference in least squares (LS) mean change from baseline, the corresponding 95% CI and p-value are provided for comparison of each itepekimab regimen against placebo.

To assess the treatment effect when the participants adhere to the study treatment as directed, on-treatment pre-BD FEV1 measurements are analyzed using a similar MMRM model as for the primary pre-BD FEV1 analysis, including the same set of covariates and estimation algorithm. The model includes on-treatment change from baseline in pre-BD FEV1 values up through week 52 as response variables. A pre-BD FEV1 value is considered as on-treatment if it is measured on or before the last dose date+14-days.

### Pulmonary Function—Former Smokers

The change from baseline in pre-BD FEV1 is determined at week 52. Change from baseline in post-BD FEV1 is determined at week 52. The change from baseline in pre-BD FEV1 is determined at week 24.

### AECOPD—Former Smokers

The time to first moderate-or-severe AECOPD is determined over the 52-week placebo-controlled treatment period.

### Severe AECOPD—Former Smokers

The annualized rate of severe AECOPD is determined over the 52-week placebo-controlled treatment period. The time to first severe AECOPD is determined over the 52-week placebo-controlled treatment period.

### Corticosteroid-Treated AECOPD—Former Smokers

The annualized rate of corticosteroid-treated AECOPD is determined over the 52-week placebo-controlled treatment period.

### Respiratory Symptoms—Former Smokers

The change from baseline in E-RS:COPD total score is determined at week 52.

### FEV1 Slope—Former Smokers

The rate of change in post-BD FEV1 (L) from baseline (post-BD FEV1 slope) is determined after 4-12 weeks.

### HRQoL as Assessed by SGRQ—Former Smokers

The change from baseline in SGRQ total score is determined at week 52. Proportion of participants with a decrease from baseline of at least four points in SGRQ total score is determined at week 52.

### Safety and Tolerability—Former Smokers

The incidence of TEAEs, AESIs, SAEs, and AEs leading to permanent treatment discontinuation are determined. Incidence of potentially clinically significant laboratory tests, vital signs, and ECG abnormalities are determined in the treatment-emergent period.

### PK Profile—Former Smokers

Functional itepekimab concentrations in serum are determined from baseline to end of study.

### Immunogenicity—Former Smokers

The incidence of treatment-emergent anti-itepekimab antibody responses are determined throughout the study.

### AECOPD—Current Smokers

The annualized rate of moderate-or-severe acute exacerbations of COPD (AECOPD) is determined over the 52-week placebo-controlled treatment period.

### Pulmonary Function—Current Smokers

The change from baseline in pre-BD FEV1 is determined at week 52.

### Safety and Tolerability—Current Smokers

The incidence of TEAEs, AESIs, SAEs, and AEs leading to permanent treatment discontinuation is determined. The incidence of potentially clinically significant laboratory, vital sign, and ECG abnormalities in the treatment-emergent period are determined.

### PK Profile—Current Smokers

Functional itepekimab concentrations in serum are determined from baseline to the end of the study.

### Immunogenicity—Current Smokers

The incidence of treatment-emergent anti-itepekimab antibody responses are determined throughout the study.

### Tertiary/Exploratory Endpoints:

### Healthcare Utilization—Former Smokers

The annualized number of days of healthcare resource utilization is determined over the 52-week placebo-controlled treatment period.

### Predictors of Mortality—Former Smokers

The annualized number of ER and hospital days related to AECOPD is determined. The proportion of participants with a BODE Index score >1 point decrease (=improvement) is determined at week 52.

### Pulmonary Function—Former Smokers

The proportion of participants with a pre-BD FEV1 improvement of ≥100 mL is determined at week 52.

### Reduction in Oral Corticosteroid and Antibiotic Use—Former Smokers

The number of days on oral corticosteroids and antibiotics is determined over 52 weeks.

### Respiratory Vital Signs—Former Smokers

The change from baseline in resting oxygen saturation is determined at week 52.

### Biomarkers—Former Smokers

The change from baseline in blood eosinophil levels and neutrophil levels are determined at weeks 4, 8, 12, 24, 36 and 52. The change from baseline is determined at weeks 4, 12, 24 and 52 for total blood IL-33 levels and for blood CRP levels.

### Gene Expression and Genetic Factors—Former Smokers and Current Smokers

Pharmacogenomics analyses, DNA sampling and RNA sampling may be performed.

1. A method for treating chronic obstructive pulmonary disease (COPD) in a subject in need thereof comprising administering to the subject:
an antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16.

2. The method of claim 1, wherein:
(a) one or more COPD-associated parameter(s) are improved in the subject;
(b) a score is improved in the subject on one or more questionnaires or assessments selected from the group consisting of COPD Assessment Test (CAT), St. George's Respiratory Questionnaire (SGRQ), Exacerbations of Chronic Obstructive Pulmonary Disease Tool (EXACT), Evaluating Respiratory Symptoms in COPD (E-RS), Body mass index, airflow Obstruction, Dyspnea, Exercise performance (BODE) index, and Euro Quality of Life-5 Dimension Questionnaire (EQ-5D);
(c) the COPD is moderate-to-severe COPD that is not well-controlled on a background therapy;
(d) the antibody or antigen-binding fragment thereof comprises a heavy chain variable region (HCVR) comprising the amino acid sequence of SEQ ID NO: 2 and a light chain variable region (LCVR) comprising the amino acid sequence of SEQ ID NO: 10, optionally wherein the antibody comprises a heavy chain comprising the amino acid sequence of SEQ ID NO: 18 and a light chain comprising the amino acid sequence of SEQ ID NO: 20; or
(e) the level of blood eosinophils is reduced.

3. The method of claim 2, wherein the one or more COPD-associated parameter(s) are selected from the group consisting of annualized rate of moderate-to-severe acute exacerbations of COPD (AECOPD), annualized rate of severe acute exacerbations of COPD (AECOPD), forced expiratory volume in 1 second (FEV1), peak expiratory flow (PEF), forced vital capacity (FVC), forced expiratory flow (FEF) 25%-75%, fractional exhaled nitric oxide (FeNO), frequency or dosage of a chronic obstructive pulmonary disease (COPD) reliever medication, frequency or dosage of a systemic corticosteroid, frequency or dosage of an antibiotic, daily steps, frequency or dosage of an oral corticosteroid, resting oxygen saturation, and resting respiratory rate.

4. The method of claim 3, wherein pre-bronchodilator FEV1 is improved in the subject or wherein annualized rate of AECOPD is reduced in the subject.

5-7. (canceled)

8. The method of claim 2, wherein the background therapy comprises therapy with at least two of the following: a long-acting β2 adrenergic agonist (LABA), a long-acting muscarinic antagonist (LAMA), and an inhaled corticosteroid (ICS),
optionally wherein the background therapy comprises: a LABA and a LAMA; a LABA and an ICS; a LAMA and an ICS: or a LABA, a LAMA and an ICS.

9-14. (canceled)

15. The method of claim 1, wherein the subject has a blood eosinophil count of greater than or equal to about 300 cells per μl, greater than or equal to about 250 cells per μl, less than 300 cells per μL, or less than about 250 cells per μL prior to treatment.

16-18. (canceled)

19. The method of claim 15, wherein pre-bronchodilator FEV1 and/or post-bronchodilator FEV1 is improved, or wherein post-bronchodilator FVC is improved.

20. (canceled)

21. The method of claim 1, wherein the subject is a current smoker, a former smoker or a non-smoker.

22. The method of claim 21, wherein the subject is a former smoker, optionally wherein the former smoker has a history of smoking greater than or equal to 10 packs per year, has quit smoking for at least 6 months, and/or intends to permanently quit smoking.

23. The method of claim 22, wherein
(a) annualized rate of moderate-to-severe AECOPD events is reduced in the subject;
(b) time to first moderate-to-severe AECOPD event is reduced;
(c) pre-bronchodilator FEV1 and/or post-bronchodilator FEV1 is improved;
(d) post-bronchodilator FVC is improved;
(e) rate of decline of FEV1 is decreased; or
(f) lung function is maintained or a lung function decline is reduced.

24-29. (canceled)

30. The method of claim 1, wherein the antibody or antigen binding fragment thereof is administered:
at a dose of about 0.1 mg to about 600 mg, about 100 mg to about 400 mg, or about 300 mg; or
every week (q1w), every other week (q2w), every three weeks (q3w), every four weeks (q4w), every five weeks (q5w), every 6 weeks (q6w), every seven weeks (q7w) or every eight weeks (q8w).

31-34. (canceled)

35. The method of claim 30, wherein pre-bronchodilator FEV1 is improved within 4 weeks of the first administration of the antibody or antigen-binding fragment thereof and/or FEV1 is maintained during treatment or wherein the antibody or antigen-binding fragment thereof is administered subcutaneously.

36. (canceled)

37. The method of claim 35, wherein the antibody or antigen-binding fragment thereof is administered as two injections, or wherein the antibody or antigen-binding fragment thereof is administered subcutaneously using an autoinjector, a needle and syringe, or a pen delivery device.

38. (canceled)

39. A method for treating chronic obstructive pulmonary disease (COPD) in a subject in need thereof comprising administering to the subject:
an initial dose of about 300 mg of an antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16; and
one or more subsequent doses of about 300 mg of the antibody or antigen-binding fragment thereof; or
a method for treating moderate-to-severe chronic obstructive pulmonary disease (COPD) in a subject in need thereof comprising administering to the subject:
an initial dose of about 300 mg of an antibody that specifically binds interleukin-33 (IL-33), wherein the antibody comprises a heavy chain comprising the amino acid sequence of SEQ ID NO: 18, and a light chain comprising the amino acid sequence of SEQ ID NO: 20; and
one or more subsequent doses of about 300 mg of the antibody,
wherein the antibody is administered subcutaneously every other week; or
a method for treating moderate-to-severe chronic obstructive pulmonary disease (COPD) in a subject in need thereof comprising administering to the subject:
an initial dose of about 300 mg of an antibody that specifically binds interleukin-33 (IL-33), wherein the antibody comprises a heavy chain comprising the amino acid sequence of SEQ ID NO: 18, and a light chain comprising the amino acid sequence of SEQ ID NO: 20; and
one or more subsequent doses of about 300 mg of the antibody,
wherein the antibody is administered subcutaneously every four weeks.

40. The method of claim 39, wherein the antibody or antigen-binding fragment thereof comprises a heavy chain variable region (HCVR) comprising the amino acid sequence of SEQ ID NO: 2 and a light chain variable region (LCVR) comprising the amino acid sequence of SEQ ID NO: 10.

41. (canceled)

42. (canceled)

43. The method of claim 39, wherein one or more COPD-associated parameter(s) are improved in the subject, optionally wherein the one or more COPD-associated parameter(s) are selected from the group consisting of annualized rate of moderate-to-severe acute exacerbations of COPD (AECOPD), forced expiratory volume in 1 second (FEV1), rate of decline in FEV1, peak expiratory flow (PEF), forced vital capacity (FVC), forced expiratory flow (FEF) 25%-75%, fractional exhaled nitric oxide (FeNO), frequency or dosage of a COPD reliever medication, frequency or dosage of a systemic corticosteroid, and frequency or dosage of an antibiotic.

44. (canceled)

45. The method of claim 43, wherein pre-bronchodilator FEV1 is improved, or wherein the annualized rate of moderate-to-severe acute exacerbations of COPD (AECOPD) is reduced in the subject.

46. (canceled)

47. The method of claim 1, wherein at least two additional therapeutic agents are administered to the subject,
optionally wherein the at least two additional therapeutic agents are selected from the group consisting of a long-acting β2 adrenergic agonist (LABA), a long-acting muscarinic antagonist (LAMA), and an inhaled corticosteroid (ICS), or
optionally wherein a LABA and a LAMA a LABA and an ICS; a LAMA and an ICS; or a LABA, a LAMA and an ICS are administered.

48-51. (canceled)

52. A method for reducing annualized rate of moderate-to-severe acute exacerbations of chronic obstructive pulmonary disease (AECOPD) in a subject having moderate-to-severe chronic obstructive pulmonary disease (COPD) comprising administering to the subject:
an initial dose of about 300 mg of an antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16; and
one or more subsequent doses of about 300 mg of the antibody or antigen-binding fragment thereof; or
a method for reducing annualized rate of moderate-to-severe acute exacerbations of chronic obstructive pulmonary disease (AECOPD) in a subject having moderate-to-severe chronic obstructive pulmonary disease (COPD) comprising administering to the subject:
an initial dose of about 300 mg of an antibody that specifically binds interleukin-33 (IL-33), wherein the antibody comprises a heavy chain comprising the amino acid sequence of SEQ ID NO: 18, and a light chain comprising the amino acid sequence of SEQ ID NO: 20; and
one or more subsequent doses of about 300 mg of the antibody,
wherein the antibody is administered subcutaneously every other week; or
a method for reducing annualized rate of moderate-to-severe acute exacerbations of chronic obstructive pulmonary disease (AECOPD) in a subject having moderate-to-severe chronic obstructive pulmonary disease (COPD) comprising administering to the subject:
an initial dose of about 300 mg of an antibody that specifically binds interleukin-33 (IL-33), wherein the antibody comprises a heavy chain comprising the amino acid sequence of SEQ ID NO: 18, and a light chain comprising the amino acid sequence of SEQ ID NO: 20; and
one or more subsequent doses of about 300 mg of the antibody,
wherein the antibody is administered subcutaneously every other week, wherein the subject is a former smoker; or
a method for reducing annualized rate of moderate-to-severe acute exacerbations of chronic obstructive pulmonary disease (AECOPD) in a subject having moderate-to-severe chronic obstructive pulmonary disease (COPD) comprising administering to the subject:
an initial dose of about 300 mg of an antibody that specifically binds interleukin-33 (IL-33), wherein the antibody comprises a heavy chain comprising the amino acid sequence of SEQ ID NO: 18, and a light chain comprising the amino acid sequence of SEQ ID NO: 20; and
one or more subsequent doses of about 300 mg of the antibody,
wherein the antibody is administered subcutaneously every four weeks; or
a method for reducing annualized rate of moderate-to-severe acute exacerbations of chronic obstructive pulmonary disease (AECOPD) in a subject having moderate-to-severe chronic obstructive pulmonary disease (COPD) comprising administering to the subject:
an initial dose of about 300 mg of an antibody that specifically binds interleukin-33 (IL-33), wherein the antibody comprises a heavy chain comprising the amino acid sequence of SEQ ID NO: 18, and a light chain comprising the amino acid sequence of SEQ ID NO: 20; and
one or more subsequent doses of about 300 mg of the antibody,
wherein the antibody is administered subcutaneously every four weeks, wherein the subject is a former smoker.

53. The method of claim 52, wherein the antibody or antigen-binding fragment thereof comprises a heavy chain variable region (HCVR) comprising the amino acid sequence of SEQ ID NO: 2 and a light chain variable region (LCVR) comprising the amino acid sequence of SEQ ID NO: 10.

54-57. (canceled)
